(19) United States
(12) Patent Application Publication
(10) Pub. No.: US 2025/0387466 A1
(43) Pub. Date: Dec. 25, 2025

(54) COMPOSITIONS OF NUCLEIC ACID NANOSTRUCTURES FOR VACCINES AND METHODS OF USE THEREOF

(71) Applicant: Massachusetts Institute of Technology, Cambridge, MA (US)

(72) Inventors: Mark Bathe, Cambridge, MA (US); Remi Veneziano, Manassas, VA (US); Eike-Christian Wamhoff, Brookline, MA (US); Tyson Moyer, Boston, MA (US); Benjamin Joseph Read, Cambridge, MA (US); Anna Romanov, Cambridge, MA (US); Grant Alexander Knappe, Boston, MA (US); Darrell Irvine, Arlington, MA (US)

(21) Appl. No.: 19/257,179

(22) Filed: Jul. 1, 2025

Related U.S. Application Data
(63) Continuation-in-part of application No. 17/819,204, filed on Aug. 11, 2022.
Application No. 17/819,204 is a continuation-in-part of application No. 16/752,394, filed on Jan. 24, 2020, now Pat. No. 11,419,932.
(60) Provisional application No. 62/796,472, filed on Jan. 24, 2019.
(60) Provisional application No. 63/333,498, filed on Apr. 21, 2022.
(60) Provisional application No. 63/666,555, filed on Jul. 1, 2024.

(51) Int. Cl. A61K 39/21; A61K 39/00; A61P 37/04; C12N 7/00

(52) U.S. Cl. CPC A61K 39/21; A61P 37/04; C12N 7/00; A61K 2039/5258; A61K 2039/55516; A61K 2039/6025; A61K 2039/627; C12N 2740/16022; C12N 2740/16023; C12N 2740/16034; C12N 2740/16071

(57) ABSTRACT

Compositions containing a nucleic acid nanostructure having a desired geometric shape and an antigen and/or immunostimulatory agent(s) bound to its surface are provided. The nanostructure design allows for control of the relative position and/or stoichiometry of the immunostimulatory agent(s) bound to its surface. The antigen and/or immunostimulatory agent(s) displayed on the nanostructure surface are arranged with the preferred number, spacing, and 3D organization to elicit a robust immune response. The displayed antigen can be eOD-GT8. The immunostimulatory agent can be, e.g., T cell epitope such as a pan HLA DR-binding epitope (PADRE) and/or a lectin such as MBL or C3, or ligand thereof such as a glycan including mannose. Also provided are antigen-T cell epitope fusions such as eOD-PADRE and nanostructures presenting the same. The immunostimulatory compositions may thus be useful as immunogens, vaccines, adjuvants, and the like. Methods of inducing immune responses are also provided.

### CROSS-REFERENCE TO RELATED APPLICATIONS

This application claims the benefit of and priority to U.S. Provisional Application No. 63/666,555, filed Jul. 1, 2024, and is a continuation-in-part (CIP) of U.S. application Ser. No. 17/819,204, filed Aug. 11, 2022, which claims the benefit of and priority to U.S. Provisional Application No. 63/333,498, filed Apr. 21, 2022, and which is a continuation-in-part (CIP) of U.S. application Ser. No. 16/752,394, filed Jan. 24, 2020, now U.S. Pat. No. 11,419,932, which claims the benefit of U.S. Provisional Application No. 62/796,472, filed Jan. 24, 2019. U.S. application Ser. No. 17/819,204, U.S. application Ser. No. 16/752,394, U.S. Provisional Application No. 63/666,555, U.S. Provisional Application No. 63/333,498, and U.S. Provisional Application No. 62/796,472, are hereby incorporated by reference in their entireties.

### STATEMENT REGARDING FEDERALLY SPONSORED RESEARCH

This invention was made with government support under MH112694, AI162307, and EB026008 awarded by the National Institutes of Health, and N00014-16-1-2181 awarded by the Office of Naval Research. The government has certain rights in the invention.

### REFERENCE TO SEQUENCE LISTING

The Sequence Listing submitted as an xml file named “MIT_20929_H_CIP_2.xml”, created on Jun. 30, 2025, and having a size of 20,731 bytes is hereby incorporated by reference pursuant to 37 C.F.R. § 1.834(c)(1).

### FIELD OF THE INVENTION

The present invention generally relates to immunostimulatory compositions, and in particular, the use of nucleic acid nanostructures having a specified geometric shape, for example a shape that mimics a natural macromolecular assembly, as a platform for the user-defined, programmable display of immunostimulatory molecules.

### BACKGROUND OF THE INVENTION

Formulating a protective vaccine against HIV-1 has proven difficult due to high mutability of the virus and inefficient neutralization of antibodies generated by natural infection or vaccination with native HIV Envelope trimers¹⁻³. Broadly neutralizing antibodies (bnAbs), which target conserved and essential epitopes such as the CD4 binding site, have been discovered in a subset of infected individuals⁴, and eliciting bnAbs has emerged as a key goal of vaccine design for HIV and other difficult-to-neutralize pathogens⁵. Achieving bnAbs through vaccination necessitates B cells to undergo great extents of somatic hypermutation and affinity maturation, a Darwinian-like selection process which occurs in microanatomical centers in lymph nodes called germinal centers (GCs), where B cells mutate their B cell receptors (BCRs), proliferate, and compete for access to antigen and follicular T cell help^{6,7}. Promising immunization approaches to promote longer germinal centers for bnAb maturation are rational design of germline-targeting immunogens to target bnAb precursors^{8,9}, glycan engineering of Env immunogens to change immunodominance patterns¹⁰, and multivalent nanoparticle display of antigens to lower the affinity threshold for B cell activation^{9,11,12}. Increasing antigen valency is a potential strategy to elevate serum responses; thus, it is desirable for nanoparticulate vaccine designs which associate higher valency with stronger humoral immunity, by lowering activation threshold for B cells and amplifying downstream signaling. Attempts have been made to use protein nanoparticles to interrogate the effects of valency and antigen spacing in vivo, but due to constraints in protein design, it has been difficult to assess the role of antigen organization without further altering nanoparticle symmetry, diameter, antigen spacing, or scaffold-intrinsic T cell help.

Peptide- and protein-based vaccines form the major class of vaccination strategies globally. Inactivated or otherwise attenuated antigenic particles are typically used in vaccine formulations to display protein or peptide antigens to stimulate and train the immune response. Adjuvants including small molecules and nucleic acids are often co-formulated in these nanoparticles to further stimulate immune activation.

The field of nucleic acid nanotechnology has over the past several decades developed technologies enabling the fabrication of programmable DNA-based assemblies of prescribed size, geometry, rigidity, and chemical composition (Jun, et al., Nucleic Acids Research, 49 (18), 10265-10274 (2021), Pettersen, et al., Journal of Computational Chemistry, 25 (13), 1605-1612 (2004), Benson, et al., Angewandte Chemie International Edition, 55 (31), 8869-8872 (2016), Jun, et al., ACS Nano (2019), Benson, et al., Nature, 523 (7561), 441-444 (2016), Dietz, et al., Science, 325 (5941), 725-730 (2009), Castro, et al., Nature Methods, 8 (3), 221-229 (2011), Douglas, et al., Nucleic Acids Research, 37 (15), 5001-5006 (2009), Veneziano, et al., Science, 352 (6293), 1534-1534 (2016)). These nanomaterials now represent a toolbox for the design and fabrication of nanodevices capable of interacting with diverse cellular environments (Veneziano, et al., Nature Nanotechnology, 15 (8), 716-723 (2020), Lee, et al., Nature Nanotechnology, 7 (6), 389-393 (2012)). One approach to designing nucleic acid nanostructures on the 10-100 nm scale is the concept of DNA origami, wherein programmed regions of a long, single-stranded DNA scaffold that are far apart in sequence space are brought into spatial proximity through the hybridization of small, single-stranded DNA staples. Scalable scaffold production strategies using M13 bacteriophage engineering and bioproduction have also now provide control over scaffold sequence composition and length, expanding the accessible design space for such wireframe nucleic acid nanoparticles (NANPs).

The ability to chemically functionalize NANPs also permits the attachment of therapeutic nucleic acid (TNA) cargo such as siRNA and miRNA, as well as small molecules, aptamers, peptides, and proteins with nanometer level spatial control for active targeting or immune cell stimulation (Veneziano, et al., Nature Nanotechnology, 15 (8), 716-723 (2020), Douglas, et al., Science, 335 (6070), 831-834 (2012), Knappe, et al., ACS Nano, 15 (9), 14316-14322 (2021)) and enhances the potential of NANPs to interface with biological systems in vitro and in vivo (Liu, et al., Nano Letters, 12 (8), 4254-4259 (2012), Irvine, et al., Chemical Reviews, 115 (19), 11109-11146 (2015)). However, the ability to selectively and controllably modulate specific immunostimulatory pathways in vivo using DNA based NANPs has only been examined to a limited extent (Hong, et al., Molecules, 24 (6), 1094 (2019), Surana, et al., Nature Nanotechnology, 10 (9), 741-747 (2015), Hong, et al., Nano Letters, 18 (7), 4309-4321 (2018), SchUller, et al., ACS Nano, 5 (12), 9696-9702 (2011)).

It is an object of the invention to provide alternative and/or improved immunogenic compositions containing immuno-modulatory agents and vaccine formulations formed therewith, and methods of use thereof.

It is also an object of the invention to provide a platform generally applicable to the controlled organization and preferred display of any immuno-modulatory agents for eliciting or otherwise manipulating an immune response.

It is another object of the invention to provide nanoscale structures that enable the user-defined display and organization of immuno-modulatory agents to provide tunable control of the intensity of immune-pathway activation and methods of use thereof.

It is a further object of the invention to provide methods of displaying TLR agonists in a preferred form for use in stimulation of TLR responses in a selective and user-defined manner in vivo.

It is a further object of the invention to provide methods of inducing an immune response or protective immunity or immune tolerance in a subject, and methods of treating subjects having, or at risk of having, a disease or condition.

### SUMMARY OF THE INVENTION

It has been established that nucleic acid nanostructures including one or more antigens and/or immunostimulatory agents can be designed to illicit user-defined modulation of immune responses in a subject. Elements including the structure of an antigen and/or immunostimulatory agent, the number of copies of the antigen and/or immunostimulatory agent, the spacing of copies of the antigen and/or immunostimulatory agent (i.e., distance between two copies of antigen and/or immunostimulatory agent), the location of the antigen and/or immunostimulatory agent on a nanostructure, the rigidity/flexibility of the antigen and/or immunostimulatory agent; the dimensionality of the antigen and/or immunostimulatory agent, the topology of the nanostructure, the ultra-structural organization of the nanostructure, and the geometric shape of the nanostructure can affect the magnitude of the immune response that can be induced by the antigen and/or immunostimulatory agent when presented to immune cells by the nanostructure.

Nanostructures can be designed and created that vary one or more of these elements in systematic or non-systematic ways, and the most effective immunogen can be selected. Thus, antigen-bound nucleic acid nanostructures with improved immunogenicity are provided.

Immunodominance and B cell competition in germinal centers create a challenging environment for the selection and survival of rare, low affinity B cells clones, such as VRCO1-class precursors which target the CD4bs in HIV. In contrast to protein nanoparticles such as the eOD-60mer, where antigen-specific and scaffold-specific B cells compete for the same helper T cells (due to presentation of scaffold-derived peptides on both antigen- and scaffold-specific B cells), GCs formed by the disclosed nucleic acid nanostructure can reduce B cell competition due to the T-independent nature of the scaffold. Furthermore, incorporating synthetic T cell helper epitopes into the nanostructures and/or formulations thereof can provide T cell help only to antigen-specific B cells, which may boost the antigen-specific composition of germinal centers. Thus, this technology offers precise nanoscale antigen organization on inert scaffolds with focused T cell help for enhancing germinal center responses post-prime and minimizing unwanted competitor B cell responses.

Any of the nanostructures can further include one or more moieties incorporated in and/or linked to the nanostructure. Such moieties include, for example, adjuvants, targeting molecules, therapeutic agents, stabilizing agents, passivating agents, etc.

Cationic polymers and minor groove binders (as monomers, oligomers or polymers) may be used to coat the DNA nanoparticles for stabilization from endonuclease degradation.

In particular, minor groove binders may act as tethers for covalent modifications of nucleic acids, for example to develop cross-linking strategies for DNA nanoparticles. These approaches can be combined, e.g. using brush or block copolymers, with PEGylation to further improve stabilization and passivation.

Pharmaceutical compositions and immunogenic compositions including the nucleic acid nanostructure are also provided. The composition can include, for example, a pharmaceutically acceptable carrier, diluent, preservative, excipient, or combination thereof. The nucleic acid nanostructure can be present in an effective amount to induce an immune response in a subject in need thereof, with or without the aid of an adjuvant. In some embodiments, the immunostimulatory agent is, or includes an adjuvant. The composition can also be free from adjuvant. The adjuvant can form part of the nanostructure or can be independent therefrom or otherwise unlinked or unbound thereto. The adjuvant can be in an effective amount to enhance the immune response relative to administration of the nucleic acid nanostructure alone.

Methods of using nucleic acid nanostructures are also provided. The methods can include, for example, administering to a subject in need thereof an effective amount of nanostructure to induce or enhance an immune response, induce or enhance protective immunity against an infectious agent, disease, or condition, treat or prevent a disease or condition, induce or enhance the production of neutralizing antibodies or inhibitory antibodies, or a combination thereof in a subject in need thereof. In some forms, the nanostructures (1) induce or increase a humoral response compared to soluble monomer immunization, (2) induce or increase complement activation via the lectin-pathway for improved lymph node follicle trafficking, and/or (3) increase the frequency of antigen-specific germinal center B cells. The methods can further include administering the subject one or more additional agents, such as adjuvants, one or more therapeutic agents, or a combination thereof. The adjuvant and/or therapeutic agent can be in the same or a different composition from the immunostimulatory agent-bound nanostructure. The adjuvant and/or therapeutic agent can be administered at the same or a different time from the antigen. In some embodiments, the subject is a mammal such as a human.

In particular embodiments, the methods include prophylactic treatment of HIV in a subject in need thereof by administering the subject an effective amount of an antigen-bound nanostructure to induce an immune response against HIV in the subject.

Methods of vaccination are also provided. The methods typically include administering to a subject an effective amount of an immunostimulatory agent-bound nanostructure to induce an immune response in the subject. In more specific embodiments, the vaccination is against HIV and the immunostimulatory agent is an antigen such as eOD-GT8.

### BRIEF DESCRIPTION OF THE DRAWINGS

FIG. 1A is a schematic showing an eOD-GT8 60-mer nanoparticle (left) and an icosahedral DNA-NP (right) designed to assemble the eOD-GT8 antigen in a controlled manner. FIG. 1B is a schematic illustrating how an icosahedron and 6-helix bundle structures were used to explore the stoichiometry, inter-antigen distance, and dimensionality of eOD-GT8 antigen presentation. FIG. 1C is a Cadnano design illustration of a DNA 6 helix bundle (6HB) structure side-by-side with a corresponding model generated with CanDo. FIGS. 1D and 1E are illustrations of overhang placement on the edge of the DNA nanostructures: DNA icosahedron (FIG. 1D) DNA 6HB (FIG. 1E). FIG. 1F is a schematic of a PNA linker designed for antigen attachment to DNA nanostructures.

FIG. 2A is a schematic illustrating folding of the two types of DNA-NPs (6-helix bundle and DNA icosahedron) that were designed and used for 1D and 3D presentation of antigens. FIG. 2B is a schematic demonstrating an overview of the antigen conjugation protocol to attach eOD-GT8 antigens to the DNA nanostructures using PNA strands complementary to DNA overhangs on the DNA nanostructures. FIGS. 2C-2F are line graphs (FIGS. 2C and 2E) and bar graphs (FIGS. 2D and 2F) showing quantification of B cell (human Ramos cell) activation upon exposure to DNA NPs modified with eOD-GT8 at 5 nM (FIGS. 2C and 2D) and 0.5 nM (FIGS. 2E and 2F) eOD-GT8. Raw fluorescence of cells loaded with Fluo-4 calcium probe normalized by unstimulated levels, less buffer-only control curves (FIGS. 2C and 2E) and areas under the curves normalized to maximum in repeat (FIGS. 2D and 2F) are shown. FIG. 2G is a line graph showing the effect of valency of eOD-GT8 on DNA icosahedron antigen presentation.

FIG. 3A is a graph showing quantification of B cell activation (assayed through Calcium release) upon exposure to DNA NP eOD-GT8 dimers having inter-antigen distances between 7 nm and 80 nm. The bottom schematic illustrates possible organizations of VRCO1 antibodies on 6HB displaying eOD-GT8 dimers at 7 and 80 nm. FIG. 3B is a graph showing quantification of B cell activation (assayed through Calcium release) upon exposure to eOD-GT8 dimers attached either to flexible polymeric scaffolds (ssDNA or PEG) or rigid 6HB DNA NP eOD-GT8 dimer structures. Numbers above the bars indicate the full extended length of the polymeric scaffold or the inter-antigen distance on 6HB DNA NP eOD-GT8 dimers.

FIGS. 4A-4D are graphs showing comparisons of B cell activation (assayed through Calcium release) upon exposure to 6 helical bundle (6HB) or icosahedral (Ico) structures presenting the eOD-GT8 antigens at various inter-antigen distances including, 7 and 3 nm respectively (FIG. 4A), 11 and 11 nm respectively (FIG. 4B), 14 and 15 nm respectively (FIG. 4C), and 17 and 22 nm respectively (FIG. 4D). In FIGS. 4A-4D, raw fluorescence of cells loaded with Fluo-4 calcium probe normalized by division by unstimulated levels and then subtraction of buffer-only control curves are shown. FIGS. 4E-4F are graphs showing quantification of B cell activation upon exposure to 3D (FIG. 4E) and 1D (FIG. 4F) DNA NP structures presenting 5 eOD-GT8 antigens (5-mer). In FIGS. 4E-4F, data is normalized to the condition showing maximum activation.

FIG. 5A is a scatter plot showing that the total intensity of eOD-GT8 is highly correlated with the intensity of the B cell receptor (BCR), confirming specific binding of nanoparticles to the B cell receptor. FIG. 5B is a bar graph showing quantification of total pSyk intensity per cell upon exposure to the indicated DNA NP eOD structures. Ramos cells were labeled with an anti-phospho-Syk antibody after fixation and the total pSyk intensity per cell was determined. FIG. 5C is a bar graph showing quantification of the internalized fraction of eOD estimated by segmenting the cell surface using phalloidin staining. Total internal eOD fluorescence was divided by total cellular eOD fluorescence on a cell-by-cell basis. Error bars denote the standard error of the mean fluorescence between the cells.

FIG. 5D is a bar graph showing quantification of flow cytometry of labeled eOD-DNA nanostructures (eOD 30mer, 6 HB 7, and 6HB 28) binding to B cells. FIG. 5E is a bar graph showing cellular eOD intensity (in cell) for each construct (6HB7, 6HB28, ico30) at (from left-to-right for each construct) control, 1 minute, 5 minutes, and 30 minutes). FIG. 5F is a bar graph showing pixel-wise pearson correlation between B cell receptor and eOD intensity (correlation coefficient) for each construct (6HB7, 6HB28, ico30) at (from left-to-right for each construct) control, 1 minute, 5 minutes, and 30 minutes).

FIGS. 6A-6D are line graphs (FIGS. 6A and 6C) and bar graphs (FIGS. 6B and 6D) showing quantification of B cell (human Ramos cell) activation upon exposure to pentagonal bipyramid DNA NPs modified with eOD-GT8 at 5 nM (FIGS. 6A and 6B) and 1 nM (FIGS. 6C and 6D) eOD-GT8. FIG. 6E is a line graph and FIG. 6F is a bar graph showing quantification of B cell (human Ramos cell) activation upon exposure to icosahedral and pentagonal bipyramid DNA NPs modified with eOD-GT8 at 2 nM eOD-GT8. Raw fluorescence of cells loaded with Fluo-4 calcium probe normalized by unstimulated levels, buffer-only control curves (FIGS. 6A, 6C and 6E) and areas under the curves normalized to the maximum in repeat (FIGS. 6B, 6D and 6F) are shown. FIGS. 6G and 6H are bar graphs showing a comparison of inter-immunogen distances for icosahedral (FIG. 6G) and pentagonal bipyramidal (FIG. 6H) DNA nanostructures bearing 10 copies of eOD-GT8.

FIG. 7A is a line graph and FIG. 7B is a bar graph, each showing quantification of B cell (primary mouse 3-83 splenocyte) activation upon exposure to pentagonal bipyramid DNA NPs modified with 3-83 peptide antigens p5 or p31 at 1 nM peptide. Raw fluorescence of cells loaded with Fluo-4 calcium probe normalized by unstimulated levels, buffer-only control curves (FIG. 7A) and areas under the curves normalized to the maximum in repeat (FIG. 7B) are shown.

FIGS. 8A-8E illustrate design and fabrication of DNA-VLP panel with covalent conjugation of eOD-GT8. FIG. 8A shows atomic models of icosahedral DNA origami nanoparticles with diameters of 30 or 40 nm (d30_and d40_) and eOD-GT8 valency of 30 or 60. FIGS. 8B-8C are graphs showing predicted glycan densities (8B) and inter-antigen distances (8C), calculated as the average distance to neighboring 5 antigens based on atomic models. FIGS. 8D-8E are graphs showing MBL deposition and (8D) C3 deposition (8E) on VLPs following incubation in fresh mouse serum detected by ELISA, for each of eOD-GT8, icosahedral DNA origami nanoparticles with diameters of 30 nm (d30), or 40 nm (d40), and with conjugated eOD-GT8 valency of 30 (30mer) or 60 (60mer), respectively.

FIGS. 9A-9E illustrate how high density antigen display enhances lectin pathway activation, FDC targeting, and antigen-specific GC B cell responses. FIG. 9A shows Normalized calcium signaling (flux) over time of glVRC01 B cells incubated with each of 25 eOD formulations d30_30mer, d40_30mer, d40_60mer, d30_60mer and eOD-GT8;

FIGS. 9B-9C are graphs showing number of GC B cells (9B), and number of Tfh cells (9C); and FIGS. 9D-9E are graphs showing frequencies (9D), and counts (9E), of eOD++GC B cells, respectively.

FIGS. 10A-10I show Incorporating synthetic helper T cell epitopes boosts GC size after DNA-VLP vaccination. FIG. 10A shows Normalized calcium signaling (flux) over time of glVRC01 B cells incubated with each of eOD-GT8, eOD-PADRE, d30_60mer, d30_60mer -PADRE, and p60mer, respectively; FIG. 10B shows Anti-eOD-GT8 IgG titers determined by ELISA (against eOD-GT8 without PADRE), as AUC values (log 10);

FIG. 10C shows Anti-PADRE IgG titers determined by ELISA against biotinylated PADRE peptide, as AUC values (log 10); FIGS. 10D-10E show frequencies (% GC out of total B cells) (10D), and counts of germinal center B cells (B220+/CD381o GL7hi) (10E);

FIGS. 10F-10G show total frequencies (% Tfh out of CD4 T cells)(10F), and counts of Tfh cells (CD4+/CXCR5+PD1+)(10G); FIGS. 10H and 10I show frequencies (10H) and total counts (10I) of antigen-specific GC B cells frequencies (% eOD++out of GC B cells), and counts of antigen-specific eOD++GC B cells.

FIGS. 11A-11J shows DNA-VLPs produce focused germinal centers and prime VRCO1-class precursors in humanized mice. FIG. 11A is a schematic of humanized VH1-2 mice (n=8/group) were primed s.c. with 5 g d30_60mer-PADRE or p60mer with SMNP, and inguinal lymph nodes were harvested on day 14; the lymphocytes were split into four samples and each stained with GC markers and fluorescently labeled antigen probes (eOD-60mer, bare lumazine synthase, or bare DNA-VLP (d30) probes); FIG. 11B shows total counts of B220+/CD38loGL7hi GC B cells for each of d30_60mer-PADRE, and p60mer, respectively; FIG. 11C shows frequency of eOD-60mer++cells out of IgM- IgD- GC B cells (11C) for each of d30_60mer-PADRE, and p60mer, respectively; FIGS. 11D-11E show modal frequency of eOD-60mer++eOD-CD4bs-KO- (CD4bs+) out of IgM- IgD- GC B cells for each of d30_60mer-PADRE, and p60mer, respectively; FIG. 11F shows frequency of LumSyn++cells out of IgM- IgD- GC B cells for each of d30_60mer-PADRE, and p60mer, respectively; FIGS. 11G-11H show Fraction of germinal center B cells that bound to each set of antigen or scaffold probes represented as parts-of-whole plot, for each pf DNA scaffold (11G) or LumSyn scaffold (11H), respectively; the slice represents on-target CD4bs-specific GC B cells; FIG. 11I shows frequency of DNA-VLP++cells out of IgM-IgD- GC B cells; FIG. 11J shows CD4bs:competitor ratio.

FIGS. 12A-12F show design and efficacy of multimerized core-g28v2 antigens on DNA-VLP scaffolds. FIG. 12A depicts the d30_60mer-core-g28v2, showing the DNA scaffold and extent of glycosylated core-g28v2; FIG. 12B is a graph of binding of core-g28v2 and azido-modified core-g28v2 to murine VRCO1 antibody as measured by ELISA, showing Absorbancve (450-540 nm) over mVRC01 μg/ml for each of core gp28v2, core gp28v2-azide and blank samples; FIG. 12C is a photomicrograph image of the gel electrophoresis of bare d30 VLPs and core-functionalized DNA-VLPs; FIG. 12D is a graph of Dynamic light scattering of bare and core-functionalized DNA-VLPs, showing size distribution (Diameter/nm) over % Number; FIGS. 12E-12F are graphs showing data from complement deposition assays comparing MBL (12E) and C3 (12F) binding from mouse serum to monomeric eOD and core or multimerized into DNA-VLPs (d30_60mer-eOD and d30_60mer-core) immobilized on ELISA plates. Protein-based eOD-60mer nanoparticles are used as a positive control for the assays.

FIGS. 13A-13G show GC priming in MBL- and C1q-deficient mice. FIG. 13A is a schematic overview of vaccination study. WT, MBL KO, and C1q KO mice were vaccinated with eOD-60mer or DNA-VLPs and GCs were analyzed after 2 weeks; FIG. 13B shows quantification of MBL deposition on nanoparticles in WT or MBL KO sera by ELISA; FIG. 13C shows quantification of C1q deposition on nanoparticles in WT or C1q KO sera by ELISA; FIG. 13D shows Anti-eOD IgG on day 14, reported as AUC; FIG. 13E shows counts of GC B cells; FIG. 13F shows counts of antigen-specific GC B cells;

FIG. 13G shows counts of Tfh cells. Statistical significance was determined by one-way ANOVAs (calculated within each treatment group).

### DETAILED DESCRIPTION OF THE INVENTION

### I. Definitions

The terms “nucleic acid molecule,” “nucleic acid sequence,” “nucleic acid fragment,” “oligonucleotide” and “polynucleotide” are used interchangeably and are intended to include, but not limited to, a polymeric form of nucleotides that can have various lengths, either deoxyribonucleotides (DNA) or ribonucleotides (RNA), or analogs or modified nucleotides thereof, including, but not limited to locked nucleic acids (LNA) and peptide nucleic acids (PNA). An oligonucleotide is typically composed of a specific sequence of four nucleotide bases: adenine (A); cytosine (C); guanine (G); and thymine (T) (uracil (U) for thymine (T) when the polynucleotide is RNA). Thus, the term “oligonucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching.

Oligonucleotides may optionally include one or more non-standard nucleotide(s), nucleotide analog(s) and/or modified nucleotides.

In some cases, nucleotide sequences are provided using character representations recommended by the International Union of Pure and Applied Chemistry (IUPAC) or a subset thereof. IUPAC nucleotide codes used herein include, A=Adenine, C=Cytosine, G =Guanine, T=Thymine, U=Uracil, R=A or G, Y=C or T, S=G or C, W=A or T, K=G or T, M=A or C, B=C or G or T, D=A or G or T, H=A or C or T, V=A or C or G, N =any base, “.” or “-”=gap. In some embodiments the set of characters is (A, C, G, T, U) for adenosine, cytidine, guanosine, thymidine, and uridine respectively. In some embodiments the set of characters is (A, C, G, T, U, I, X, ψ) for adenosine, cytidine, guanosine, thymidine, uridine, inosine, uridine, xanthosine, pseudouridine respectively. In some embodiments the set of characters is (A, C, G, T, U, I, X, ψ, R, Y, N) for adenosine, cytidine, guanosine, thymidine, uridine, inosine, uridine, xanthosine, pseudouridine, unspecified purine, unspecified pyrimidine, and unspecified nucleotide respectively.

The terms “staple strands” or “helper strands” are used interchangeably. When used in the context of a nucleic acid nanostructure object, “Staple strands” or “helper strands” refer to oligonucleotides that work as glue to hold the scaffold nucleic acid in its three-dimensional geometry. Additional nucleotides can be added to the staple strand at either 5′ end or 3′ end, and those are referred to as “staple overhangs”. Staple overhangs can be functionalized to have desired properties such as a specific sequence to hybridize to a target nucleic acid sequence, or a targeting element. Target nucleic acid sequences used to mask staple overhangs during the functionalization process are herein referred to as “guard strands”. In some instances, the staple overhang is biotinylated for capturing the DNA nanostructure on a streptavidin-coated bead. In some instances, the staple overhang can be also modified with chemical moieties. Non-limiting examples include CLICK-chemistry groups (e.g., azide group, alkyne group, DIBO/DBCO), amine groups, and thiol groups. In some instances, some bases located inside the oligonucleotide can be modified using base analogs (e.g., 2-Aminopurine, Locked Nucleic Acids, such as those modified with an extra bridge connecting the 2′ oxygen and 4′ carbon) to serve as linker to attach functional moieties (e.g., lipids, proteins). Alternatively, DNA-binding proteins or guide RNAs can be used to attach secondary molecules to the DNA scaffold.

The terms “scaffolded origami”, “origami”, “nucleic acid nanoparticle”, “nucleic acid nanostructure”, “nanostructure”, “nucleic acid assembly” are used interchangeably. They can be one or more short single strands of nucleic acids (staple strands) (e.g., DNA) that fold a long, single strand of polynucleotide (scaffold strand) into desired shapes on the order of about 10 nm to a micron, or more. Wireframe scaffolded DNA origami may use edges having 2. 4, 6, or more duplexes crosslinked in parallel to endow rigidity to the nanoparticle (Jun et al., ACS Nano, 2019, 10.1021/acsnano.8b08671; Veneziano et al., Science, 352(6293):1534 (2016)). Single-stranded DNA scaffold may be produced from M1.3 or using a helper plasmid as shown by Shepherd, et al., bioRxiv 521443 (2019), doi: https://doi.org/10.1101/521443 and Praetorius et al., Nature, 552:84-87 (2017). Alternatively, single-stranded synthetic nucleic acid can fold into an origami object without helper strands, for example, using parallel or paranernic crossover motifs. Alternatively, purely staple strands can form nucleic acid memory blocks of finite extent. The scaffolded origami or origami can be composed of deoxyribonucleotides (DNA) or ribonucleotides (RNA), or analogs or modified nucleotides thereof, including, but not limited to locked nucleic acids (LNA) and peptide nucleic acids (PNA). A scaffold or origami composed of DNA can be referred to as, for example a scaffolded DNA origami or DNA origami, etc. It will be appreciated that where compositions, methods, and systems herein are discussed or exemplified with DNA (e.g., DNA origami), other nucleic acid molecules can be substituted.

The term “polyhedron” refers to a three-dimensional solid figure in which each side is a flat surface. These flat surfaces are polygons and are joined at their edges.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

The terms “epitope” or “antigenic determinant” refer to a site on an antigen to which B and/or T cells respond. In the context of a polypeptide, B-cell epitopes can be formed both from contiguous amino acids or noncontiguous amino acids juxtaposed by tertiary or quarternary folding of a protein. Epitopes formed from contiguous amino acids are typically retained on exposure to denaturing solvents whereas epitopes formed by tertiary or quarternary folding are typically lost on treatment with denaturing solvents. An epitope typically includes at least 3, and more usually, at least 5 or 8-10, amino acids, in a unique spatial conformation. Methods of determining spatial conformation of epitopes include, for example, X-ray crystallography and multi-dimensional nuclear magnetic resonance spectroscopy.

The term “immunostimulatory agent” refers to any agent that stimulates, up-regulates, induces, enhances or otherwise activates one or more physiological pathways associated with the active, passive, innate or adaptive immune response in a subject. Exemplary immunostimulatory agents include antigens, adjuvants, and agonists/ligands for Toll Like Receptor (TLR), T Cell Receptor (TCR), B Cell Receptor (BCR), cytokines, etc.

The term “antigen” as used herein is defined as a molecule capable of being recognized or bound by an antibody, B-cell receptor or T-cell receptor. An “immunogen” is an antigen that is additionally capable of provoking an immune response against itself (e.g., upon administration to a mammal, optionally in conjunction with an adjuvant). This immune response can involve either antibody production, or the activation of specific immunologically-competent cells, or both. Any macromolecule, including virtually all proteins or peptides as well as lipids and oligo- and polysaccharides, can serve as an antigen or immunogen. Furthermore, antigens/immunogens can be derived from recombinant or genomic DNA. Any DNA that includes a nucleotide sequences or a partial nucleotide sequence encoding a protein or peptide that elicits an immune response therefore encodes an “immunogen” as that term is used herein. An antigen/immunogen need not be encoded solely by a full-length nucleotide sequence of a gene. An antigen/immunogen need not be encoded by a “gene” at all. An antigen/immunogen can be generated, synthesized, or can be derived from a biological sample. Such a biological sample can include, but is not limited to a tissue sample, a tumor sample, a cell or a biological fluid.

The term “small molecule,” as used herein, generally refers to an organic molecule that is less than about 2,000 g/mol in molecular weight, less than about 1,500 g/mol, less than about 1,000 g/mol, less than about 800 g/mol, or less than about 500 g/mol. Small molecules are non-polymeric and/or non-oligomeric.

As used herein, the term “carrier” refers to an organic or inorganic ingredient, natural or synthetic, with which the active ingredient is combined to facilitate the application.

As used herein, the term “pharmaceutically acceptable” describes a material that is not biologically or otherwise undesirable. The term refers to those compounds, materials, compositions, and/or dosage forms which are, within the scope of sound medical judgment, suitable for use in contact with the tissues, organs, and/or bodily fluids of a subject without excessive toxicity, irritation, allergic response, or other problems or complications commensurate with a reasonable benefit/risk ratio. Such materials can be administered to a subject along with the selected compound without causing any undesirable biological effects or interacting in a deleterious manner with any of the other components of the pharmaceutical composition in which it is contained.

As used herein, the term “pharmaceutically-acceptable carrier” means one or more compatible solid or liquid fillers, dilutants or encapsulating substances which are suitable for administration to a human or other vertebrate animal.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

“Treatment” or “treating” means to administer a composition to a subject or a system with an undesired condition (e.g., an infectious disease, cancer). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

As used herein, the terms “effective amount” or “therapeutically effective amount” are used interchangeably and mean a quantity sufficient to alleviate or ameliorate one or more symptoms of a disorder, disease, or condition being treated, to induce or enhance an immune response, or to otherwise provide a desired pharmacologic and/or physiologic effect. Such amelioration only requires a reduction or alteration, not necessarily elimination. The precise quantity will vary according to a variety of factors such as subject-dependent variables (e.g., age, immune system health, weight, etc.), the disease or disorder being treated, the disease stage, as well as the route of administration, and the pharmacokinetics and pharmacodynamics of the agent being administered.

Recitation of ranges of values herein are merely intended to serve as a shorthand method of referring individually to each separate value falling within the range, unless otherwise indicated herein, and each separate value is incorporated into the specification as if it were individually recited herein.

### II. Compositions

Peptide- and protein-based vaccines form the major class of vaccination strategies globally. Inactivated viral particles or other passivated protein nanoparticles can be used in vaccine formulations to display protein or peptide antigens to stimulate and train the immune response. Adjuvants including small molecules and nucleic acids are often co-formulated with these antigens to further stimulate immune activation.

Disclosed herein is a highly versatile class of viral-like nanoparticle formed of structured nucleic acid structures displaying immunostimulatory agents. One or more immunostimulatory agents of interest can be displayed in varying copy numbers with precise control over inter-antigen spacing, number, and spatial organization in 1, 2, and 3 dimensions.

The disclosed viral-like particles are based in part on the generation and testing of DNA origami nanoparticles having four icosahedral nanoparticles with diameters of 30 or 40 nm displaying 30 or 60 copies of eOD-GT8 (see Example 8). These DNA origami nanoparticles were used to investigate the effects of antigen valency and spacing on vaccine trafficking in vivo and germinal center formation after a prime immunization. As shown in the Examples below, all the nanoparticles effectively activated cognate germline VRC01 Ramos B cells in vitro. As antigen density increased, an increase in deposition of mannose binding lectin and C3 was observed in mouse serum in vitro, and this correlated with improved follicular targeting in vivo. Specifically, only the design with the highest antigen density (diameter: 30 nm, valency: 60 eOD-GT8 antigens) successfully enhanced germinal center responses compared to soluble monomer. Also, these DNA nanoparticles led to a 15-fold improvement in the frequency of antigen-specific GC B cells compared to an equivalent dose of monomer, and 3-fold increase compared to a clinical trial candidate eOD-GT8 60mer, which is formulated on a protein scaffold. The Examples also demonstrate incorporation of a universal helper T cell epitope (PADRE) fused to eOD-GT8, which further elevated the overall number of antigen-specific germinal center B cells and follicular helper T cells.

Immunodominance and B cell competition in germinal centers create a challenging environment for the selection and survival of rare, low affinity B cells clones, such as VRCO1-class precursors¹⁹. In contrast to protein nanoparticles such as the eOD-60mer, where antigen-specific and scaffold-specific B cells compete for the same helper T cells (due to presentation of scaffold-derived peptides on both antigen- and scaffold-specific B cells), GCs formed by DNA-VLPs should experience reduced B cell competition due to the T-independent nature of the scaffold. Furthermore, incorporating synthetic T cell helper epitopes into DNA-VLP formulations provides T cell help only to antigen-specific B cells, which may boost the antigen-specific composition of germinal centers. The disclosed DNA-VLP nanoparticles offer precise nanoscale antigen organization on inert scaffolds with focused T cell help for enhancing germinal center responses post-prime and minimizing unwanted competitor B cell responses.

Protein/peptide-conjugation strategies based on peptide nucleic acids are provided and can be used to adapt the platform for display of any number of immunostimulatory agent, such as antigens, adjuvants, or combinations thereof. The nucleic acid sequence of the underlying viral-like nanostructure scaffold can be controlled fully together with adjuvant display of small molecules or targeting ligands including sugars. Chemical modifications to the DNA nanoparticle including 3′ and 5′ PEGylation and incorporation of phosphorothioates may be used to stabilize and passivate nanostructures for in vivo administration. Additionally, or alternatively, 3′ and/or 5′ terminal ends of staples may be ligated to stabilize the nanoparticle from exonuclease degradation. Cationic polymers and minor groove binders (as monomers, oligomers or polymers) may be used to coat the DNA nanoparticles for stabilization from endonuclease degradation. In particular, minor groove binders may act as tethers for covalent modifications of nucleic acids, for example to develop cross-linking strategies for DNA nanoparticles. These approaches can be combined, e.g. using brush or block copolymers, with PEGylation to further improve stabilization and passivation. Exemplary DNA nanoparticles for displaying antigens and adjuvants are further described in U.S. application Ser. Nos. 17/819,204 and 16/752,394, U.S. Provisional Application No. 62/796,472, U.S. Provisional Application No. 63/333,498, all of which are hereby incorporated by reference in their entireties.

For example, HIV presents distinct barriers against the formation of prophylactic immune responses due to antigenic diversity of viral proteins as well as the similarity of the virus to self-epitopes (Burton, et al., Science, 337(6091):183-186 (2012); Burnett, et al., Science, 360(6385):223-226 (2018)). The HIV gp120 CD4 binding site protein has emerged as an attractive target for the induction of broadly neutralizing antibodies against HIV. Env spike proteins are sparsely distributed on the viral surface, possibly allowing HIV to evade host immune responses (Zhu, et al., Nature, 441(7095):847-852 (2006); Klein, J S. and Bjorkman, PJ., PLOS Pathog., 6(5):e1000908 (2010)). Consistent with this observation, germline-targeting CD4 binding-site immunogens assembled into protein nanoparticles have allowed for highly multivalent (e.g., 60mer) antigen presentation to B cells. It is thought that such high degree of multimerization of immunogens on nanoparticles elicits enhanced adaptive immune responses (Jardine, et al., Science, 349(6244):156-161 (2015), Abbott, et al., Immunity, 48(1):133-146.e6 (2018)). However, it was previously unknown how the structural features of nanoparticle-antigen presentation (such as for example, antigen number, inter-antigen distance, 3D organization) and how scaffold-specific B cells responses affect the priming and maturation of relevant bnAb lineages.

In the Examples described below, eOD-GT8 antigens were displayed on DNA origami, systematically varying antigen number, inter-antigen distances, and 3D organization. The Examples show that eOD dimers templated by DNA origami elicited robust B cell responses in vitro at inter-antigen distances greater than 28 nm. These results indicate that sparse distributions of viral spikes on HIV and other viruses do not inhibit B cell receptor signaling responses, and support non-local models for B cell receptor activation mechanisms. The Examples also demonstrate that higher density viral like particles formed from DNA origami (DNA-VLPs) i.e., by increase the number of antigens (more than -30 antigens) and reducing the inter-antigenic distance (less than ˜15 nm) results in increased recruitment of high-mannose glycans for triggering the mannose binding lectin pathway and follicle targeting.

Hence, the distances between antigenic sites are important determinants of B cell receptor activation and cellular response. In contrast to a model where tight clustering of antigen sites yields greater B cell activation due to spatially-dependent cooperative effects between B cell receptor immunoglobulin signaling subunits, the Examples show that extended antigen placement led to equivalent, and in some cases superior, B cell receptor activation.

Although compositions and methods of use for treatment of HIV are expressly provided and exemplified in the experiments below, the illustrated principles are believed to extend to wide-ranging compositions and methods of immune modulation by varying, for example, the antigen(s) and/or nanostructure(s) and in some cases further varying the valance and spatial organization of the antigen(s). Unlike biologically produced vaccine particles, fully synthetic production of the entire platform offers strict quality control over the formulation. The platform can be utilized in immune stimulation as well as immune tolerance using proteins, peptides, and small molecule adjuvants. Targeting molecules can be used to direct the compositions to desired tissues or cells. Methods of using the compositions for treating infectious diseases, auto-immune diseases, as well as in cancer immunotherapies are also provided.

The size of the DNA-VLP nanoparticles can range from about 15 nm to about 40 nm in diameter. In some forms, the DNA-VLP can have diameters ranging from about 20 nm to about 35 nm, 23 nm to about 34 nm, 25 nm to about 30 nm, 25 nm to about 30 nm. For example, the DNA-VLP can have a diameter of about 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, or 40 nm.

The disclosed compositions typically include one or more immunostimulatory agents such as antigens or adjuvants bound or linked to, incorporated into (e.g., bound to surfaces of, encapsulated in), or otherwise associated with, nucleic acid nanostructures. The antigens, adjuvants, and other immunostimulatory agents are typically displayed on the nanostructure surface and can be arranged for their most preferred presentation (e.g., preferred valency, preferred spacing, preferred rigidity/flexibility, preferred dimensionality, etc.) for eliciting an immune response.

Exemplary immunostimulatory agents such as antigens and adjuvants and nanostructures are each discussed in more detail below.

### A. Immunostimulatory Agents

Nanostructures including one or more immunostimulatory agents are provided. Exemplary immunostimulatory agents are capable of activating, stimulating, inducing or otherwise actuating one or more pathways associated with one or more of the active, passive, adaptive or innate immune systems in a subject are provided. In some embodiments, the immunostimulatory agent is an antigen or an adjuvant. In some embodiments, the immunostimulatory agent is a ligand for a cell-bound receptor, such as a Toll like receptor. Examples of immunostimulatory agents are discussed below and elsewhere herein.

### 1. Antigens

In some forms, the immunostimulatory agent is an antigen. Antigens are compounds that are specifically bound by antibodies or T lymphocyte antigen receptors. They stimulate production of or are recognized by antibodies. Sometimes antigens are part of the host itself in an autoimmune disease. An immunogen is an antigen (or adduct) that is able to trigger a humoral or cell-mediated immune response. It first initiates an innate immune response, which then causes the activation of the adaptive immune response. An antigen binds the highly variable immunoreceptor products (B cell receptor or T cell receptor) once these have been generated. Immunogens are those antigens, termed immunogenic, capable of inducing an immune response. Thus, an immunogen is necessarily an antigen, but an antigen may not necessarily be an immunogen. For brevity, the disclosed nanostructure-based compositions are typically referred to as having an antigen conjugated or bound thereto or otherwise associate therewith. However, unless specifically indicated otherwise, any of the antigens can also be an immunogenic (i.e., an immunogen). Thus, all the disclosure of compositions and methods of use related to antigen bound nanostructures is also expressly provided with respect to immunogen bound nanostructures unless indicated to the contrary.

As discussed in more detail below, in some embodiments, antigens are selected or designed for immune stimulation or immune tolerance, of B-cells and/or T-cells, with or without the context of an MHC complex. In some embodiments, the antigen-bound nanostructures mimic dendritic cell presentation of antigenic peptides. Such embodiments may include MHC complex presentation of antigen(s) incorporated into the nanostructure.

The nucleic acid nanostructures act as scaffolds for one or more copies of one or more antigens. The nanostructures can be used to capture and/or restrain the antigen in a fixed and known orientation, for example, in preferred antigen presentation for eliciting an immune response. For example, organizations (e.g., 1D, 2D, 3D) of viral proteins can be used to stimulate the immune system by presenting these proteins in geometries that mimic the one or more naturally occurring antigens, or in geometries designed to elicit a robust immune response.

The nucleic acid nanostructures allow for control of the relative position, number, flexibility or rigidity, and/or dimensionality of the antigen that it contains (e.g., bound to its surface).

It is believed there is virtually no limit to the number of copies of antigen, or number of different (types of) antigens, beyond any structural limitations of the nanostructure itself, which can also be increased in size and complexity to accommodate increasing numbers of antigen. For example, the nanostructure can be functionalized with any integer number of antigens from 1 to 1000, or any specific range of there between. A nanostructure can include, e.g., between about 1 and 100, or 2 and 60, or 3 and 50, or 4 and 25, or 5 and 10 antigen molecules. A nanostructure can have 2, 3, 4, 5, 6, 7, 8, 9, 10, 11, 12, 13, 14, 15, 16, 17, 18, 19, 20, 25, 30, 35, 40, 45, 50, 55, 60, etc. antigens. In particular embodiments, a nanostructure has 5-10 antigen molecules (e.g., 5, 6, 7, 8, 9, or 10 copies). Large structures, e.g., microstructures, may include hundreds of antigens.

The Examples below demonstrate that some in vivo B cell responses increase with increasing copies of antigen (eOD-GT8) between about 30 and about 60 copies of antigen. Thus, in some embodiments, the number of copies of an antigen is between 1 and 1000, 1 and 800, preferably, between 30 and 800, or between 40 and 70, inclusive, particularly where the nanostructure is icosahedral.

As used herein, “adjacent antigen” can refer to the antigen or antigens in closest proximity to a reference antigen. In particular embodiments, the adjacent antigen must be on the same face of the nanostructure. In some embodiments, there will be two or more adjacent antigens to a single reference antigen. Each adjacent antigen can independently be another copy or copies of the reference antigen or a structurally different antigen or antigens from the reference antigen. Thus, in some embodiments, all of the adjacent antigens are structurally the same as the reference antigen, all of the adjacent antigens are structurally different from the reference antigen, or the adjacent antigens are a combination of being structurally the same and structurally different from the reference antigen. Antigens may be covalently or non-covalently attached to the nanostructure, and they may be cleavable by proteases or other enzymes or undergo triggered dissociation in response to environmental cues such as pH, etc. They and/or the nanoparticle may also be shielded from the immune system by encapsulating polymers or other materials for shielding and targeting purposes prior to antigen exposure at physiological sites of interest such as the injection site or within lymph nodes.

The inter-antigen distance between any two adjacent antigens can be any integer number from 1 nm to 500 nm, or any specific range there between. For example, in some embodiments, the distance between two adjacent antigens, also referred to herein as inter-antigen distance and the space between two antigens, is in the range of 1 nm to 150 nm, or 10 nm to 100 nm, or 15 nm to 80 nm, or 28 nm to 80 nm, or 10 nm to 80 nm, or 15 nm to 50 nm, or 25 nm to 30 nm, or 2 nm and 10 nm, or 2 nm and 8 nm, or 2 nm and 6 nm, or 2 nm and 4 nm. The Examples below show that B cell responses increased when inter-antigen distance (eOD-GT8) was less than 28 nm when presented on a 6HB dimer, or 15 nm when presented on a polyhedron. Thus, preferably, the distance between two adjacent antigens is at least 2 nm, or at least 6 nm. The Examples did not illustrate a decrease in B cell responses when the distance increased beyond 80 nm, a distance well beyond the size where two separate immunoglobulin Iga/P pairs could be interacting. However, in some embodiments, the inter-antigen distance does not exceed the distance where two separate immunoglobulin Iga/P pairs could be interacting.

In some specific embodiments, the distance between adjacent antigens is 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 100, 150, 200, 25, 300, 400, or 500 nm.

The distance between adjacent antigens may vary based on other parameters, such as, the shape of the nanostructure being used. For example, in some embodiments, when the nanostructure is a 6HB, the distance between adjacent antigens can be 28 nm or more (e.g., 28, 29, 30, 35, 40, 45, 50, 55, 60, 65, 70, 75 nm, or more). In some embodiments, when the nanostructure is a polyhedron, such as an icosahedron for example, the distance between adjacent antigens (e.g., on the same face of the nanostructure) are 2 nm or more (e.g., 2, 3, 4, 5, 6,7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75 nm, or more).

The number, size and amount of immunostimulatory agents, such as antigens or immunogens, that are conjugation to the described nanostructures is typically sufficient to provide a specific surface coverage across the exposed surface(s) of the nanostructure. For example, in some forms, the number, size and amount of individual molecules of an immunostimulatory agent, such as a peptide, oligosaccharide, small molecule, etc., can occlude some or all of the exposed surface area(s) of a described nanostructure. In some forms, the coverage is described as a function of the % of the surface that is occupied or otherwise occluded by the molecules of immunostimulatory agent(s). In some forms, the surface density is presented as a minimum surface density value that is required to achieve a given biological or physiological function. An exemplary minimum surface density is between about 0.1 to 1 molecule/nm². The minimum surface density required to achieve a biological function can vary, for example, corresponding to the size and/or composition of a nanostructure, or immunostimulatory agent(s) conjugated thereto. In other forms, the surface conjugation of a plurality of immunogens can be expressed as a maximum inter-antigen distance that is necessary or sufficient to achieve a specific biological function in vivo. For example, in some forms, the distance between two molecules of the plurality of antigen molecules on the NAN surface is between about 10 nm and about 4 nm, inclusive.

The Examples below demonstrate that an antigen density of 0.016 antigen/nm², corresponding to a glycan density of 0.136 glycan patch/nm² (240 glycan patches/structure) and inter-antigen spacing of 4-6 nanometers, resulted in superior delivery to lymph nodes and activation of antigen-specific GC B cells in vivo.

Antigen location and orientation can also be varied. For example, antigen placement can be linear, or clustered. Inter-antigen distance between adjacent antigens can be equidistant or non-equidistant. Inter-antigen distance can be equidistant in 2, 3, 4, 5 or more directions. The Examples show that for larger inter-antigen distances (e.g., 15 nm or greater), clustering on both linear and planar structures leads to an equivalent cellular response. However, when inter-antigen distances are smaller (e.g., less than 14 nm), linear placement of antigen yields a greater response than a clustered planar placement of antigen. In both cases, further decreases can cause a relative reduction in immune response.

The antigen can be covalently or non-covalently bound to the nanostructure. In some embodiments, the antigen is directly or indirectly bound to the nanostructure via outwardly facing nucleic acid overhangs extending from the 3′ and/or 5′ ends of selected staple strands. The exemplary embodiments below feature 3′ overhangs. The nucleic acid overhangs can include one or more sequences that is complementary to a target RNA, DNA or PNA sequence. In some embodiments, the nucleic acid overhangs hybridize to the complementary target RNA, DNA or PNA sequence which can be covalently linked to the antigen. Such covalent linkage can be formed by maleimide-thiol coupling. Some exemplary non-covalent interactions for attachment or incorporation include intercalation, biotin- streptavidin interaction, or hybridization between complementary nucleotide sequences. In situ template CLICK chemistry can also be used to covalently attach the antigen to the nucleic acid nanoparticle following a non-covalent, hybridization reaction that templates the antigen by binding it to the nanoparticle through PNA:DNA or other nucleic acid hybridization reaction. In a preferred embodiment, copper-assisted alkyne-azide CLICK chemistry, copper-free strain promoted azide-alkyne cycloaddition, or other catalyst-dependent bioconjugate techniques are used for the implementation of this approach by the addition of the catalyst after removal of excess antigen to maintain overhang sequence-specific addressability of the DNA nanoparticle.

Antigens can be or can include, for example, proteins, nucleic acids, lipids, and oligo- or polysaccharides as well as the corresponding cooligo- and polymers. Exemplary antigens include B cell antigens and T cell antigens. B cell antigens can be peptides, proteins, oligo- and polysaccharides, lipids, nucleic acids, small molecules (alone or with a hapten) or combinations thereof. T cell antigens are typically proteins or peptides. The antigen can be derived from a virus, bacterium, parasite, plant, protozoan, fungus, tissue or transformed cell such as a cancer or leukemic cell and immunogenic component thereof, e.g., cell wall components or molecular components thereof. The antigens can be allergens or environmental antigens or tumor antigens. The antigen can be associated with one or more diseases or conditions such as infectious diseases, autoimmune diseases, and cancer. In some forms, the antigen is derived from endogenous protein(s) in humans generally, or from a patient in particular. For example in some forms, the antigen is an autologous antigen. In some forms, the antigen is derived from a naturally-occurring or synthetic small molecule(s).

Suitable antigens are known in the art and are available from commercial, government and scientific sources. The antigens can be purified or partially purified polypeptides derived from tumors or viral or bacterial sources. The antigens can be recombinant polypeptides produced by expressing DNA encoding the polypeptide antigen in a heterologous expression system. Antigens can be provided as single antigens or can be provided in combination. Antigens can also be provided as complex mixtures of polypeptides or nucleic acids.

In some embodiments, the antigen is a viral antigen. A viral antigen can be isolated from any virus. In an exemplary embodiment, the antigen is a natural viral capsid structure. In some embodiments, the antigen is a bacterial antigen. Bacterial antigens can originate from any bacteria. In some embodiments the antigen is a parasite antigen. In some embodiments, the antigen is an allergen or environmental antigen. Exemplary allergens and environmental antigens, include but are not limited to, an antigen derived from naturally occurring allergens such as pollen allergens (tree-, herb, weed-, and grass pollen allergens), insect allergens (inhalant, saliva and venom allergens), animal hair and dandruff allergens, and food allergens. In some embodiments, the antigen is a self-antigen such as in immune tolerance applications for auto-immune or related disorders such as Multiple Sclerosis. In some embodiments, the antigen is a tumor antigen. Exemplary tumor antigens include a tumor-associated or tumor-specific antigen. In some forms, the antigen is a peptide that stimulates a humoral immune response against an endogenous protein. In some forms, the antigen is a small molecule that stimulates a humoral immune response against an exogenous small molecule.

In some embodiments, the antigen is a peptide derived from the H-2Kk MHC class I molecule. For example, a nucleic acid nanostructure having a defined geometric shape and one or more copies of an antigen than can induce a response from B cell specific to the H-2K^{k} MHC class I molecule are provided. In some embodiments, the antigens have been previously defined and are known to have varying affinity B cell receptors from NOD.D2(B10)-Tg(Igh2^{k}3-83)1Nemz/Dvs mice (Kouskoff, et al, Journal of Experimental Medicine, 188(8):1453-64, 1998).

In particular embodiments, the antigen is the p31 peptide, which has a high affinity for NOD.D2(B10)-Tg(Igh2^{k}3-83)1Nemz/Dvs B cell receptors, or the p5 peptide, which has an intermediate affinity for NOD.D2(B10)-Tg(Igh2^{k}3-83)1Nemz/Dvs B cell receptors, placed on, for example, a pentagonal bipyramidal structure.

i. Viral antigens

A viral antigen can be isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue DINS1, Dengue D1NS2, and Dengue D1NS3.

Viral antigens can be derived from a particular strain such as a papilloma virus, a herpes virus, e.g., herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomegalovirus, Epstein-Barr, rotavirus, rhinovirus, adenovirus, coxsackieviruses, equine encephalitis, Japanese encephalitis, yellow fever, Rift Valley fever, and lymphocytic choriomeningitis.

### (a) HIV Immunogens

In some forms, the antigen is an HIV antigen. The antigen may bind to a broadly neutralizing antibody. The antigen can be, for example, an HIV gp120 epitope of an HIV envelope trimer. In some forms, the antigen is an HIV epitope that encompasses all or part of a gp120 protein derived from an HIV virus. In some forms, the part of a gp120 protein includes a known binding site for a T cell receptor. For example, in some embodiments, the binding site is a CD4 binding site. Exemplary antigens include, but are not limited to, eOD-GT6, eOD-GT8, p5, p31, and/or variants thereof. In another form, the antigen is a core-g28v2 immunogen. Exemplary core-g28v2 peptides and mRNAs are described in Cottrell et al, Sci Transl Med. 2024 May 22;16(748):eadn0223. doi: 10.1126/scitranslmed.adn0223. Epub 2024 May 22. PMID: 38753806; PMCID: PMC11233128.

In some forms, an HIV immunogen is an engineered immunogen. An exemplary immunogen includes engineered outer domain (eOD), core-g28v2, or SOSIP trimer. In other forms, an HIV immunogen is a polypeptide or protein that is designed, through computational and/or experimental methods, to stimulate the germline B cell precursor of a broadly neutralizing antibody class. Example classes include the VRC01, VRC03, 3BNC117, N6, CH103, PG9, PG16, CHO1-CH04, PGT145, 10-1074, PGT121, PGT128, and BG18. In other forms, an HIV immunogen is an engineered polypeptide that is designed, through computational and/or experimental methods, to stimulate B cells that have been previously stimulated by a previous immunization.

ii. Bacterial Antigens

Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptospirosis, Listeria, Meningococcus A, B and C, Methanobacterium, Micrococcus, Mycobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

iii. Parasite Antigens

Parasite antigens can be obtained from parasites such as, but not limited to, an antigen derived from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

iv. Allergens and Environmental Antigens

The antigen can be an allergen or environmental antigen, such as, but not limited to, an antigen derived from naturally occurring allergens such as pollen allergens (tree-, herb, weed-, and grass pollen allergens), insect allergens (inhalant, saliva and venom allergens), animal hair and dandruff allergens, and food allergens. Important pollen allergens from trees, grasses and herbs originate from the taxonomic orders of Fagales, Oleales, Pinales and platanaceae including i.a. birch (Betula), alder (Alnus), hazel (Corylus), hornbeam (Carpinus) and olive (Olea), cedar (Cryptomeria and Juniperus), Plane tree (Platanus), the order of Poales including e.g., grasses of the genera Lolium, Phleum, Poa, Cynodon, Dactylis, Holcus, Phalaris, Secale, and Sorghum, the orders of Asterales and Urticales including i.a. herbs of the genera Ambrosia, Artemisia, and Parietaria. Other allergen antigens that may be used include allergens from house dust mites of the genus Dermatophagoides and Euroglyphus, storage mite e.g. Lepidoglyphus, Glycyphagus and Tyrophagus, those from cockroaches, midges and fleas e.g. Blatella, Periplaneta, Chironomus and Ctenocepphalides, those from mammals such as cat, dog and horse, birds, venom allergens including such originating from stinging or biting insects such as those from the taxonomic order of Hymenoptera including bees (superfamily Apidae), wasps (superfamily Vespidea), and ants (superfamily Formicoidae). Still other allergen antigens that may be used include inhalation allergens from fungi such as from the genera Alternaria and Cladosporium.

v. Cancer Antigens A cancer antigen is an antigen that is typically expressed preferentially by cancer cells (i.e., it is expressed at higher levels in cancer cells than on non-cancer cells; cancer-associated antigen) and in some instances it is expressed solely by cancer cells (cancer-specific antigen). The cancer antigen may be expressed within a cancer cell or on the surface of the cancer cell. The cancer antigen can be MART-1/Melan-A, gp100, adenosine deaminase-binding protein (ADAbp), FAP, cyclophilin b, colorectal associated antigen (CRC)-C017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A1l, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-1, GAGE-2, GAGE-3, GAGE-4, GAGE-5, GAGE-6, GAGE-7, GAGE-8, GAGE-9, BAGE, RAGE, LAGE-1, NAG, GnT-V, MUM-1, CDK4, tyrosinase, p53, MUC family, HER2/neu, p2iras, RCAS1, α-fetoprotein, E-cadherin, α-catenin, β-catenin, γ-catenin, p120ctn, gp100Pmeli17, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, lmp-1, PiA, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, or c-erbB-2.
vi. Neoantigens

In some embodiments the antigen is a neoantigen or a patient-specific antigen.

Recent technological improvements have made it possible to identify the immune response to patient-specific neoantigens that arise as a consequence of tumor-specific mutations, and emerging data indicate that recognition of such neoantigens is a major factor in the activity of clinical immunotherapies (Schumacher and Schreidber, Science, 348(6230):69-74 (2015). Neoantigen load provides an avenue to selectively enhance T cell reactivity against this class of antigens.

Traditionally, cancer vaccines have targeted tumor-associated antigens (TAAs) which can be expressed not only on tumor cells but in the normal tissues (Ito, et al., Cancer Neoantigens: A Promising Source of Immunogens for Cancer Immunotherapy. J Clin Cell Immunol, 6:322 (2015) doi:10.4172/2155-9899.1000322). TAAs include cancer-testis antigens and differentiation antigens, and even though self-antigens have the benefit of being useful for diverse patients, expanded T cells with the high-affinity TCR (T-cell receptor) needed to overcome the central and peripheral tolerance of the host, which would impair anti-tumor T-cell activities and increase risks of autoimmune reactions.

Thus, in some embodiments, the antigen is recognized as “non-self” by the host immune system, and preferably can bypass central tolerance in the thymus. Examples include pathogen-associated antigens, mutated growth factor receptor, mutated K-ras, or idiotype-derived antigens. Somatic mutations in tumor genes, which usually accumulate tens to hundreds of fold during neoplastic transformation, could occur in protein-coding regions. Whether missense or frameshift, every mutation has the potential to generate tumor-specific antigens. These mutant antigens can be referred to as “cancer neoantigens” Ito, et al., Cancer Neoantigens: A Promising Source of Immunogens for Cancer Immunotherapy. J Clin Cell Immunol, 6:322 (2015) doi:10.4172/2155-9899.1000322. Neoantigen-based cancer vaccines have the potential to induce more robust and specific anti-tumor T-cell responses compared with conventional shared-antigen-targeted vaccines. Recent developments in genomics and bioinformatics, including massively parallel sequencing (MPS) and epitope prediction algorithms, have provided a major breakthrough in identifying and selecting neoantigens.

vii. Tolerogenic Antigens

In some cases, the tolerogenic antigen is derived from a therapeutic agent protein to which tolerance is desired. Examples are protein drugs in their wild type, e.g., human factor VIII or factor IX, to which patients did not establish central tolerance because they were deficient in those proteins; or nonhuman protein drugs, used in a human. Other examples are protein drugs that are glycosylated in nonhuman forms due to production, or engineered protein drugs, e.g., having non-native sequences that can provoke an unwanted immune response. Examples of tolerogenic antigens that are engineered therapeutic proteins not naturally found in humans including human proteins with engineered mutations, e.g., mutations to improve pharmacological characteristics. Examples of tolerogenic antigens that have nonhuman glycosylation include proteins produced in yeast or insect cells.

Tolerogenic antigens can be from proteins that are administered to humans that are deficient in the protein. Deficient means that the patient receiving the protein does not naturally produce enough of the protein. Moreover, the proteins can be proteins for which a patient is genetically deficient. Such proteins include, for example, antithrombin-III, protein C, factor VIII, factor IX, growth hormone, somatotropin, insulin, pramlintide acetate, mecasermin (IGF-1), 3-gluco cerebrosidase, alglucosidase-.alpha., laronidase (α-L-iduronidase), idursuphase (iduronate-2-sulphatase), galsulphase, agalsidase-.beta. (α-galactosidase), α-1 proteinase inhibitor, and albumin.

The tolerogenic antigen can be from therapeutic antibodies and antibody-like molecules, including antibody fragments and fusion proteins with antibodies and antibody fragments. These include nonhuman (such as mouse) antibodies, chimeric antibodies, and humanized antibodies. Immune responses to even humanized antibodies have been observed in humans (Getts D R, Getts M T, McCarthy D P, Chastain E M L, & Miller S D (2010), mAbs, 2(6):682-694).

The tolerogenic antigen can be from proteins that are nonhuman. Examples of such proteins include CRISPR-associated proteins, adenosine deaminase, pancreatic lipase, pancreatic amylase, lactase, botulinum toxin type A, botulinum toxin type B, collagenase, hyaluronidase, papain, L-Asparaginase, rasburicase, lepirudin, streptokinase, anistreplase (anisoylated plasminogen streptokinase activator complex), antithymocyte globulin, crotalidae polyvalent immune Fab, digoxin immune serum Fab, L-arginase, and L-methionase.

Tolerogenic antigens include those from human allograft transplantation antigens. Examples of these antigens are the subunits of the various MHC class I and MHC class II haplotype proteins, and single-amino-acid polymorphisms on minor blood group antigens including RhCE, Kell, Kidd, Duffy and Ss.

The tolerogenic antigen can be a self-antigen against which a patient has developed an autoimmune response or may develop an autoimmune response. Examples are proinsulin (diabetes), collagens (rheumatoid arthritis), myelin basic protein (multiple sclerosis). For instance, Type 1 diabetes mellitus (T1D) is an autoimmune disease whereby T cells that recognize islet proteins have broken free of immune regulation and signal the immune system to destroy pancreatic tissue. Numerous protein antigens that are targets of such diabetogenic T cells have been discovered, including insulin, GAD65, chromogranin-A, among others. In the treatment or prevention of T1D, it would be useful to induce antigen-specific immune tolerance towards defined diabetogenic antigens to functionally inactivate or delete the diabetogenic T cell clones.

Tolerance and/or delay of onset or progression of autoimmune diseases may be achieved for various of the many proteins that are human autoimmune proteins, a term referring to various autoimmune diseases wherein the protein or proteins causing the disease are known or can be established by routine testing. In some embodiments, a patient is tested to identify an autoimmune protein and an antigen is created for use in a molecular fusion to create immunotolerance to the protein.

Embodiments can include an antigen, or choosing an antigen from or derived from, one or more of the following proteins. In type 1 diabetes mellitus, several main antigens have been identified: insulin, proinsulin, preproinsulin, glutamic acid decarboxylase-65 (GAD-65), GAD-67, insulinoma-associated protein 2 (IA-2), and insulinoma-associated protein 2.beta. (IA-213); other antigens include ICA69, ICA12 (SOX-13), carboxypeptidase H, Imogen 38, GLIMA 38, chromogranin-A, FISP-60, caboxypeptidase E, peripherin, glucose transporter 2, hepatocarcinoma-intestine-pancreas/pancreatic associated protein, S1000, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5. In autoimmune diseases of the thyroid, including Hashimoto's thyroiditis and Graves' disease, main antigens include thyroglobulin (TG), thyroid peroxidase (TPO) and thyrotropin receptor (TSHR); other antigens include sodium iodine symporter (NIS) and megalin. In thyroid-associated ophthalmopathy and dermopathy, in addition to thyroid autoantigens including TSHR, an antigen is insulin-like growth factor 1 receptor. In hypoparathyroidism, a main antigen is calcium sensitive receptor. In Addison's disease, main antigens include 21-hydroxylase, 17α-hydroxylase, and P450 side chain cleavage enzyme (P450scc); other antigens include ACTH receptor, P450c21 and P450c17. In premature ovarian failure, main antigens include FSH receptor and .alpha.-enolase. In autoimmune hypophysitis, or pituitary autoimmune disease, main antigens include pituitary gland-specific protein factor (PGSF) la and 2; another antigen is type 2 iodothyronine deiodinase. In multiple sclerosis, main antigens include myelin basic protein, myelin oligodendrocyte glycoprotein and proteolipid protein. In rheumatoid arthritis, a main antigen is collagen II. In immunogastritis, a main antigen is H+, K+-ATPase. In pernicious angemis, a main antigen is intrinsic factor. In celiac disease, main antigens are tissue transglutaminase and gliadin. In vitiligo, a main antigen is tyrosinase, and tyrosinase related protein 1 and 2. In myasthenia gravis, a main antigen is acetylcholine receptor. In pemphigus vulgaris and variants, main antigens are desmoglein 3, 1 and 4; other antigens include pemphaxin, desmocollins, plakoglobin, perplakin, desmoplakins, and acetylcholine receptor. In bullous pemphigoid, main antigens include BP180 and BP230; other antigens include plectin and laminin 5. In dermatitis herpetiformis Duhring, main antigens include endomysium and tissue transglutaminase. In epidermolysis bullosa acquisita, a main antigen is collagen VII. In systemic sclerosis, main antigens include matrix metalloproteinase 1 and 3, the collagen-specific molecular chaperone heat-shock protein 47, fibrillin-1, and PDGF receptor; other antigens include Scl-70, U1 RNP, Th/To, Ku, Jol, NAG-2, centromere proteins, topoisomerase I, nucleolar proteins, RNA polymerase I, II and III, PM-Slc, fibrillarin, and B23. In mixed connective tissue disease, a main antigen is U1snRNP. In Sjogren's syndrome, the main antigens are nuclear antigens SS-A and SS-B; other antigens include fodrin, poly(ADP-ribose) polymerase and topoisomerase. In systemic lupus erythematosus, main antigens include nuclear proteins including SS-A, high mobility group box 1 (HMGB1), nucleosomes, histone proteins and double-stranded DNA. In Goodpasture's syndrome, main antigens include glomerular basement membrane proteins including collagen IV. In rheumatic heart disease, a main antigen is cardiac myosin. Other autoantigens revealed in autoimmune polyglandular syndrome type 1 include aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and the type 1 interferons interferon alpha, beta and omega. In some cases, the tolerogenic antigen is a foreign antigen against which a patient has developed an unwanted immune response. Examples are food antigens. Some embodiments include testing a patient to identify foreign antigen and creating a molecular fusion that includes the antigen and treating the patient to develop immunotolerance to the antigen or food. Examples of such foods and/or antigens are provided. Examples are from peanut: conarachin (Ara h 1), allergen II (Ara h 2), arachis agglutinin, conglutin (Ara h 6); from apple: 31 kda major allergen/disease resistance protein homolog (Mal d 2), lipid transfer protein precursor (Mal d 3), major allergen Mal d 1.03D (Mal d 1); from milk: .alpha.-lactalbumin (ALA), lactotransferrin; from kiwi: actinidin (Act c 1, Act d 1), phytocystatin, thaumatin-like protein (Act d 2), kiwellin (Act d 5); from mustard: 2S albumin (Sin a 1), 11 S globulin (Sin a 2), lipid transfer protein (Sin a 3), profilin (Sin a 4); from celery: profilin (Api g 4), high molecular weight glycoprotein (Api g 5); from shrimp: Pen a 1 allergen (Pen a 1), allergen Pen m 2 (Pen in 2), tropomyosin fast isoform; from wheat and/or other cereals: high molecular weight glutenin, low molecular weight glutenin, alpha- and gamma-gliadin, hordein, secalin, avenin; from strawberry: major strawberry allergy Fra a 1-E (Fra a 1), from banana: profilin (Mus xp 1). Many protein drugs that are used in human and veterinary medicine induce immune responses, which create risks for the patient and limits the efficacy of the drug. This can occur with human proteins that have been engineered, with human proteins used in patients with congenital deficiencies in production of that protein, and with nonhuman proteins. It would be advantageous to tolerize a recipient to these protein drugs prior to initial administration, and it would be advantageous to tolerize a recipient to these protein drugs after initial administration and development of immune response. In patients with autoimmunity, the self-antigen(s) to which autoimmunity is developed are known. In these cases, it would be advantageous to tolerize subjects at risk prior to development of autoimmunity, and it would be advantageous to tolerize subjects at the time of or after development of biomolecular indicators of incipient autoimmunity. For example, in Type 1 diabetes mellitus, immunological indicators of autoimmunity are present before broad destruction of beta cells in the pancreas and onset of clinical disease involved in glucose homeostasis. It would be advantageous to tolerize a subject after detection of these immunological indicators prior to onset of clinical disease.

### 2. Immunostimulatory Receptor Agonists

In some embodiments, the immunostimulatory agent is a ligand or binding partner for an immunostimulatory receptor. Ligands that bind to receptors to induce activation of one or more physiological pathways through the immunostimulatory receptor are termed immunostimulatory receptor agonists. Therefore, in some embodiments, the immunostimulatory agent is an immunostimulatory receptor agonist. Exemplary immunostimulatory receptors include those associated with activation of the innate immune system.

The innate immune system contains several pattern recognition receptors (PRRs) that are responsible for recognizing evolutionarily conserved pathogen-associated or damage-associated molecular patterns (PAMPs or DAMPs). Once activated, these PRRs invoke innate immune recognition while simultaneously activating the adaptive immune response (Schlee & Hartmann, Nature Reviews Immunology, 16 (9), 566-580 (2016), Paludan, Microbiology and Molecular Biology Reviews, 79 (2), 225-241 (2015)). Binding of PRRs to their corresponding ligand triggers activation of downstream signaling pathways, ultimately resulting in the production of Type I interferons (IFNs) and other proinflammatory cytokines that are essential for initiation of a host of immune functions (Paludan & Bowie, Immunity, 38 (5), 870-880 (2013), Wu & Chen, Annual Review of Immunology, 32 (1), 461-488 (2014)).

Although ligands and binding partners for immunostimulatory receptors are a particularly preferred example of an immunostimulatory agent, and examples are provided in the sections that follow, other adjuvants, including, but not limited to the examples mentioned further below, can also be used.

The immunostimulatory agents can be presented on the nanostructure and/or included as free further agents in formulations with nanostructures, and/or administered as separate formulations to subject in need thereof. In some forms, immunostimulatory agent(s) are fused to other elements such as antigens. In a specific embodiment, an antigen-immunostimulatory molecule is eOD-GT8 fused to a pan HLA DR-binding epitope (PADRE) (“eOD-PADRE”).

iii. Pan T Cell “Helper” Epitope Moieties

In some forms, the immunostimulatory agent includes a T cell “helper” epitope, e.g., helper T cell epitope. T cell helper epitopes are non-antigen specific moieties that have a demonstrated capacity to deliver help for antibody responses in vivo, for example, to be able to provide significant helper T-cell activity. In some forms, the described nanostructures are conjugated with one or more peptides or other moieties, such as linear polypeptide constructs, that are efficient at generating an immune response as large multivalent antigens.

In some forms, the described nanostructures are conjugated with a T cell “helper” epitope that is conjugated to an immunostimulatory agent, such as an antigen, such as a viral antigen. In some forms, a T cell “helper” epitope is indirectly conjugated to a nanostructure, for example, via conjugation to an immunostimulatory agent that is conjugated to a surface of a nanostructure. In other forms, a T cell “helper” epitope is directly conjugated to a nanostructure. In all forms, the conjugated T cell “helper” epitope may be tethered to an immunostimulatory agent or a nanostructure surface via a linker. An exemplary linker is an amino acid linker of from about 1 amino acid to about 10 amino acids, inclusive.

Exemplary T cell “helper” epitope moieties include LS3 peptide, having an amino acid sequence: LRFGIVASRANHALV (SEQ ID NO:5); TpD peptide, having an amino acid sequence: ILMQYIKANSKFIGIPMGLPQSIALSSLMVAQ (SEQ ID NO:6); HIV gp41 peptide, having an amino acid sequence: RIQRGPGRAFVT IGK (SEQ ID NO:7); HA307-219 peptide, having an amino acid sequence: PKYVKQNTLKLAT (SEQ ID NO:8); P30 peptide, having an amino acid sequence: FNNFTVSFWLRVPKVSASHLE (SEQ ID NO:9); HBV core epitope peptide, having an amino acid sequence: T PPAYRPPNAP I L (SEQ ID NO:10); Measles virus F protein 288-302 peptide, having an amino acid sequence: EKKRKRSQRYDPGRV (SEQ ID NO:11); KLH peptide, having an amino acid sequence: PI FFLHHSNTDRLWAI (SEQ ID NO:12); EBV peptide, having an amino acid sequence: LYNLRRGTAL (SEQ ID NO:13); Influenza hemagglutinin peptide, having an amino acid sequence: SAGVYQ I LAIYS T (SEQ ID NO:14); Diphtherial peptide, having an amino acid sequence: IVAQS IALSS (SEQ ID NO:15); Clostridium tetani peptide, having an amino acid sequence: KKQYIKANSKFIGITEL (SEQ ID NO:16); Diphtheria2 peptide, having an amino acid sequence: DSETADNLEKTVAALSILPGHGC (SEQ ID NO:17); Cholera toxin peptide, having an amino acid sequence: ALNIWDRFDVFCTLGATTGYLKGNS (SEQ ID NO:18); and PADRE peptide, having an amino acid sequence: AKFVAAWTLKAAA (SEQ ID NO:19). Any other synthetic or naturally derived peptide that broadly loads on HLA/MHCII complexes and stimulates helper CD4 T cells may be considered a “helper” epitope.

a. Pan DR-Binding Epitope (PADRE) Peptide

In some forms, the immunostimulatory agent is a Pan DR-binding epitope (PADRE) plypeptide. PADRE is a universal synthetic peptide binding (MHC)-II receptors, of which Human Leukocyte Antigen (HLA)-DR receptors, present on specific immune cells. This epitope activates antigen specific CD4+ T-cells, while initiating innate immune response (inflammatory cascade). PADRE was also reported as an alternative to more complex carriers, such as KLH/BSA or OVA, for prophylaxis and therapeutic vaccines to increase vaccines' effectiveness and facilitate large-scale production. PADRE has an affinity for 15 of the 16 most common HLA-DR types. Moreover, its ability to bind HLA-DR receptors, despite their extreme polymorphism in the human population, makes it a tool of choice to study autoreactive T-cell responses. Exemplary PADRE peptides include but are not limited to the biotinylated, the scrambled version, and the restricted version of this PADRE peptide. In some forms, the immunostimulatory agent is a biotinylated PADRE peptide having an amino acid sequence: Biotin-AKFVAAWTLKAAA (SEQ ID NO:19). In some forms, the immunostimulatory agent is a scrambled version of the PADRE peptide having an amino acid sequence: AAATLWKAAKFVA(SEQ ID NO:20). In some forms, the immunostimulatory agent is a scrambled version of the PADRE peptide having an amino acid sequence: ak(Cha)VAAWTLKAAa-Ahx-C(SEQ ID NO:21).

In an exemplary form, the helper T cell epitope, e.g., a PADRE peptide or LS3, is fused directly to an antigen, e.g., an eOD-GT8 antigen (i.e., “eOD-PADRE”, “eOD-LS3”).

iv. Carbohydrate

In some forms, the immunostimulatory agent is or includes a carbohydrate component, such as a monosaccharide, polysaccharide, etc.

As described in more detail in the Examples, it has been established that mono-, oligo- and polysaccharides that are recognized by carbohydrate-binding moieties can be used for selectively stimulating, enhancing or otherwise tuning an immune response in vivo. For example, in some forms, the distribution, surface density and composition of carbohydrate moieties conjugated to a nanostructure, such as N-linked or O-linked glycosylation of glycoprotein antigens, can be designed to illicit a specific immune response in vivo., such as stimulation of the complement system pathway. In some forms, carbohydrates can be introduced through the conjugation of densely glycosylated protein antigens (e.g., eOD-GT8 produced with kifunensine containing 4 N-linked glycosylation sites) or by direct conjugation or hybridization of synthetic glycans onto the nanoparticle surface (e.g., trimannose-azide or brush-like glycan structures). These carbohydrates can be attached with or without the presence of protein antigens or other immunostimulatory molecules. N-linked glycosylation sites or synthetic glycan patches must be densely positioned with a minimum inter-site spacing of 0.5 nm with a maximum distance of 6 nm. Each site or glycosylation site may contain natural or synthetic glycans selected from the group consisting of mono-, oligo-, or polysaccharides.

### (a) Mannose

In some forms, an immunostimulatory agent includes mannose. Mannose is a monosaccharide sugar (C6H1206) that is a C-2 epimer of glucose. In an exemplary form, an immunostimulatory agent includes a mannose homopolymers, such as a β1-4-linked mannose polymer, or a mannose heteropolymers with other sugars moieties, such as glucose, xylose, and galactose. In some forms, an immunostimulatory agent includes mannose oligosaccharides, such as mannan oligosaccharides (MOS). In some forms, an immunostimulatory agent includes a mannose glycoprotein, such as an N-linked oligomannose glycan. In some forms, an immunostimulatory agent includes mannose in the context of O-linked glycosylation. In a particular form, an immunostimulatory agent includes a high-mannose N-glycans that contains multiple mannose residues, for example, terminal mannose that forms a ligand for a mannose receptor (MR; also known as Cluster of Differentiation 206, CD206). MR is a C-type lectin present at the surface of a subset of immune cells (such as macrophages, immature dendritic cells) as well as other cells (such as human dermal fibroblasts and keratinocytes). MR recognizes terminal mannose, N-acetylglucosamine and fucose residues on glycans attached to proteins and functions to stimulate the innate and adaptive immune systems. High mannose structures (e.g., those containing Man9 and/or al-2 or al-3 linkages) additionally recruit mannan binding lectin (MBL, also called mannan-binding lectin or mannan-binding protein (MBP)), which is a oligomeric lectin with carbohydrate recognition domains that binds to hydroxyl domains on branched sugars, primarily on D-mannose, L-fucose, or N-acetylglucosamine.

In some forms, the described nanostructures include a plurality of immunostimulatory agents conjugated to a surface of the nanostructure with a surface density ranging from 1 to 10,000 mannose molecules per nanostructure. The mannose molecules can be in the form of a glycoprotein or glycopeptide. For example, in some forms, the described nanostructures include one or more individual clusters of oligomannose, for example, conjugated to a single antigenic glycopeptide molecule or macromolecule. In some forms, the number of mannose molecules per glycoppetide ranges from 1 to 100, inclusive. In some forms, molecules of mannose are attached to an immunostimulatory agent that is conjugated to the nanostructure, such that each mannose molecule(s) is indirectly conjugated to the nanostructure. In some forms, molecules of mannose are attached directly to a surface of a nanostructure.

In some forms, the specific structure, configuration and orientation of two or more molecules of mannose attached to an immunostimulatory agent, or directly to a nanostructure can be varied, for example, to include various architectures, such as linear or branched oligo-mannose.

### B. Nucleic Acid Nanostructures

The disclosed nucleic acid nanostructures can be formed from any nucleic acid. In preferred embodiments, the nanostructure is formed of DNA. The basic technique for creating nucleic acid nanostructures of various shapes (also referred to herein as nucleic acid or DNA origami) typically involves folding a long single stranded polynucleotide, referred to as a “scaffold strand,” into a desired shape or structure using a number of small “staple strands” as glue to hold the scaffold in place. Several variants of geometries can be used for construction of nucleic acid nanostructures. For example, in some embodiments, nucleic acid nanostructure from purely shorter single stranded staples can be assembled, or nucleic acid nanostructure including purely a single stranded scaffold folded onto itself, any of which can take on diverse geometries/architectures including wireframe or bricklike objects.

The nanostructure is typically in the range of less than 0.5 nm up to 1,000 nm, inclusive or exclusive. In some embodiments, the nanostructure is between 1 nm and 1,000 nm inclusive or exclusive, or range of two integers there between, or any specific integer or fraction of an integer (e.g., to the nearly tenth), there between. The size can be the size of the structure with or without antigen, and/or with or without other elements discussed in more detail elsewhere herein. For example, in some embodiments, the size or range of sizes of the nanostructure is determined before antigen and/or other elements are added thereto (i.e., a naked nanostructure). In other embodiments, the size or range of sizes of the size or range of sizes of the nanostructure is determined after antigen and/or other elements are added thereto.

Nucleic acid nanostructures are nucleic acid assemblies of any arbitrary geometric shapes. Nucleic acid nanostructures can be of two-dimensional shapes, for example plates, or any other 2-D shape of arbitrary sizes and shapes. In some embodiments, the nucleic acid nanostructures are simple DX-tiles, with two DNA duplexes connected by staples. DNA double crossover (DX) motifs are examples of small tiles (˜4 nm×−16 nm) that have been programmed to produce 2D crystals (Winfree, et al., Nature. 394:539-544(1998)); often, these tiles contain pattern-forming features when more than a single tile constitutes the crystallographic repeat. In some embodiments, nucleic acid nanostructures are 2-D crystalline arrays by parallel double helical domains with sticky ends on each connection site (Winfree, et al., Nature. 6;394(6693):539-44 (1998)). In other embodiments, nucleic acid nanostructures are 2-D crystalline arrays by parallel double helical domains, held together by crossovers (Rothemund, et al., PLoS Biol. 2:2041-2053 (2004)). In some embodiments, nucleic acid nanostructures are 2-D crystalline arrays by an origami tile whose helix axes propagate in orthogonal directions (Yan, et al., Science.301:1882-1884 (2003)).

In some embodiments, nucleic acid nanostructures are three-dimensional wireframe nucleic acid assemblies of a uniform polyhedron that has regular polygons as faces and is isogonal. In some embodiments, nucleic acid nanostructures are wireframe nucleic acid assemblies of an irregular polyhedron that has unequal polygons as faces. In some embodiments, nucleic acid nanostructures are wireframe nucleic acid assemblies of a convex polyhedron. In some further embodiments, nucleic acid nanostructures are wireframe nucleic acid assemblies of a concave polyhedron. In some further embodiments, nucleic acid nanostructures are brick-like square or honeycomb lattices of nucleic acid duplexes in cubes, rods, ribbons or other rectilinear geometries. The corrugated ends of these structures are used to form complementary shapes that can self-assemble via non-specific base-stacking.

Some exemplary superstructures of nucleic acid nanostructures include Platonic, Archimedean, Johnson, Catalan, and other polyhedral. In some embodiments, Platonic polyhedron are with multiple faces, for example, 4 face (tetrahedron), 6 faces (cube or hexahedron), 8 faces (octahedron), 10 faces (decahedron), 12 faces (dodecahedron), 20 faces (icosahedron). In some embodiments, nucleic acid nanostructures are toroidal polyhedra and other geometries with holes. In some embodiments, nucleic acid nanostructures are wireframe nucleic acid assemblies of any arbitrary geometric shapes. In some embodiments, nucleic acid nanostructures are wireframe nucleic acid assemblies of non-spherical topologies. Some exemplary topologies include nested cube, nested octahedron, torus, and double torus. In some embodiments, nucleic acid nanostructures are in the form of a bundle or lattice of nucleic acid duplexes (e.g., a 6-helix bundle or a 4-helix bundle or other bundle cross-section with arbitrary number of duplexes on either a square or honeycomb lattice, where the honeycomb lattice may be filled or hollow).

In particular embodiments, the nanostructure of the disclosed compositions can be in the form of a 6-helix bundle. In some embodiments, the nanostructure is shaped as a polyhedron, for example, an icosahedron. In some embodiments, the nanostructure is shaped as a decahedron, such as a pentagonal bipyramid. In some embodiments, the nanostructure is a 6-helix bundle or a polyhedron such as an icosahedron, or a decahedron, such as a pentagonal bipyramid, with HIV antigen, such as eOD-GT6 or eOD-GT8 bound, or another antigen such as p5 or p31, attached thereto.

### 1. Nucleic Acid Scaffold Sequences

Nucleic acids for use in the described nanostructures can be synthetic or natural nucleic acids. In some embodiments, the nucleic acid sequences are not naturally occurring nucleic acid sequences. In some embodiments, the nucleic acid sequences are artificial or otherwise user defined nucleic acid sequences. Nucleic acid sequences that are artificial or otherwise user defined are typically non-naturally occurring nucleic acid sequences and can also be referred to as synthetic nucleic acid sequences.

In some embodiments, the nucleic acid nanostructures are not the genomic nucleic acid of a virus. In some embodiments, the nucleic acid nanostructures are virus-like particles.

Numerous other sources of nucleic acid samples are known or can be developed and any can be used with the described nanostructures, compositions and methods. In some embodiments, nucleic acids used in the described methods are naturally occurring nucleic acids. Examples of suitable nucleic acid samples for use with in the described methods include DNA including genomic DNA samples, RNA samples, cDNA samples, nucleic acid libraries (including cDNA and genomic libraries), whole cell samples, environmental samples, culture samples, tissue samples, bodily fluids, and biopsy samples.

Nucleic acid fragments are segments of larger nucleic molecules. Nucleic acid fragments generally refer to nucleic acid molecules that have been cleaved. A nucleic acid sample that has been incubated with a nucleic acid cleaving reagent is referred to as a digested sample. A nucleic acid sample that has been digested using a restriction enzyme is referred to as a digested sample. In certain embodiments, the nucleic acid sample is a fragment or part of genomic DNA, such as human genomic DNA. Human genomic DNA is available from multiple commercial sources (e.g., Coriell #NA23248). Therefore, nucleic acid samples can be genomic DNA, such as human genomic DNA, or any digested or cleaved sample thereof. Generally, an amount of nucleic acids between 375 bp and 1,000,000 bp is used per nucleic acid nanostructure.

Although only a single nucleic acid strand is typically used as a scaffold sequence for folding the nanostructures, the reverse complement of the nucleic acid strand is used as an alternative for all applications.

M13 is a common source of scaffold strand with native protein-coding sequence and approximately 7k bases. Sequence-controlled scaffold strands may also be produced using helper plasmids (Shepherd, et al., bioRxiv 521443 (2019), doi: https://doi.org/10.1101/521443 and Praetorius et al, Nature, 552:84-87 (2017), Chasteen, et al., Nature, 34(21):e145 (2006)) or using a hybrid synthetic-enzymatic approach (Plesa, et al., Science, 359(6373):343-347 (2018), DOI: 10.1126/science.aao5167), or purely enzymatic approach (Veneziano, et al., Scientific Reports, 8, Article number: 6548 (2018)).

### 2. Staple Strands

The number of staple strands will depend upon the size of the scaffold strand and the complexity of the shape or structure to be formed. For example, for relatively short scaffold strands (e.g., about 50 to 1,500 bases in length) and/or simple structures, the number of staple strands are small (e.g., about 5, 10, 50 or more). For longer scaffold strands (e.g., greater than 1,500 bases) and/or more complex structures, the number of staple strands are several hundred to thousands (e.g., 50, 100, 300, 600, 1,000 or more helper strands).

Typically, staple strands include between 10 and 600 nucleotides, for example, 14-600 nucleotides.

Using a DNA nanostructure for illustration, in scaffolded DNA origami, a long single-stranded DNA can be associated with complementary short single-stranded oligonucleotides that bring two distant sequence-space parts of the long strand together to fold into a defined shape. A robust computational-experimental approach can be used to generate DNA-based wireframe polyhedral structures of arbitrary scaffold sequence, symmetry and size (Jun et al., ACS Nano, 2019, 10,1021/acsnano,8b08671). Staple strands are typically provided in a folding buffer. The staple strands are typically added to the single-stranded scaffold sequence in molar excess, in combination with appropriate salts and detergents. Staple strands may be produced using liquid state synthesis or more commonly solid-state synthesis, or alternatively biologically using phage (Praetorius et al., Nature, 552:84-87 (2017)).

### 3. Purification Tags

In addition to nucleic acid overhangs, other purification tags can be incorporated into the overhang nucleic acid sequence in any nucleic acid nanostructures for purification. In some embodiments, the overhang contains one or more purification tags. In some embodiments, the overhang contains purification tags for affinity purification. In some embodiments, the overhang contains one or more sites for conjugation to a nucleic acid, or non-nucleic acid molecule. For example, the overhang tag can be conjugated to a protein, or non-protein molecule, for example, to enable affinity-binding of the nucleic acid nanostructures. Exemplary proteins for conjugating to overhang tags include biotin and antibodies, or antigen-binding fragments of antibodies. Purification of antibody-tagged nucleic acid nanostructures can be achieved, for example, via interactions with antigens, and or protein A, G, A/G or L.

Further exemplary affinity tags are peptides, nucleic acids, lipids, or mono-, oligo-and polysaccharides. For example, if an overhang contains monosaccharides such as mannose molecules, then mannose-binding lectin can be used for selectively retrieving mannose-containing nucleic acid nanostructures, and vice versa. Other overhang tags allow further interaction with other affinity tags, for example, any specific interaction with magnetic particles allows purification by magnetic interactions.

### 4. Nucleic Acid Overhang Tag

In some embodiments, the overhang sequences are between 4 and 60 nucleotides, depending on user preference and downstream purification techniques. In preferred embodiments, the overhang sequences are between 4 and 25 nucleotides. In some embodiments, the overhang sequences contain 4, 5, 6, 7, 8, 9, 10, 11, 12, 13, 14, 15, 16, 17, 18, 19, 20, 21, 22, 23, 24, 25, 30, 35, 40, 45, 50 nucleotides in length.

In some embodiments, these overhang tag sequences are placed on the 5′ end of any of the staples used to generate a wireframe nucleic acid. In other embodiments, these overhang tag sequences are placed on the 3′ end of any of the staples used to generate a wireframe nucleic acid. Staples or scaffold may also contain sequences that act as barcodes for qPCR-based or sequencing-based determination of cellular or tissue or organ localization and copy number (Okholm, et al., Methods, 67(2):193-7 (2014) doi:10.1016/j.ymeth.2014.01.013, Dahlman, et al., Proc Natl Acad Sci USA, 114(8):2060-65 (2017), doi: 10.1073/pnas.1620874114).

### 5. Additional Functional Elements

i. Agents

In some embodiments, nucleic acid nanostructures are modified by covalent or non-covalent association with a therapeutic agent, a toxic agent, a prophylactic agent, a diagnostic agent, or other agent, particularly protein- or nucleic acid-based therapeutic, functional nucleic acid, prophylactic, or diagnostic agents. For example, one or more therapeutic, prophylactic, or diagnostic agents can be associated with the exterior of the nucleic acid nanoparticle, or packaged within the interior space of the nucleic acid nanoparticle, according to the design of the particle and location of the capture tag or site of interaction with the therapeutic or prophylactic or diagnostic agent.

Exemplary agents that can be used include proteins, peptides, carbohydrates, nucleic acid molecules, polymers, small molecules, and combinations thereof. In some embodiments, the nucleic acid nanoparticles are used for the presentation and/or delivery of a peptide, drug, dye, antibody, or antigen-binding fragment of an antibody.

Therapeutic agents can include anti-cancer, anti-inflammatories, or more specific drugs for inhibition of the disease or disorder to be treated. These may be administered in combination.

Suitable genetic therapeutics include anti-sense DNA and RNA as well as DNA coding for proteins, mRNA, miRNA, piRNA and siRNA. In some embodiments, the nucleic acid that forms the nanoparticles include one or more therapeutic, prophylactic, diagnostic, or toxic agents.

a. Gene Editing Molecules

In certain embodiments, the nucleic acid nanostructures are functionalized to include gene editing moieties, or to include components capable of binding to gene editing moieties.

Exemplary gene-editing moieties that can be included within or bound to nucleic acid nanoparticles are CRISPR RNAs (e.g., single-guide- or CRISPR-RNAs (sg- or crRNA)) for the gene editing through the CRISPR/Cas system, Cas protein or nucleic acids encoding a Cas protein, encoding TAL effector proteins, or zinc-finger proteins, or constructs encoding them, and triplex forming oligonucleotides.

b. mRNA

In some embodiments, nucleic acid nanostructures are modified by covalent or non-covalent association with an RNA that encodes one or more polypeptides, such as a protein. Therefore, in some embodiments, nucleic acid nanostructures are modified to include one or more messenger RNA molecules (mRNA). The messenger RNA can encode any protein or polypeptide. For example, in some embodiments, nucleic acid nanostructures are modified to include one or more mRNAs, each encoding one or more proteins. In an exemplary embodiment, the mRNA encodes a fluorescent protein or fluorophore. Exemplary fluorescent proteins include mCherry, mPlum, mRaspberry, mStrawberry, tdTomato, GFP, EBFP, Azurite, T-Sapphire, Emerald, Topaz, Venus, mOrange, AsRed2, and J-Red. In some embodiments, nucleic acid nanostructures are modified to include one or more messenger RNA molecules an RNA that encodes one or more polypeptides, such as a protein that is an antigen.

In some embodiments, functionalized nucleic acid nanostructures include one or more single-strand overhang or scaffold DNA sequences that are complementary to the loop region of an RNA, such as an mRNA. In one exemplary case, a tetrahedron (but could be any other object that can be designed from the procedure) can be functionalized with 3 (or 1 or 2 or more than 3) single-strand overhang DNA sequences that are complementary to the loop region of an RNA, for example an mRNA, for example an mRNA expressing a protein.

c. Functional Nucleic Acids

In some embodiments, the nucleic acid nanostructures include one or more functional nucleic acids. Functional nucleic acids that inhibit the transcription, translation or function of a target gene are described.

Functional nucleic acids are nucleic acid molecules that have a specific function, such as binding a target molecule or catalyzing a specific reaction. As discussed in more detail below, functional nucleic acid molecules can be divided into the following non-limiting categories: antisense molecules, siRNA, miRNA, aptamers, ribozymes, triplex forming molecules, RNAi, and external guide sequences. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA or the genomic DNA of a target polypeptide or they can interact with the target polypeptide itself. Functional nucleic acids are often designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place. Therefore, the compositions can include one or more functional nucleic acids designed to reduce expression or function of a target protein.

Methods of making and using vectors for in vivo expression of the described functional nucleic acids such as antisense oligonucleotides, siRNA, shRNA, miRNA, EGSs, ribozymes, and aptamers are known in the art.

In some embodiments, the functional nucleic acids, for example siRNA, are designed for targeted knock-down of immune cells based on antigen-recognition and uptake. Exemplary strategies for utilizing siRNA and other therapies in the treatment and prevention of HIV are discussed in Bobbin, et al., Genome Med., 7(1):50 (2015) doi: 10.1186/s13073-015-0174-y, any of which can be used in combination with the disclosed compositions and methods.

ii. Targeting Elements

Targeting elements can be added to the staple strands of the DNA nanostructures, to enhance targeting of the nanostructures to one or more cells, tissues or to mediate specific binding to a protein, lipid, oligo- or polysaccharide, nucleic acid, etc. For example, for use as biosensors, additional nucleotide sequences are included as overhang sequences on the staple strands.

Exemplary targeting elements include proteins, peptides, nucleic acids, lipids, mono-, oligo- and polysaccharides or their synthetic analogs that bind to one or more targets associated with an organ, tissue, cell, or extracellular matrix, or specific type of tumor or infected cell. Typically, the targeting moieties exploit the surface-markers specific to a group of cells to be targeted. The degree of specificity with which the nucleic acid nanostructures are targeted can be modulated through the selection of a targeting molecule with the appropriate affinity and specificity. For example, antibodies, or antigen-binding fragments thereof are very specific.

Additional functional groups can be introduced on the staple strand for example by incorporating biotinylated nucleotides into the staple strand. Any streptavidin-coated targeting molecules are therefore introduced via biotin- streptavidin interaction. In other embodiments, non-naturally occurring nucleotides are included for desired functional groups for further modification. Exemplary functional groups include targeting elements, immunomodulatory elements, chemical groups, biological macromolecules, and combinations thereof.

In some embodiments, nanostructures include one or more sequences of nucleic acids that act as capture tags, or “bait” sequences to specifically bind one or more targeted molecules.

The Examples demonstrate that increasing antigen density and decreasing inter-antigen spacing resulted in an increase in deposition of mannose binding lectin (MBL) and C3 in mouse serum in vitro, and this correlated with improved follicular targeting in vivo. Thus, in some forms, the targeting element is a lectin or a ligand that binds to a lectin, e.g., a glycan, a carbohydrate, polysaccharide, etc. The targeting element can be any lectin or ligand that binds to a target lectin, and preferably induces or enhances lectin-mediated activity, e.g., signaling. In some forms, antigen is opsonized by mannose binding lectin and C3, which promotes complement receptor-mediated interactions between subcapsular sinus macrophages, follicular (non-cognate B cells), and follicular dendritic cells.

Lectins are carbohydrate-binding proteins that selectively recognize and reversibly bind to sugar groups that are part of other molecules. Exemplary carbohydrates to which lectins bind include but are not limited to d-fructose, d-galactose, 1-arabinose, d-xylose, d-mannose, d-glucose, d-glucosamine, d-galactosamine, 1-fucose, various uronic acids, sialic acid, and their combinations to form other di- and oligosaccharides, or other biomolecules. In some forms, the targeting element is or includes dense display of high-mannose glycans.

Lectins are classified based on their structure or subcellular location. Division based on their location includes integral lectins located in membranes as structural components, or soluble lectins present in intra- and intercellular fluids, which can move freely.

Division according to lectin structure consists of several different types of lectins, such as C-type lectins (binding is Ca²⁺ dependent), I-type lectins (carbohydrate recognition domain is similar to immunoglobulins), galectin family (or S-type, which are thiol dependent), pentraxins (pentameric lectins) and β-type lectins (specific to glycoproteins containing mannose 6-phosphate) (Santos, et al., Curr. Top. Pept. Protein Res. 2014; 15:41-62).

Exemplary C-type lectin receptors that can serve as the target include but are not limited to proteoglycans or lecticans such as aggrecan, brevican, neurocan, versican; and Prolectin (C-type lectin domain containing 17A). Exemplary L-type lectins that can serve as the target include but are not limited to Calnexin (binding affinity for Non-reducing glucose residues in an oligosaccharide (Glc(Man)9(GlcNAc)2); Lectin, mannose-binding 1 (affinity for α-(1-2) mannans with free OH-3, OH-4 and OH-6); and Calreticulin (Non-reducing glucose residues in an oligosaccharide (Glc(Man)9(GlcNAc)2).

In some forms, the lectin is a chitolectin or chilectin e.g., chitinases and chitolectins, which recognizes and binds chitins. Chitinases are active proteins that bind and hydrolyze oligosaccharides and chitolectins are able to bind oligosaccharides but do not hydrolyze them (Dalal P., et al., Biochem. Biophys. Res. Commun. 2007; 359:221-226; Kilpatrick et al., Biochim. Biophys. Acta. 2002; 1572:187-197). Exemplary chitinases and chitolectins that can serve as the target include but are not limited to Chitinase 3 like 1 and oviductin or oviductal glycoprotein 1 (recognizes and binds chitin); Chitinase 3 like 2 (recognizes and binds Chitooligosaccharides); and Stabilin-1 interacting chitinase-like protein (recognizes and binds N-acetylgalactosamine (GalNAc), N-Acetylglucosamine (GlcNAc), ribose, and mannose).

Additional lectins that can serve as the target are reviewed in Raposo et al., Biomolecules, 11(2):188 (2021), which is incorporated herein by reference in its entirety.

In one particular form, the lectin activates the lectin pathway or lectin complement pathway. The lectin pathway may be activated in the absence of immune complexes. The lectin pathway is initiated when pattern-recognition molecules (MBL, CL-K1, and ficolins) bind to the so-called pathogen-associated molecular patterns (PAMPs) (D-mannose, N-acetyl-D-glucosamine, or acetyl groups), on the surface of pathogens or to apoptotic or necrotic cells. Circulating MBL, CL-K1, and ficolins form complexes with two dimers of MASPs, MASP-1 and MASP-2. After the binding of MBL/MASPs, CL-K1/MASPs, or ficolin/MASPs complexes to their targets, MASP-1 can auto-activate and trigger MASP-2, leading to C4 and C2 cleavage. This allows the assembly of the C3 and C5 convertases, with subsequent activation of C3 and C5, respectively, and generation of C3a and C5a, two pro-inflammatory anaphylatoxins that increase the inflammatory response. The fragment C3b binds covalently to hydroxyl and amino groups on the surface of target molecules of all three pathways. In the absence of complement regulatory proteins, a powerful amplification in the number of surface-bound C3b molecules takes place through the alternative pathway. In this amplification loop, factor B binds to the attached C3b and is cleaved by factor D generating the alternative pathway C3 convertase C3bBb, which leads to accelerated C3b formation. C3b tags antigens/pathogens for opsonization and antigen presentation or killing by phagocytes through the interaction with complement membrane receptors CR1, CR2, CR3, and CR4, and the immunoglobulin superfamily member CRIg. Finally, the complement cascade culminates with the formation of the multiprotein complex (C5b, C6, C7, C8, and C9n) known as terminal complement complex or MAC, which are inserted as pores of up to 11 nm into the cell membrane inducing loss of membrane integrity and ultimately cell death. The lectin pathway of complement activation is further described in Beltrame, et al., Frontiers in Pediatrics, 2: Article 148 (2014), which is incorporated herein in its entirety.

In some forms, the target receptor is a lectin that is a mannose-binding lectin. Mannose-binding lectin is a central recognition molecule of the lectin pathway, synthesized in liver cells and secreted into bloodstream as high molecular weight multimeric complexes. It is a member of the collectin family of proteins, sharing collagen, and carbohydrate-recognition domains (CRD). MBL is known as a C-type lectin due to the ability to recognize sugar moieties in a Ca²⁺-dependent manner, and is also referred as “defense collagen” because of the important role in the innate immunity and pathogen clearance. Mannose-binding lectin is basically formed by a trimer of identical polypeptide chains, each containing a cysteine-rich N-terminal domain, a collagen-like region, an alpha-helical coiled-coil neck domain, and a C-terminal CRD. The three chains are associated by disulfide bonds and form the structural unit of MBL, which, in turn, polymerize into higher-order MBL oligomers. Single MBL trimers are not fully functional, in contrast to dimers and higher-order oligomers, with tetramers predominating in the circulation. The recognition and binding of MBL to its ligands occurs through the CRD domain, and the oligomeric configuration confers multivalent and high avidity binding to targets.

In other forms, the target lectin is ficolin e.g., Ficolin-1, Ficolin-2, and Ficolin-3. Ficolins are pattern-recognition receptors that are able to associate with MASPs and activate the complement system through the lectin pathway, having an essential role in the immune defense against clinically important pathogens. Besides activating complement, they limit infection by stimulating the secretion of interferon gamma (IFN-γ), IL-17, IL-6, tumor necrosis factor alpha (TNF-α), and nitric oxide (NO) by macrophages.

Thus, it was be appreciated that in some forms these targeting moieties, particularly ligands that bind to or are recognized by lectins, can additionally or alternatively be considered immunostimulatory agents as discussed herein. In some forms, the targeted receptor is a complement receptor. For example, in some forms the target is CR1/2 or FDC or a mannose receptor. Any molecule that targets any one or more of these receptors, such as sugars, or aptamers, antibodies, VHH, scFvs that activate the pathway may be include as a targeting moiety/immunostimulatory agent.

### 6. Modifications to Nucleotides

In some embodiments, the nucleotides of the scaffolded nucleic acid (e.g., nucleic acid nanostructure) and/or additional functional element sequences are modified. In some embodiments, the nucleotides of the encapsulated nucleic acid sequences are modified. In some embodiments, the nucleotides of the DNA staple sequences are modified. In some embodiments, the nucleotides of the DNA tag sequences are modified for further diversification of addresses associated with nucleic acid nanostructures.

Examples of modified nucleotides that may be used include, but are not limited to, diaminopurine, S²T, 5-fluorouracil, 5-bromouracil, 5-chlorouracil, 5-iodouracil, hypoxanthine, xantine, 4- acetylcytosine, 5-(carboxyhydroxylmethyl)uracil, 5-carboxymethylaminomethyl-2- thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7- methylguanine, 5-methylaminomethyluracil, 5-methoxyaminomethyl-2-thiouracil, beta-D- mannosylqueosine, 5′-methoxycarboxymethyluracil, 5-methoxyuracil, 2-methylthio-D46- isopentenyladenine, uracil-5-oxyacetic acid (v), wybutoxosine, pseudouracil, queosine, 2- thiocytosine, 5-methyl-2-thiouracil, 2-thiouracil, 4-thiouracil, 5-methyluracil, uracil-5- oxyacetic acid methylester, uracil-5-oxyacetic acid (v), 5-methyl-2-thiouracil, 3-(3-amino-3- N-2-carboxypropyl) uracil, and (acp3)w, 2,6-diaminopurine.

Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amino-modified groups, such as aminoallyl-dUTP (aa-dUTP) and aminohexhylacrylamide-dCTP (aha-dCTP) to allow covalent attachment of amine reactive moieties, such as N-hydroxy succinimide esters (NHS). Nucleic acid molecules may also contain azido-modified groups, such as N6-(6-azido) hexyl-dATP or alkyne- and DBCO-modified groups, such as C8-alkyne-dCTP and 5-DBCO-PEG₄-dCTP for either copper-assisted or strain-promoted azide-based CLICK chemistry.

Locked nucleic acid (LNA) is a family of conformationally locked nucleotide analogues which, amongst other benefits, imposes truly unprecedented affinity and very high nuclease resistance to DNA and RNA oligonucleotides (Wahlestedt C, et al., Proc. Natl Acad. Sci. USA, 975633-5638 (2000); Braasch, D A, et al., Chem. Biol. 81-7 (2001); Kurreck J, et al., Nucleic Acids Res. 301911-1918 (2002)). In some embodiments, the scaffolded DNAs include synthetic RNA-like high affinity nucleotide analogues, such as locked nucleic acids. In some embodiments, the staple strands are synthetic locked nucleic acids.

Peptide nucleic acid (PNA) is a nucleic acid analog in which the sugar phosphate backbone of natural nucleic acid has been replaced by a synthetic peptide backbone usually formed from N-(2-amino-ethyl)-glycine units, resulting in an achiral and uncharged mimic (Nielsen P E et al., Science 254,1497-1500 (1991)). It is chemically stable and resistant to hydrolytic (enzymatic) cleavage. In some embodiments, the scaffolded DNAs are PNAs. In some embodiments, the staple strands are PNAs.

In some embodiments, a combination of PNAs, DNAs, and/or LNAs is used for the nucleic acids encoding the format of information. In other embodiments, a combination of PNAs, DNAs, and/or LNAs is used for the staple strands, overhang sequences, or any nucleic acid component of the disclosed compositions.

### 7. Terminal Modifications to Staple Strands

In some embodiments, some or all of the staple strands include one or more terminal modifications to the 3′end, 5′ end, or both the 3′ and 5′ ends.

For example, terminal PEG modifications to the 3′end, 5′ end, or both the 3′ and 5′ ends of staples can improve stability against exonucleases and/or passivate the nucleic acid nanostructures, for example against unspecific cellular uptake. In particular examples, dendritic or high-molecular weight PEG modifications provide steric hindrance and thus display improved stabilization and passivation properties (see, e.g., Bujold, et al., Chem. Sci., 5: 2449-2455 (2014)). The terminal modifications can be designed to be cleavable (see, e.g., Govan, et al., Bioconjugate Chem., 22(10): 2136-2142 (2011)).

In some embodiments, staple strands are modified at the 5′ end with phosphate groups to enable ligation of nick positions by for example, but not limited to, T4 ligase to confer stability against exonucleases and simultaneously generate topologically interlocked nucleic acid nanostructures that cannot be unfolded without breakage of covalent bonds, thereby providing physicochemical stability (see, e.g., Conway, et al., Chem. Commun., 49: 1172-1174 (2013)).

In some embodiments, staple strands are modified at the 3′ end, 5′ end, or both the 3′ and 5′ ends with functional groups to permit covalent functionalization with antigens and other agents. In some embodiments, staple strands are modified at the 5′ end with a DBCO or TEG-DBCO group that allows conjugation to azide-modified antigens and other agents by strain-promoted CLICK chemistry at quantitative yields on the assembled nucleic acid nanostructure. Other common bioconjugate techniques are applicable, including, but not limited to, amide chemistry and thiol-maleimide CLICK chemistry.

In some embodiments, staple strands are terminally modified with functional groups to implement in situ templated CLICK chemistry-based functionalization of assembled nucleic acid nanostructures. For example, staple overhangs hybridize to a complementary nucleic acid sequence on an antigen (or other agents) and excess antigen is subsequently purified away. The nucleic acid sequence itself is terminally modified with an azido or alkyne group and gets conjugated to the staple terminus (correspondingly bearing an alkyne or azido group) at the nick position adjacent to the staple overhang via copper-assisted CLICK chemistry. This example combines covalent functionalization strategies with sequence-based addressability of the nucleic acid nanostructure. Analogously, other catalyst-dependent bioconjugate techniques are also applicable.

### 8. Coating Agents for Nucleic Acid Nanostructures

In some embodiments, the nucleic acid nanostructures are coated with agents to convey stabilization against endo- and/or exonucleases, passivation, or a combination thereof. Coating agents can be, for example, naturally occurring or synthetic cationic oligomers or polymers or cooligomers or -polymers containing PEG moieties. In a preferred embodiment, the nucleic acid nanostructures are coated with oligolysine conjugated to a PEG moiety. Preferably, this coating agent includes or consists of 10 lysine units conjugated terminally to a linear PEG moiety, preferably with a molecular weight of approximately 5000 Da (see, e.g., Ponnuswamy, et al., Nat. Commun., 8:15654 (2017)).

Other coating strategies include the use of poly(2-dimethylaminoethyl methacrylate (PDMAEMA) and PEG copolymers thereof in, for example, molecular weight range between 5000 Da to 20000 Da (see, e.g., Kiviaho, et al., Nanoscale, 8:11674-11680) as well as linear polyethyleneimine (PEI), for example, in a molecular weight range between 5000 Da and 10000 Da (see, e.g., Ahmadi, et al., Nanoscale, 10:7494-7504 (2018)). Chitosan represents an example of a naturally occurring cationic polymer and, in a molecular weight range between 4000 Da and 6000 Da with deacetylation of more than 90%, can be used to coat nucleic acid nanostructures (see, e.g., Ahmadi, et al., Nanoscale, 10:7494-7504 (2018)).

In some embodiments, coating agents can be minor groove binders that convey stability against endonucleases as well as passivation. Three distinct classes of minor groove binders that represent suitable coating agents with varying sequence specificity and pharmacological profiles are: bisamidines, polyamides and bisbenzimidazoles. Bisamidines, such as DAPI and others derived therefrom or designed de novo, can provide general protection from endonucleases with relatively low levels of sequence specificity (see, e.g., Tuite, et al., Eur J Biochem, 243(1):482-492 (1997). Polyamides, such as Distamycin and Netropsin and others derived therefrom or designed de novo, display higher levels of sequence specificity. In particular, the sequence specificity and avidity of pyrrole-imidazole polyamides can be rationally designed and cocktails of different polyamides can be used to provide general protection of nucleic acid nanostructures from endonucleases (see, e.g., Kawamoto, et al., Bioorg Med Chem, 26(8):1393-1411 (2018)). In a preferred embodiment, pyrrole-imidazole polyamides demonstrated to be non-toxic are used (see, e.g., Dickinson, et al., Proc Natl Acad Sci USA, 95(22):12890-5 (1998)). Bisbenzimidazoles, such as Hoechst dyes and others derived therefrom or designed de novo, represent an alternative class of minor groove binders that can convey stabilization against endonucleases. In a preferred embodiment, bifunctional minor groove binders enable the passivation of nucleic acid nanostructures via conjugation to a PEG moiety, preferably with a molecular weight of approximately 5000 Da. In another preferred embodiment, bifunctional minor groove binders can be covalently tethered to nucleic acid nanostructures via the installment of alkylating agents to improve long-term stabilization (see, e.g., Oyoshi, et al., J Am Chem Soc, 125(16):4752-4 (2003) and Morita, et al., J Clin Invest, 127(7):2815-2828 (2017)).

### III. Design and Preparation of Nucleic Acid Nanostructures

Systems and methods for the automated, step-wise design of a nucleic acid nanostructure having arbitrary geometries are known in the art.

Scaffolded deoxyribonucleic acid (DNA) origami folds a long single-stranded DNA (ssDNA; “scaffold”) into a user-defined shape by slowly annealing the scaffold in the presence of shorter oligonucleotides (“staples”) containing segments or regions of complementary sequences to the scaffold that bring sequences that are far apart in sequence space to nearby locations in Euclidian space. These interactions and geometries are stabilized by specific Watson-Crick base pairing in the presence of salt that uses immobile Holliday junctions (“crossovers”) to constrain neighboring duplexes physically in space. Crossovers are generally engineered to occur between two parallel DNA duplexes at positions closest or nearest between the two or more helices of the DNA within a 1D, 2D, or 3D structure. Scaffolded DNA origami was initiated by William Shih using a combination of parallel and anti-parallel crossovers (Shih, et al., Nature, 427(6975):618-21 (2004)) and subsequently Paul Rothemund using solely anti-parallel crossovers that has become the most ubiquitous form of scaffolded DNA origami (Rothemund, PW, Nature, 440, 297-302 (2006)), where Rothemund used M13 genomic ssDNA as the scaffold, and the technique has been further modified and generalized by numerous laboratories (Sharma, et al., Science, 323, 112-116 (2009); Dietz, et al., Science, 325, 725-730 (2009); Douglas, et al., Nature, 459, 414-418. (2009); Brown, et al., Nanoscale, 7, 16621-16624 (2015); Marchi, et al., Nano Lett, 14, 5740-5747 (2014)) using M13 or Phage lambda DNA.

Additional, top-down design of scaffolded DNA origami nanostructures have been demonstrated to automatically generate the scaffold routing and complementary ssDNA staple strands to self-assemble under appropriate folding conditions into user-defined geometries of 1D, 2D, or 3D shapes (Veneziano, et al., Science, 352, 1534 (2016); Benson, et al., Nature, 523, 441-444 (2015); Douglas, et al., Nucleic Acids Res, 37, 5001-5006 (2009), Jun, et al., ACS Nano, 2019, 10.1021/acsnano.8b08671), and was the subject of work demonstrating generality of sequence design for scaffold DNA (see, for example, US 20030215914 A1, US 20050147962 A1, WO 2017089567 A1, WO 2017089570 A1, and CN 106119269 A).

One tile-based method allowed for generation of 2D wireframe objects (Yan, et al., Science, 301, 1882-1884 (2003)) that was subsequently implemented experimentally using M13-based scaffolded DNA origami to a include diversity of 2D and closed 3D shapes (Zhang, et al., Nat Nanotechnol., 10(9):779-84 (2015)). This latter scaffolded DNA origami approach was subsequently generalized and fully automated for 3D shapes by Veneziano et al., (Veneziano, et al., Science, 352(6293):1534 (2016)) for DX-based polyhedral origami and by Jun et al, ACS Nano, 2019, 10.102/acsnano.8b08671 for honeycomb and other polyhedral DNA origami.

Non-scaffolded DNA origami is an alternative approach that uses purely short strands of synthetic single-stranded DNA to self-assemble via thermally annealed folding large-scale arrays of structured DNA via a process known as ‘tile-based’ assembly (Yan, et al., Science, 301, 1882-1884 (2003); Winfree, et al., Nature, 394, 539-544 (1998); Ke, et al., Science, 338, 1177-1183 (2012); Ke, et al., Nat Chem, 6, 994-1002 (2014)). In vivo production of top-down designed nanoparticles has long been one goal of the field, with recent promising successes in RNA and DNA (Elbaz, et al., Nat Commun, 7, 11179 (2016); Geary, et al., Science, 345, 799-804 (2014); Nickels, et al., Small, 10, 1765-1769 (2014); Han, et al., Science, 358(6369) (2017)).

Historically, scaffolded DNA origami has largely relied on the natural M13 phage genomic single-stranded DNA as the scaffold (Rothemund, Nature, 440, 297-302 (2006)). This is because it is natively single stranded and easy to produce in the bacteria E. coli, and therefore is available at low cost in large quantities. Efforts to increase production of M13 phage DNA have shown success, obtaining up to 410 mg of ssDNA from 1 liter of E. coli growth (Kick, et al., Nano Lett, 15, 4672-4676 (2015)).

Additional composition and methods for making DNA origami structures are discussed in, for example, Dietz H et al (Dietz H et al., Science, 325, 725-730 (2009)), Liu1 et al (Liu et al., Angew. Chem. Mt. Ed., 50. pp. 264-267 (2011)), Zhao et al (Zhao et al, Nano Lett- 11, pp. 2997-3002 (2011)), Woo et al (Woo et al., Nat. Chem, 3, pp. 620-627 (2011)), Torring et al (Torring et al., Chem. Soc. Rev, 40, pp. 5636-5646 (2011), Shepherd, et al, (Shepherd, et al., bioRxiv 521443 (2019)), doi: https://doi.org/10.1101/521443) and Praetorius et al (Praetorius et al, Nature, 552:84-87 (2017)).

Typically, creating a nucleic acid nanostructure includes one or more of the steps of (a) designing the nanostructure; (b) assembling, and optionally labeling, the nanostructure; (c) purifying the assembled nanostructure; (d) conjugating antigen to the nanostructure; and (e) determining or verifying the structure of the nanostructure, each of which is discussed in more detail below.

### A. Design of Nucleic Acid Nanostructures

The nucleic acid nanostructure has a defined shape and size. Typically, one or more dimensions of the nanostructure are determined by the target sequence. The methods include designing nanostructures including the target nucleic acid sequence.

The starting point for the design process can be the selection of a target or desired shape. An exemplary method for designing a nucleic acid nanostructure having a desired polyhedral form includes selecting a desired 3D polyhedral or 2D polygon form as a target structure; providing geometric parameters and physical dimensions of the a target structure for a selected 3D polyhedral or 2D polygon form; identifying the route of a single-stranded nucleic acid scaffold that traces throughout the entire target structure; and generating the sequences of the single-stranded nucleic acid scaffold and/or the nucleic acid sequence of staple strands that combine to form a nucleic acid nanostructure having the desired shape. DNA nanostructures having the desired shape are produced by folding a long single stranded polynucleotide, referred to as a “scaffold strand”, into a desired shape or structure using a number of small “staple strands” as glue to hold the scaffold in place.

Any arbitrary geometric shape that can be rendered as a “wireframe” model can be selected as input for the design of nucleic acid assemblies. Target structures can be selected based upon one or more design criteria, or can be selected randomly. In some embodiments, structures are selected based on existing ‘natural’ 3-dimensional organizations (e.g., virus capsids, antigens, toxins, etc.). Therefore, in some embodiments, target shapes are designed for use directly or as part of a system to mediate one or more biological or other responses which are dependent upon, or otherwise influenced by 3D geometric spatial properties. For example, in some embodiments, all or part of a nanostructure is designed to include architectural features known to elicit or control one or more biological functions. In some embodiments, structures are designed to fulfill the 3D geometric spatial requirements to induce, prevent, stimulate, activate, reduce or otherwise control one or more biological functions. Typically, the desired shape defines a specific geometric form that will constrain the other physical parameters, such as the absolute size of the particle. For example, the minimum size of nucleic acid nanostructures designed according to the described methods will depend upon the degree of complexity of the desired shape.

Nucleic acid nanostructures can be geometrically simple, or geometrically complex, such as polyhedral three-dimensional structures of arbitrary geometry. Target structures can be any solid in two dimensions. Therefore, target structures can be a grid or mesh or wireframe topologically similar to a 2D surface or plane. The grid or mesh can be composed of regular or irregular geometries that can be tessellated over a surface. Exemplary target structures include triangular lattices, square lattices, pentagonal lattices, or lattices of more than 5 sides. 2D structures can be designed to have varied length and thickness in each dimension. In some embodiments, the edges of 2D nanostructures include a single nucleic acid helix. In other embodiments, the edges of 2D nanostructures include two or more nucleic acid helices. For example, in some embodiments, each edge of the 2D nanostructure includes 2 helices, 4 helices, 6 helices, 7 or 8 helices, or more than 8 helices on cubic or honeycomb lattices, up to 100 helices per edge, although theoretically unlimited in number.

Target structures can also be any solid in three dimensions that can be rendered with flat polygonal faces, straight edges and sharp corners or vertices. Exemplary basic target structures include cuboidal structures, icosahedral structures, tetrahedral structures, cuboctahedral structures, octahedral structures, decahedral, and hexahedral structures. In some embodiments, the target structure is a convex polyhedron, or a concave polyhedron. For example, in some embodiments, a nucleic acid nanostructure is shaped as a uniform polyhedron that has regular polygons as faces and is isogonal. In other embodiments, a nucleic acid nanostructure is shaped as an irregular polyhedron that has unequal polygons as faces. In further embodiments, the target structure is a truncated polyhedral structure, such as truncated cuboctahedron.

In some embodiments, the target structure is a nucleic acid assembly that has a non-spherical geometry. Therefore, in some embodiments, the target structure has a geometry with “holes”. Exemplary non-spherical geometries include toroidal polyhedra and nested shapes. Exemplary toroidal polyhedra include a torus and double torus. Exemplary topologies of nested shapes include nested cube and nested octahedron. In other embodiments, target structures can be a combination of one or more of the same or different polyhedral forms, linked by a common contiguous edge.

Any method for the manipulation, assortment or shaping of nucleic acids can be used to produce the nanostructures. Typically, the methods include methods for “shaping” or otherwise changing the conformation of nucleic acid, such as methods for DNA origami.

In some embodiments, nucleic acid nanostructures are designed using methods that determine the single-stranded oligonucleotide staple sequences that can be combined with the target sequence to form a complete three-dimensional nucleic acid nanostructure of a desired form and size. Therefore, in some embodiments, the methods include the automated custom design of nucleic acid nanostructure corresponding to a target nucleic acid sequence. For example, in some embodiments, a robust computational approach is used to generate DNA-based wireframe polyhedral structures of arbitrary scaffold sequence, symmetry and size. In particular embodiments, design of a nanostructure corresponding to the target nucleic acid sequence, includes providing information as geometric parameters corresponding to the desired form and dimensions of the nanostructure, which are used to generate the sequences of oligonucleotide “staples” that can hybridize to the target nucleic acid “scaffold” sequence to form the desired shape. Typically, the target nucleic acid is routed throughout the Eulerian circuit of the network defined by the wire-frame geometry of the nanostructure.

A step-wise, top-down approach has been proven for generating DNA nanostructure origami objects of any regular or irregular wireframe polyhedron, with edges composed of a multiple of two number of helices (i.e., 2, 4, 6, etc.) and with edge lengths a multiple of 10.5 rounded down to the closest integer. Exemplary methods for the top-down design of nucleic acid nanostructures of arbitrary geometry are described in Venziano et al, Science, 352:6293 (2016), Jun et al., ACS Nano, 2019, 10.1021/acsnano.8b08671, WO2017089567A1, and WO2017089570A1, the contents of which are incorporated by reference in their entireties.

In other embodiments, the sequence of the nanostructure is designed manually, or using alternative computational sequence design procedures. Exemplary design strategies that can be incorporated into the methods for making and using NMOs include single-stranded tile-based DNA origami (Ke, et al., Science 2012); brick-like DNA origami, for example, including a single-stranded scaffold with helper strands (Rothemund, et al., and Douglas, et al.); and purely single-stranded DNA that folds onto itself in PX-origami, for example, using paranemic crossovers.

Alternative structured NMOs include bricks, bricks with holes or cavities, assembled using DNA duplexes packed on square or honeycomb lattices (Douglas et al., Nature 459, 414-418 (2009); Ke Y et al., Science 338: 1177 (2012)). Paranemic-crossover (PX)-origami in which the nanostructure is formed by folding a single long scaffold strand onto itself can alternatively be used, provided bait sequences are still included in a site-specific manner. Further diversity can be introduced such as using different edge types, including 6-, 8-, 10, or 12-helix bundle. Further topology such as ring structure is also useable for example a 6-helix bundle ring.

### B. Assembly of Nucleic Acid Nanostructures

The single-stranded nucleic acid scaffold and the corresponding staple sequences are assembled into a nanostructure having the desired shape and size. In some embodiments, assembly is carried out by hybridization of the staples to the scaffold sequence. In other embodiments, nanostructures include only single-stranded DNA oligos. In further embodiments, the nanostructures include a single-stranded DNA molecule folded onto itself. Therefore, in some embodiments, the NMOs are assembled by DNA origami annealing reactions.

Typically, annealing can be carried out according to the specific parameters of the staple and/or scaffold sequences. For example, the oligonucleotide staples are mixed in the appropriate quantities in an appropriate reaction volume. In preferred embodiments, the staple strand mixes are added in an amount effective to maximize the yield and correct assembly of the nanostructure. For example, in some embodiments, the staple strand mixes are added in molar excess of the scaffold strand. In an exemplary embodiment, the staple strand mixes are added at a 10-20× molar excess of the scaffold strand. In some embodiments, the synthesized oligonucleotides staples with and without tag overhangs are mixed with the scaffold strand and annealed by slowly lowering the temperature (annealing) over the course of 1 to 48 hours. In some embodiments, in particular at high numbers of tag overhangs per nucleic acid nanostructure, tag overhangs can be masked with guard strands to avoid aggregation and inter-nanostructure insertion of tag overhangs. This process allows the staple strands to guide the folding of the scaffold into the final nanostructure.

Material usage for assembly can be minimized and assembly hastened by use of microfluidic automated assembly devices. For example, in certain embodiments, the oligonucleotide staples are added in one inlet, the scaffold can be added in a second inlet, with the solution being mixed using methods known in the art, and the mix traveling through an annealing chamber, wherein the temperature steadily decreases over time or distance. The output port then contains the assembled nanostructure for further purification or storage. Similar strategies can be used based on digital droplet-based microfluidics on surfaces to mix and anneal solutions, and applied to purely single-stranded oligo-based nanostructures or single-stranded scaffold origami in the absence of helper strands. Overhang sequences for hybridization of antigen/functional element-oligo conjugates or for covalent attachment of antigen/functional element-click/other chemical groups may be incorporated either using solid-state or liquid-state synthesized oligos, or using bacterially produced oligos for large-scale, low-cost production (Praetorius et al., Nature, 552:84-87 (2017)).

### C. Purification of Nucleic Acid Nanostructures

The methods include purification of the assembled nanostructures. Purification separates assembled structures from the substrates and buffers required during the assembly process. Typically, purification is carried out according to the physical characteristics of nanostructures, for example, the use of filters and/or chromatographic processes (FPLC, etc.) is carried out according to the size and shape of the nanostructures.

In an exemplary embodiment, nanostructures are purified using filtration, such as by centrifugal filtration, or gravity filtration, or by diffusion such as through dialysis. In some embodiments, filtration is carried out using, e.g., an Amicon Ultra-0.5 ml (or larger scales) centrifugal filter (MWCO 100 kDa). In some embodiments, for example, those for which antigen functionalization is not compatible with Amicon filters, Pluronic F-127-passivated Spin-X UF-0.5 ml (or larger scales) centrifugal filters can be used (MWCO 100 kDa).

In some embodiments, nanostructures are purified after assembly, after antigen conjugation, or both.

D. Pairing with Immunostimulatory Agents

Immunostimulatory agents, such as antigens, adjuvants, and other agents can be covalently or non-covalently bound to, or otherwise associated with the nanostructure. The association can before, during or after nanostructure assembly.

In some embodiments, the immunostimulatory agent is directly or indirectly bound to the nanostructure via outwardly facing nucleic acid overhangs extending from the 3′ and/or 5′ ends of selected staple strands. The nucleic acid overhangs can include one or more sequences that is complementary to a target RNA, DNA or PNA sequence covalently linked to the immunostimulatory agent. Such covalent linkage may be formed by, for example, maleimide-thiol coupling. Additionally, or alternatively, the nucleic acid overhangs can include one or more sequences that is complementary to part of all of target RNA, DNA or PNA sequence that is or includes the immunostimulatory agent, e.g., a nucleic acid adjuvant such as a TLR9 ligand. In some embodiments, the immunostimulatory agent is a single stranded nucleic acid that is complementary in part or in total to a nucleic acid overhang of the nanostructure, or a double stranded nucleic acid with a single stranded segment, optionally an overhang, that is complementary in part or in total to a nucleic acid overhang of the nanostructure. When the immunostimulatory agent-nucleic acid is mixed together with nucleic acid overhangs having a complementary sequence, the nucleic acid sequence of the immunostimulatory agent-nucleic acid conjugate can non-covalently hybridize with the nucleic acid overhang.

Other interactions for attachment or incorporation of immunostimulatory agent include, but are not limited to, intercalation, minor groove interactions, biotin- streptavidin interaction, and chemical linkers (e.g., using Click-chemistry groups).

In a particular embodiment illustrated below, an immunostimulatory agent is conjugated to peptide nucleic acid oligomers, which then hybridize with stable strand overhangs on the nucleic acid nanostructure. For example, PNA peptide containing a maleimide formed by, for example, solid phase synthesis, can be dissolved in an aqueous solution and reacted with N-terminal cysteine of an immunostimulatory agent, such as a peptide antigen. Nucleic acid nanostructures can be mixed with the PNA-immunostimulatory agent conjugates, typically molar excess of PNA-immunostimulatory agent conjugates, which hybridize with overhangs on nucleic acid nanostructures.

Alternatively, dCas9 with guide sequence may be used to target specific nucleic acid sequences in the origami particle, or DNA-binding peptides or proteins such as bZIPs or deactivated TALENs or nanobodies that may be sequence-specific or not, or other orthogonal protein decoration of DNA origami (Sacca, et al., Angew Chem Int Ed Engl., 49(49):9378-83 (2010). Doi: 10.1002/anie.201005931), minor groove binders, or polycationic oligomers and polymers that non-covalently condense onto the nucleic acid nanostructure. In all cases, the nucleic acid binding component may constitute the antigen, or template the antigen through covalent or non-covalent interactions.

In some embodiments, the immunostimulatory agent is linked to the nanostructure indirectly and further includes a linker that alters the rigidity or flexibility of the antigen relative to the nanostructure, as above.

Suitable linkers include, but are not limited to, oligonucleotides (e.g., a string of nucleic acids such as single or double stranded DNA, RNA, PNA, or others including those described elsewhere herein), polymers including but not limited to poly(ethylene glycol), polyacrylamide, polyacrylic acid; a string of amino acids (i.e., peptide linker), dextran, or combinations thereof, as listed above.

In some embodiments, the linker is or includes engineered nucleic acid (e.g., DNA) -binding proteins such as those listed above, including inactive endo- or exonucleases, that bind the DNA duplex in specific orientations and positions may be engineered to bear antigenic domains that are subsequently rigidly presented by the nanostructure due to its linking domain.

Linkers can be of any suitable length. Typically, the length is suitable for antigen to induce an immune response. Lengths in the range of 0.6 nm to 52 nm were tested below (see, e.g., Table 3). In some embodiments, the length of the linker is between 0.5 nm and 500 nm, 0.5 nm and 250 nm, 0.5 and 100 nm, 0.5 nm and 75 nm, 0.6 nm and 52 nm, inclusive, or an specific integer length or range of lengths there between, or a specific length exemplified in Table 3. In some embodiments, the linker itself does or does contribute to or otherwise induce an immune response.

The Examples below show that rigid presentation of immunostimulatory agents, such as antigens, is preferred over flexible presentation, whereas flexibility of immunostimulatory agents, such as TLR agonists, is preferred over rigid presentation. Thus, in some embodiments, there is no linker, or only a minimal linker, to facilitate attachment of the antigen to the nanostructure, while encouraging rigid presentation of the antigen on the surface of the structure. In some embodiments, the linker is a rigid linker. In other embodiments, flexibility is preferred. In some embodiments, the linker is a flexible linker.

In some forms, a linker is or includes one or more C-series linkers, such as a C6 linker, a C12, linker, etc. In some forms, a linker is an amino acid linker of 1 to 10 residues. For example, in some forms, a T cell epitope includes a linker of from about 1 amino acid to about 10 amino acids between the T cell epitope and the immunostimulatory agent, such as an antigen. In some forms, the size and/or composition of a linker can impact the function of a T cell epitope. In some forms, a linker is or includes PEG linkers (PEG3), PEG6, etc. In some forms, a linker incorporates different types of linkers including C-series linkers, amino acid linkers, PEG linkers into one linker.

### E. Validation of Nucleic Acid Nanostructures

Methods for designing nucleic acid nanostructures of a desired shape and size can include steps for validation of the resulting nucleic acid structure based on the output sequences. For example, in some embodiments, the methods also include the step of predicting the 3-dimensional coordinates of the nucleic acids within the nucleic acid nanostructure, based on the output of the system used for positioning scaffold and staple sequences. When structural information for a nucleic acid nanostructure is predicted, the predicted information can be used to validate the nucleic acid nanostructure. Typically, validation of the resulting nucleic acid structure includes (1) calculating the positions of each base pair in the structural model; (2) determining the positions of each base pair in the nucleic acid nanostructure; and (3) comparing the calculated structural data obtained for the model with that experimentally determined (i.e., observed) for the nanostructure.

Validation can also include, for example, electron microscopic observation of the structures formed.

### IV. Formulations

### A. Pharmaceutical Compositions

Pharmaceutical compositions containing nucleic acid nanostructures and one or more immunostimulatory agents in combination with a pharmaceutically acceptable carrier, diluent, preservative, excipient, or combination thereof are provided. In some embodiments, the pharmaceutical compositions also contain one or more agents or moieties, such as targeting agent(s) or coating agent(s) for example. Pharmaceutical compositions disclosed herein include any combination and sub-combination of the aforementioned compositions including nucleic acid nanostructures, immunostimulatory agents, and optionally moieties (e.g., targeting agents, coating agent(s), etc.), gene editing molecules, siRNAs, and linear or circular mRNAs, which may also form the scaffold sequence of the nanostructure, in combination with a pharmaceutically acceptable carrier, diluent, preservative, excipient, or combination thereof.

The pharmaceutical compositions disclosed herein can be used in immunogenic compositions and as vaccines or components of vaccines. Typically, immunogenic compositions disclosed herein include a nucleic acid nanostructure, and immunostimulatory agents, such as an antigen, an adjuvant, or a combination thereof. Immunogenic compositions may also include a coating agent. When administered to a subject in combination, the immunostimulatory agents (e.g., adjuvant and/or antigen) can be administered in separate pharmaceutical compositions, or they can be administered together in the same pharmaceutical composition. When present in the same pharmaceutical composition, or administered in combination, an adjuvant and an antigen can be referred to as a vaccine.

Pharmaceutical compositions can be for administration by parenteral (intramuscular, intraperitoneal, intravenous (IV), intradermal, or subcutaneous injection), or transmucosal (nasal, vaginal, rectal, or sublingual) routes of administration or using bioerodible inserts and can be formulated in dosage forms appropriate for each route of administration.

In some embodiments, the compositions are administered systemically, for example, by intravenous or intraperitoneal administration, in an amount effective for delivery of the compositions to targeted cells. In certain embodiments, the compositions are administered locally, for example by injection directly into a site to be treated. Typically, local injection causes an increased localized concentration of the compositions which is greater than that which can be achieved by systemic administration.

In some embodiments, the pharmaceutical compositions are injected or otherwise administered directly to one or more tumors. Most typically, the pharmaceutical compositions are administered by intramuscular, intradermal, subcutaneous, or intravenous injection or infusion, or by intranasal delivery.

In some embodiments, the pharmaceutical compositions are delivered by catheter or syringe. Other means of delivering such compositions include using infusion pumps (for example, from Alza Corporation, Palo Alto, Calif.) or incorporating the pharmaceutical compositions into polymeric implants (see, for example, P. Johnson and J. G. Lloyd-Jones, eds., Drug Delivery Systems (Chichester, England: Ellis Horwood Ltd., 1987), which can affect a sustained release of the composition to the immediate area of the implant.

As further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired effect, on the route of administration, and on the duration of the treatment desired.

An exemplary dosage range for immunostimulatory agents, such as antigen and/or adjuvant components of a vaccine are about 10 μg to about 500 μg of antigen, and about 10 μg to about 1000 μg of adjuvant for in vivo application. In some embodiments, the dosage range of the immunostimulatory agent, such as an antigen is between about 10 ng and about 500 μg, or about 10 ng 100 μg.

Immunostimulatory agents dosages, such as adjuvant dosages, can also be determined based on activity or units. For example, in some embodiments, the unit dosage of an adjuvant is between about 1U and about 10U, or between about 2U and about 7U, or between about 2.5U and about 5U.

### 1. Formulations for Parenteral Administration

In a preferred embodiment, the pharmaceutical compositions are administered in an aqueous solution, by parenteral injection. The formulation can be in the form of a suspension or emulsion. In general, pharmaceutical compositions are provided including an effective amount of the nanostructure and immunostimulatory agent and optionally including additional moieties (e.g., targeting agent, coating agent(s), etc.), pharmaceutically acceptable diluents, preservatives, solubilizers, emulsifiers, adjuvants, and/or carriers. Such compositions can include diluents sterile water, buffered saline of various buffer content (e.g., Tris-HCl, acetate, phosphate), pH and ionic strength; and optionally, additives such as detergents and solubilizing agents (e.g., TWEEN®20, TWEEN®80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate.

The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### 2. Formulations for Topical and Mucosal Administration

The disclosed pharmaceutical compositions can be applied topically. Topical administration can include application to the lungs (pulmonary), nasal, oral (sublingual, buccal), vaginal, or rectal mucosa.

Compositions can be delivered to the lungs while inhaling and traverse across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent® nebulizer (Mallinckrodt Inc., St. Louis, Mo.); the Acorn® II nebulizer (Marquest Medical Products, Englewood, Colo.); the Ventolin® metered dose inhaler (Glaxo Inc., Research Triangle Park, N.C.); and the Spinhaler® powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator.

### 3. Delivery Vehicles

Any of the disclosure compositions can be formulated in a delivery vehicle. Exemplary delivery vehicles include those that enhance delivery of the nanostructures across cell membrane such as the plasma membrane. Exemplary delivery vehicles include, but are not limited to, lipid-based delivery vehicles such as liposome or micelle. Liposomes are spherical vesicles composed of concentric phospholipid bilayers separated by aqueous compartments. Liposomes can adhere to and form a molecular film on cellular surfaces. Structurally, liposomes are lipid vesicles composed of concentric phospholipid bilayers which enclose an aqueous interior (Gregoriadis, et al., Int. J. Pharm., 300, 125-30 2005; Gregoriadis and Ryman, Biochem. J., 124, 58P (1971)). Hydrophobic compounds associate with the lipid phase, while hydrophilic compounds associate with the aqueous phase.

Liposomes and micelles can be formed from one or more lipids, which can be neutral, anionic, or cationic at physiologic pH. Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including, but limited to, 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids. In some embodiments, the liposomes contain a phosphaditylcholine (PC) head group, and optionally sphingomyelin. In some embodiments, the liposomes contain DPPC. In a further embodiment, the liposomes contain a neutral lipid, such as 1,2-dioleoylphosphatidylcholine (DOPC).

In certain embodiments, the liposomes are generated from a single type of phospholipid. In some embodiments, the phospholipid has a phosphaditylcholine head group, and, can be, for example, sphingomyelin. The liposomes may include a sphingomyelin metabolite. Sphingomyelin metabolites used to formulate the liposomes include, without limitation, ceramide, sphingosine, or sphingosine 1-phosphate. The concentration of the sphingomyelin metabolites included in the lipids used to formulate the liposomes can range from about 0.1 mol % to about 10 mol %, or from about 2.0 mol % to about 5.0 mol %, or can be in a concentration of about 1.0 mol %.

Suitable cationic lipids in the liposomes include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1 ,2-dialkyloxy-3-dimethylammonium propanes, dioctadecylamidoglycylspermine (DOGS), 3 -[N-(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N, N, N′, N′-tetramethyl- , N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

The lipids may be formed from a combination of more than one lipid, for example, a charged lipid may be combined with a lipid that is non-ionic or uncharged at physiological pH. Non-ionic lipids include, but are not limited to, cholesterol and DOPE (1,2-dioleolylglyceryl phosphatidylethanolamine). The molar ratio of a first phospholipid, such as sphingomyelin, to second lipid can range from about 5:1 to about 1:1 or 3:1 to about 1:1, or from about 1.5:1 to about 1:1, or the molar ratio is about 1:1.

In some embodiments, liposomes or micelles include phospholipids, cholesterols and nitrogen-containing lipids. Examples include phospholipids, including natural phospholipids such as phosphatidylcholine, phosphatidylserine, phosphatidylglycerol, phosphatidylinositol, phosphatidylethanolamine, phosphatidic acid, cardiolipin, sphingomyelin, egg yolk lecithin, soybean lecithin, and lysolecithin, as well as hydrogenated products thereof obtained in a standard manner. It is also possible to use synthetic phospholipids such as dicetyl phosphate, distearoylphosphatidylcholine, dipalmitoylphosphatidylcholine, dipalmitoylphosphatidylethanolamine, dipalmitoylphosphatidylserine, eleostearoylphosphatidylcholine, eleostearoylphosphatidylethanolamine as well as homo-poly{N′-[N-(2-aminoethyl)-2-aminoethyl]aspartamide}P[Asp(DET)]and block-catiomer poly(ethyleneglycol) (PEG)-b-P[Asp(DET)].

In some embodiments, the liposomes are long circulating liposomes or stealth liposomes such as those reviewed in Immordino, et al, Int J Nano medicine, 1(3):297-315 (2006)), which is specifically incorporated by reference herein in its entirety. For example, liposomes have been developed with surfaces modified with a variety of molecules including glycolipids and sialic acid. Long-circulating liposomes can include, for example, synthetic polymer poly-(ethylene glycol) (PEG) in liposome composition. The PEG on the surface of the liposomal carrier can extend blood-circulation time while reducing mononuclear phagocyte system uptake (stealth liposomes) and serve as an anchor for the targeting moiety.

Antibodies and antibody fragments are widely employed for targeting moieties for liposomes due to the high specificity for their target antigens. Referred to immunoliposomes, methods of generated targeted liposomes by coupling of antibodies to the liposomal surface are known in the art. Such techniques include, but are not limited to, conventional coupling and maleimide based techniques. See also, Paszko and Senge, Curr Med Chem., 19(31):5239-77 (2012), Kelly, et al., Journal of Drug Delivery, Volume 2011 (2011), Article ID 727241, 11 pages.

The micelles can be polymer micelles, for example, those composed of amphiphilic di-or tri-block copolymers made of solvophilic and solvophobic blocks (see, e.g., Croy and Kwon, Curr Pharm Des., 12(36):4669-84 (2006)).

### B. Immunogenic Compositions

The nanostructures disclosed herein can be used in immunogenic compositions or as components in vaccines. Typically, an immunogenic composition includes one or more immunostimulatory agent(s), such as adjuvant, an antigen, or a combination thereof. The combination of an adjuvant and an antigen can be referred to as a vaccine. When administered to a subject in combination, the adjuvant and antigen can be administered in separate pharmaceutical compositions, or they can be administered together in the same pharmaceutical composition. As described herein, the nanostructure can be engineered to present or otherwise contain antigen, and thus the antigen-nanostructure composition can serve as the antigen component of an immunogenic composition or vaccine formulation. Additionally, or alternatively, the nanostructure can include an adjuvant. Thus, in some embodiments, the nanostructure includes both an antigen and an adjuvant. Two or more different nanostructures can be utilized each presenting one or more different antigens, one or more different adjuvants, or combinations thereof.

Specific DNA sequences can be included as adjuvants, with the 3D patterning in geometry and size controlled in an arbitrary manner scaffolded by the nucleic acid nanostructure.

In some embodiments, the nanostructure does not include an adjuvant. In some embodiments, the nanostructure includes an adjuvant that is used alone or in combination with an antigen that may or may not be linked to a nanostructure. Thus, in some embodiments, an adjuvant can be administered together (e.g., in the same pharmaceutical composition) or separately (e.g., in a different pharmaceutical composition) from a nanostructure containing the antigen. In some embodiments, an antigen can be administered together (e.g., in the same pharmaceutical composition) or separately (e.g., in a different pharmaceutical composition) from a nanostructure containing an adjuvant.

### C. Adjuvants

Immunostimulatory agents include immunologic adjuvants. Adjuvants stimulate the immune system's response to a target antigen, but do not provide immunity themselves. Adjuvants can act in various ways in presenting an antigen to the immune system. Adjuvants can act as a depot for the antigen, presenting the antigen over a longer period of time, thus maximizing the immune response before the body clears the antigen. Examples of depot type adjuvants are oil emulsions. An adjuvant can also act as an irritant, which engages and amplifies the body's immune response.

The adjuvant may be without limitation alum (e.g., aluminum hydroxide, aluminum phosphate); saponins purified from the bark of the Q. saponaria tree such as Quil A (a mixture of more than 25 different saponin molecules), or subcombinations or individual molecules thereof such as QS21 (a glycolipid that elutes in the 21^{st} peak with HPLC fractionation; Antigenics, Inc., Worcester, Mass.); poly [di(carboxylatophenoxy)phosphazene (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS 1312, water-based nanoparticles combined with a soluble immunostimulant, Seppic). In other forms, the adjuvant includes an oil-in-water-based emulsion, for example, such as MF59. Exemplary oil-in-water based emulsions are described in U.S. Pat. No. 8,778,275 B2, the content of which is incorporated by reference herein in its entirety.

Adjuvants may be TLR ligands, such as those discussed above. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland). Adjuvants that act through TLR5 include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

The adjuvant can also be anoil emulsion (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-.gamma.), macrophage colony stimulating factor, and tumor necrosis factor.

Immunostimulatory complexes called ISCOMs are particulate antigen delivery systems having antigen, cholesterol, phospholipid and saponin (Quil A or other saponin) with potent immunostimulatory activity. ISCOMATRIX® is a particulate adjuvant having cholesterol, phospholipids and saponins (Quil A) but without containing antigen. See, e.g., U.S. Pat. No. 9,149,520, Sun, et al., Volume 27, Issue 33, 16 Jul. 2009, Pages 4388-4401, and Morelli, et al., J Med Microbiol. 2012 July;61(Pt 7):935-43. Doi: 10.1099/jmm.0.040857-0. Epub 2012 Mar 22. This adjuvant has principally the same structure as ISCOMs, consisting of perforated cage-like particles of approximately 40 nm in diameter. The antigens can be formulated with ISCOMATRIX® to produce vaccines capable of antigen presentation and immunostimulants similar to ISCOMs-type formulations, but with a wider range of applicability, since its use is not limited to hydrophobic membrane proteins. Modifications of ISCOMs formulations and ISCOMATRIX® have also been developed to achieve a better association of some antigens, such as described in WO 98/36772.

ISCOMs and ISCOMATRIX® combine the advantages of a particulate delivery system with the in situ presence of an adjuvant (Quil A) and consequently have been found to be more immunogenic than other colloidal systems such as liposomes and protein micelles. Formulations of ISCOMs and ISCOMATRIX® retained the adjuvant activity of the Quil A, while increasing its stability, reducing its hemolytic activity, and producing less toxicity. They also generate a similar immune response to the one obtained by immunizing with simple mixtures of antigen and saponin, but allow for the use of substantially smaller amounts of antigen. Several ISCOMs-type vaccine formulations or containing ISCOMATRIX® have been approved for veterinary use, for example against equine influenza virus.

Other liposomal systems mainly composed of saponins from Q. saponaria and sterols (primarily cholesterol) have been described, one of which is referred to as ASO1B. See, e.g., WO 96/33739, being also formulated as emulsions such as described in US 2005/0220814. See, also, U.S. Published Application No. 2011/0206758. Adjuvants may also include saponin MPLA nanoparticles (SMNP), for example, as described in Silva, et al., Science Immunology, V.6 (66) (2021), the content of which is incorporated by reference herein in its entirety.

Iscomatrix-like adjuvants such as ISCOMATRIX® are thought to function via canonical inflammasome activation and subsequent release of pro-inflammatory cytokines such as IL-18 and IL-10 (Wilson, et al., Journal of immunology. 2014; 192(7):3259-68. Doi: 10.4049/jimmunol.1302011. PubMed PMID: 24610009). This mechanism is thought to be mediated at least in-part by endosomal degradation and the release of NRLP3-activating cathepsin proteases into the cytosol.

### V. Methods

Disclosed are various methods of using the provided compositions. For example, the compositions may be administered as an immunostimulatory agent. In some embodiments, the nanostructure is administered as part of a vaccine and/or as part of a method of treatment. For example, the disclosed compositions can be administered in an effective amount to induce, increase, or enhance an immune response. Immune response typically refers to responses that induce, increase, or perpetuate the activation or efficiency of innate and/or adaptive immunity. The compositions can also be used to promote tolerance, e.g., to an allergen or autoimmune antigen, rather than immunity.

The composition can be delivered parenterally (e.g., by subcutaneous, intradermal, or intramuscular injection) through the lymphatics, or by systemic administration through the circulatory system (e.g., by intravenous injection or infusion). In some embodiments, different compositions are administered in the same manner or route. In other embodiments, different compositions are administered in two or more different manners or routes.

The compositions can be delivered non-systemically. In some embodiments, at least the immunostimulatory agent-containing nanostructure alone or in combination with one or more additional agent is delivered locally, for example, by subcutaneous injection. In some embodiments, the composition is administered at a site adjacent to or leading to one or more lymph nodes which are close to the site in need of an immune response (i.e., close to a tumor or site of infection). The composition can be injected into the muscle. In some embodiments, the composition is administered in multiple doses at various locations throughout the body. The composition can also be administered directly to a site in need of an immune response (e.g., a tumor or site of infection).

A. Methods of Inducing an Immune Response, Immunity, and/or Antibody Production

Methods of inducing an immune response in a subject (e.g., a human) by administering to the subject a therapeutically effective amount of the disclosed compositions are provided. The immune response can be induced, increased, or enhanced by the composition compared to a control (e.g., absence of the composition or presence of another composition).

In some embodiments, the antigen-bound nanostructure functions as MHC with peptides to mimic dendritic cell presentation to T-cells for their activation and/or is subject to cleavage of antigen (e.g., neoantigenic peptides) by proteases to release them site-specifically, preferably in a prescribed stoichiometric amount; alone or in combination with inducing or enhancing immune cell activation through DNA duplex internalization and activation of e.g., STING/RIG, in, for example, a tumor microenvironment.

In some embodiments, the disclosed antigen-bound nanostructures increase a B cell response, for example increasing antigen binding to B cell receptors, increased antigen internalization by B cells, increased calcium release, increased pSyk phosphorylation, or a combination thereof. In some embodiments, B cell proliferation and/or differentiation is increased.

In some embodiments, a disclosed composition is administered to a subject in need thereof in an effective amount to increase an antigen-specific antibody response (e.g., IgG, IgG2a, IgG1, or a combination thereof), increase a response in germinal centers (e.g., increase the frequency of germinal center B cells, increase frequencies and/or activation T follicular helper (Tfh) cells, increase B cell presence or residence in dark zone of germinal center or a combination thereof), increase plasmablast frequency, increase inflammatory cytokine expression (e.g., IL-6, IFN-γ, IFN-α, IL-1β, TNF-α, CXCL10 (IP-10), or a combination thereof), or a combination thereof.

In some embodiments, the administration of the composition alternatively or additionally induces an improved B-memory cell response in subjects administered the composition compared to a control. An improved B-memory cell response is intended to mean an increased frequency of peripheral blood B lymphocytes capable of differentiation into antibody-secreting plasma cells upon antigen encounter.

The compositions can induce an improved effector cell response such as a CD4 or CD8 T-cell immune response, against at least one of the component antigen(s) or antigenic compositions compared to the effector cell response obtained under control conditions (e.g., absence of the composition or presence of another composition). The term “improved effector cell response” refers to a higher effector cell response such as a CD8 or CD4 response obtained in a human patient after administration of a disclosed composition than that obtained under control conditions. The improved effector cell response can be assessed by measuring, for example, the number of cells producing any of the following cytokines: (1) cells producing at least two different cytokines (CD40 μL, IL-2, IFN-gamma, TNF-alpha); (2) cells producing at least CD40 μL and another cytokine (IL-2, TNF-alpha, IFN-gamma); (3) cells producing at least IL-2 and another cytokine (CD40 μL, TNF-alpha, IFN-gamma); (4) cells producing at least IFN-gamma and another cytokine (IL-2, TNF-alpha, CD40L); (5) cells producing at least TNF-alpha and another cytokine (IL-2, CD40 μL, IFN-gamma); and (6) cell producing at least IFN-gamma.

The disclosed pharmaceutical compositions can be used, for example, to induce an immune response, when administering the immunostimulatory component alone or alternative compositions that are available (such as vaccines) is ineffectual. In some embodiments, the disclosed compositions may reduce the dosage of an immunostimulatory agent (e.g., antigen, adjuvant, or both) required to induce, increase, or enhance an immune response; or reduce the time needed for the immune system to respond following administration.

The described compositions may be administered as part of prophylactic vaccines or immunogenic compositions which confer resistance in a subject to subsequent exposure to infectious agents, or as part of therapeutic vaccines, which can be used to initiate or enhance a subject's immune response to a pre-existing antigen, such as a viral antigen in a subject infected with a virus or with cancer.

The desired outcome of a prophylactic or therapeutic immune response may vary according to the disease or condition to be treated, or according to principles well known in the art. For example, an immune response against an infectious agent may completely prevent colonization and replication of an infectious agent, affecting “sterile immunity” and the absence of any disease symptoms. However, a vaccine against infectious agents may be considered effective if it reduces the number, severity or duration of symptoms; if it reduces the number of individuals in a population with symptoms; or reduces the transmission of an infectious agent. Similarly, immune responses against cancer, allergens or infectious agents may completely treat a disease, may alleviate symptoms, or may be one facet in an overall therapeutic intervention against a disease.

The disclosed compositions may be used in methods of inducing protective immunity against an infectious agent, disease, or condition by administering to a subject (e.g., a human) a therapeutically effective amount of the compositions. “Protective immunity” or “protective immune response” refers to immunity or eliciting an immune response against an infectious agent, which is exhibited by a subject (e.g., a human), that prevents or ameliorates an infection or reduces at least one symptom thereof.

Another disclosed method provides for inducing the production of neutralizing antibodies or inhibitory antibodies in a subject (e.g., a human) by administering any of the disclosed compositions to the subject. In some embodiments, a disclosed composition is administered to a subject in need thereof in an effective amount to increase an antigen-specific antibody response (e.g., IgA, IgD, IgE, IgM, IgG, IgG2a, IgG1, or a combination thereof).

The antibody response is important for preventing many viral infections and may also contribute to resolution of infection. When a vertebrate (e.g., a human) is infected with a virus, antibodies are produced against many epitopes on multiple virus proteins. A subset of these antibodies can block virus infection by a process called neutralization. Antibodies can neutralize viral infectivity in a number of ways. They may interfere with virion binding to receptors (blocking viral attachment), block uptake into cells (e.g., blocking endocytosis), prevent uncoating of the genomes in endosomes, or cause aggregation of virus particles. Many enveloped viruses are lysed when antiviral antibodies and serum complement disrupt membranes.

### 1. Scaffold Enhanced Stimulation of Germinal Centers

In some forms, the nanoparticle scaffold is non-immunogenic per se, such that an humoral immune response to the described antigen-conjugated nanoparticles resulting from administering the nanoparticles to a host subject in vivo is primarily directed to the conjugated antigen, as opposed to the scaffold structure. Typically, in previous vaccine formulations the immune responses generated to both the antigen and the antigen-conjugated nanoparticle scaffold are thymus dependent, whereas in these compositions, the immune response generated to the conjugated antigen(s) will stimulate Germinal Centers (GC) through T-dependent pathways while there is no T-dependent immune response and GC to the antigen-conjugated nanoparticle scaffold. The lack of scaffold-directed immune response increases or enhances the Germinal Centers (GC) directed towards an antigen.

### B. Methods of Inducing Tolerance

The compositions and methods disclosed herein may also be used to promote tolerance. Tolerogenic therapy aims to induce immune tolerance where there is pathological or undesirable activation of the normal immune response. Such embodiments may also include co-administration of an immunosuppressive agent (e.g., rapamycin).

Tolerogenic vaccines deliver antigens with the purpose of suppressing immune responses (e.g., induce or increase a suppressive immune response) and promoting robust long-term antigen-specific immune tolerance. For example, Incomplete Freund's Adjuvant (IFA) mixed with antigenic peptides stimulates Treg proliferation (and/or accumulation) and IFA/Insulin peptide prevents type I diabetes onset in susceptible mice, though this approach is ineffective in reversing early onset type I diabetes (see, e.g., Fousteri, et al., Diabetologia, 53:1958-1970 (2010)).

The compositions and methods disclosed herein are also useful for controlling the immune response to an antigen. For example, in some embodiments, the compositions are used as part of a tolerizing vaccine.

An exemplary composition typically contains nucleic acid nanostructure with an antigen or a nucleic acid encoding an antigen, and an adjuvant. The antigen, for example, a self-antigen, depends on the disease to be treated, and can be determined by one of skill in the art. Exemplary self-antigens and other tolerizing antigens are discussed in more detail above. Adjuvant and antigen can be administered in an amount effective to, for example, to increase immunosuppression.

### C. Methods of Treatment

Also provided are methods of treating a subject (e.g., a human) having or at risk of having a disease or condition by administering to the subject a therapeutically effective amount of the disclosed compositions.

### 1. Infectious Diseases

The compositions are useful for treating acute or chronic infectious diseases. Thus, the compositions can be administered for the treatment of local or systemic viral infections, including, but not limited to, immunodeficiency (e.g., HIV), papilloma (e.g., HPV), herpes (e.g., HSV), encephalitis, influenza (e.g., human influenza virus A), and common cold (e.g., human rhinovirus) viral infections. For example, pharmaceutical formulations including the composition can be administered topically to treat viral skin diseases such as herpes lesions or shingles, or genital warts. The composition can also be administered to treat systemic viral diseases, including, but not limited to, AIDS, influenza, the common cold, or encephalitis.

Representative infections that can be treated, include but are not limited to infections cause by microorganisms including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Histoplasma, Hyphomicrobium, Legionella, Leishmania, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, Yersinia, Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Plasmodium vivax, Trypanosoma brucei, Entamoeba histolytica, Toxoplasma gondii, Trichomonas vaginalis and Schistosoma mansoni.

In some embodiments, the type of disease to be treated or prevented is a chronic infectious disease caused by a bacterium, virus, protozoan, helminth, or other microbial pathogen that enters intracellularly. In particular embodiments, infections to be treated are chronic infections caused by a hepatitis virus, a human immunodeficiency virus (HIV), a human T-lymphotrophic virus (HTLV), a herpes virus, an Epstein-Barr virus, or a human papilloma virus.

### 2. Cancer

The disclosed compositions are useful for treating cancer by, for example, stimulating or enhancing an immune response in host against the cancer. The types of cancer that may be treated with the provided compositions and methods include, but are not limited to, the following: bladder, brain, breast, cervical, colo-rectal, esophageal, kidney, liver, lung, nasopharangeal, pancreatic, prostate, skin, stomach, uterine, ovarian, testicular and hematologic.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The compositions can be administered as an immunogenic composition or as part of vaccine, such as prophylactic vaccines, or therapeutic vaccines, which can be used to initiate or enhance a subject's immune response to a pre-existing antigen, such as a tumor antigen in a subject with cancer.

The desired outcome of a prophylactic or therapeutic immune response may vary according to the disease, according to principles well known in the art. Similarly, immune responses against cancer, may alleviate symptoms, or may be one facet in an overall therapeutic intervention against a disease. For example, administration of the composition may reduce tumor size, or slow tumor growth compared to a control. The stimulation of an immune response against a cancer may be coupled with surgical, chemotherapeutic, radiologic, hormonal and other immunologic approaches in order to affect treatment. In some embodiments, the nanostructure itself or an additional active agent administered in combination therewith induces or enhances STING/RIG pathway activation (Baber, Nat Rev Immunol., 15(12):760-770 (2015)).

### 3. Inflammatory and Autoimmune Disorders

The compositions (e.g., those that increase tolerance) disclosed herein can be used to inhibit immune-mediated tissue destruction for example in a setting of inflammatory responses, autoimmune and allergic diseases, and transplant rejection.

In certain embodiments, the disclosed compositions are used to treat an inflammatory response or autoimmune disorder in a subject. For example, the disclosed methods can be used to prophylactically or therapeutically inhibit, reduce, alleviate, or permanently reverse one or more symptoms of an inflammatory response or autoimmune disorder. An inflammatory response or autoimmune disorder can be inhibited or reduced in a subject by administering to the subject an effective amount of a composition in vivo, or cells modulated by the composition ex vivo.

Representative inflammatory responses and autoimmune diseases that can be inhibited or treated include, but are not limited to, rheumatoid arthritis, systemic lupus erythematosus, alopecia areata, ankylosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Bechet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis - juvenile, discoid lupus, essential mixed cryoglobulinemia, fibromyalgia - fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

In some embodiments, the disclosed compositions and methods for inducing or perpetuating a suppressive immune response can be used prophylactically or therapeutically to suppress allergies and/or asthma and/or inflammation. Allergies and/or asthma and/or inflammation can be suppressed, inhibited or reduced in a subject by administering to the subject an effective amount of a composition that promotes an immune suppressive immune response or tolerance as described above.

4. Transplant rejection

In some embodiments, the disclosed compositions and methods can be used prophylactically or therapeutically to reduce or inhibit graft rejection or graft verse host disease. Transplant rejection occurs when a transplanted organ or tissue is not accepted by the body of the transplant recipient. Typically, rejection occurs because the immune system of the recipient attacks the transplanted organ or tissue. The disclosed methods can be used to promote immune tolerance of the transplant or graft by the receipt by administering to the subject an effective amount of a composition in vivo, or cells modulated by the composition ex vivo.

The transplanted material can be cells, tissues, organs, limbs, digits or a portion of the body, for example the human body. The transplants are typically allogenic or xenogenic. The disclosed compositions are administered to a subject in an effective amount to reduce or inhibit transplant rejection. The compositions can be administered systemically or locally by any acceptable route of administration. In some embodiments, the compositions are administered to a site of transplantation prior to, at the time of, or following transplantation.

In one embodiment, compositions are administered to a site of transplantation parenterally, such as by subcutaneous injection.

In other embodiments, the compositions are administered directly to cells, tissue or organ to be transplanted ex vivo. In one embodiment, the transplant material is contacted with the compositions prior to transplantation, after transplantation, or both.

In other embodiments, the compositions are administered to immune tissues or organs, such as lymph nodes or the spleen.

The transplant material can also be treated with enzymes or other materials that remove cell surface proteins, carbohydrates, or lipids that are known or suspected of being involved with immune responses such as transplant rejection.

5. Graft-versus-host disease (GVHD)

The disclosed compositions and methods can be used to treat graft-versus-host disease (GVHD) by administering an effective amount of the composition to alleviate one or more symptoms associated with GVHD. GVHD is a major complication associated with allogeneic hematopoietic stem cell transplantation in which functional immune cells in the transplanted marrow recognize the recipient as “foreign” and mount an immunologic attack. It can also take place in a blood transfusion under certain circumstances. Symptoms of GVD include skin rash or change in skin color or texture, diarrhea, nausea, abnormal liver function, yellowing of the skin, increased susceptibility to infection, dry, irritated eyes, and sensitive or dry mouth.

### D. Combination Therapies

In some embodiments, the compositions are administered in further combination with one or more additional therapeutic agents. The agents can be administered in the same or separate pharmaceutical composition from the nanostructure, antigen, adjuvant, or combinations thereof.

In some embodiments, the compositions are administered in combination with a conventional therapeutic agent used for treatment of the disease or condition being treated. Conventional therapeutics agents are known in the art and can be determined by one of skill in the art based on the disease or disorder to be treated. For example, if the disease or condition is cancer, the compositions can be co-administered with a chemotherapeutic drug; or if the disease or condition is a bacterial infection, the compositions can be co-administered with an antibiotic. When administered as a cancer vaccine, the disclosed compositions may be administered in combination with a checkpoint inhibitor (PD1, CTLA4, TIM3, etc.).

### E. Treatment Regimens

The disclosed compositions can be administered as a vaccine that includes a first (“prime”) and optionally one or more (“boost”) administrations. Thus in some embodiments, the composition is administered 2, 3, 4, or more times, for example, 1, 2, 3, 4, 5, 6, 7, 8, 9, 10, 11, 12, 13, 14, or 15 days, weeks, months, or years apart. Dosage regimens or cycles of the compositions and/or additional therapeutic agents can be completely or partially overlapping, or can be sequential.

F. Methods of Determining the Valency and Spatial Organization of Effective Presentation of Immunostimulatory agent

Also disclosed are methods of determining the most preferred valance and spatial organization of antigen presentation on nanostructure to, for example, induce an immune response.

An exemplary method of selecting a nucleic acid nanostructure can include assaying the ability of two or more structurally different antigen-bound nucleic acid nanostructure to induce an immune response, wherein the two or more structurally different nucleic acid nanostructures differ by (i) the structure of the antigen(s); (ii) the copy number of the antigen(s); (iii) spacing of the antigen(s); (iv) location of the antigen(s) on the nanostructure; (v) rigidity/flexibility of the antigen(s); (vi) dimensionality of the antigen(s); (vii) topology of the nanostructure; (viii) ultra-structural organization of the nanostructure; (ix) geometric shape of the nanostructure; or (x) a combination thereof.

For example, in some embodiments, the two or more structurally different nucleic acid nanostructures differ by (ii) and the copy number of antigen on each nanostructure is independently selected from 2 to 60 copies inclusive per nanostructure.

In some embodiment, the two or more structurally different nucleic acid nanostructures differ by (iii) and wherein the inter-antigen distance between adjacent antigens on each nanostructure is independently selected from between 3 nm to 80 nm inclusive.

In some embodiments, the two or more structurally different nucleic acid nanostructures differ by (ix), and the geometric shape of each nanostructure is independently selected from helix bundles, cuboidal structures, icosahedral structures, tetrahedral structures, cuboctahedral structures, octahedral structures, decahedral structures, and hexahedral structures.

Such methods can also include additional steps such as preparing the two or more nucleic acid nanostructure having a desired geometric shape, where the nanostructure is designed to allow for control of the relative position and/or stoichiometry of the antigen; covalently or non-covalently conjugating antigen to the surface of the nanostructure, wherein each of the two or more nanostructures vary by one or more of the structure of the antigen(s), the copy number of the antigen(s), spacing of the antigen(s), location of the antigen(s) on the nanostructure, rigidity/flexibility of the antigen(s), dimensionality of the antigen(s), topology of the nanostructure, ultra-structural organization of the nanostructure, and/or geometric shape of the nanostructure.

In some embodiments, one or more antigen-bound nanostructures having the highest or most desirable immune response is selected for use as an antigen in method of immunization or treatment such as those disclosed herein.

Any one or more of the factors (i)-(ix) can be varied systematically or non-systematically. In some embodiments, 2, 5, 10, 25, 50, 100, or more different antigen-bound nanostructures are tested.

In some embodiments, where two or more nanostructures induce the same or similar immune responses, the nanostructure that is the simplest to make, the nanostructure that is the least expensive to make, or a combination thereof is selected for use as an antigen in a method of immunization or treatment such as those disclosed herein.

The disclosed compositions and methods can be further understood through the following numbered paragraphs.
1. A nucleic acid nanostructure including a defined geometric shape and two or more copies of an antigen bound to the surface of the nanostructure, wherein the number of copies of the antigen, the distance between adjacent copies of the antigen, the location of the antigen on the nanostructure, the rigidity/flexibility of the antigen, the dimensionality of the antigen, the topology of the nanostructure, the ultra-structural organization of the nanostructure, the geometric shape of the nanostructure, or a combination thereof improves an immune response induced by the antigen relative to a control nucleic acid nanostructure including at least one copy of the same antigen.
2. The nanostructure of paragraph 1, wherein the number of copies of the antigen is 2 to 100, or 3 to 75, or 4 to 60, or 30 to 60, or 5 to 50, or 10 to 50, or 5 to 25, or 5 to 10 inclusive.
3. The nanostructure of paragraph 2, wherein the number of copies of the antigen is 5, 6, 7, 8, 9, 10, 15, 20, 25, 30, 35, 40, 45, 50, 55, or 60.
4. The nanostructure of any one of paragraphs 1-3, wherein the distance between adjacent copies of the antigen is 1 nm to 150 nm, or 10 nm to 80 nm, or 15 nm to 50 nm, or 25 nm to 30 nm, or 2 nm to 15 nm, or 4 nm to 12 nm, or 4 nm to 10 nm, or 4 nm to 8 nm, inclusive.
5. The nanostructure of any one of paragraphs 1-4, wherein the geometric shape is selected from the group including a helix bundle, cuboidal structure, icosahedral structure, tetrahedral structure, cuboctahedral structure, octahedral structure, and hexahedral structure.
6. The nanostructure of any one of paragraphs 1-4, wherein the geometric shape is a polyhedron or a 6-helix bundle.
7. The nanostructure of paragraph 6, wherein the geometric shape is a polyhedron, and wherein the polyhedron is an icosahedron.
8. The nanostructure of any one of paragraphs 1-7, wherein the copies of the antigen are covalently or non-covalently bound to the nanostructure.
9. The nanostructure of any one of paragraphs 1-8, wherein the antigen is indirectly or directly bound to the nanostructure via outwardly facing nucleic acid overhangs extending from the 3′ or 5′ ends of selected staple strands of the nanostructure.
10. The nanostructure of any one of paragraphs 1-9, wherein the nucleic acid is DNA.
11. The nanostructure of paragraph 10, wherein the nucleic acid overhangs hybridize to the complementary target RNA, DNA or PNA covalently linked to the antigen.
12. The nanostructure of paragraph 11, wherein the covalent linkage is formed by maleimide-thiol coupling.
13. The nanostructure of any one of paragraphs 1-12, including two or more structurally different antigens.
14. The nanostructure of paragraphs 1-13, wherein the antigen(s) is associated with one or more diseases or conditions selected from infectious diseases, autoimmune diseases, and cancer.
15. The nanostructure of any one of paragraphs 1-14, wherein the antigen is an HIV immunogen.
16. The nanostructure of any one of paragraphs 1-15, wherein the antigen binds to a broadly neutralizing antibody.
17. The nanostructure of paragraph 16, wherein the antigen is an HIV gpl20 epitope.
18. The nanostructure of paragraph 17, wherein the epitope encompasses the CD4 binding site of gp120.
19. The nanostructure of paragraphs 1-18, wherein the antigen is selected from eOD-GT6, eOD-GT8, or variants thereof.
20. The nanostructure of any one of paragraphs 1-19 further including one or more moieties incorporated in and/or linked to the nanostructure.
21. The nanostructure of paragraph 20, wherein one or more of the moieties is an adjuvant.
22. The nanostructure of paragraph 20 or 21, wherein one or more of the moieties is a targeting molecule.
23. The nanostructure of any one of paragraphs 20-22, wherein one or more of the moieties is a therapeutic agent.
24. The nanostructure of any one of paragraphs 1-23, wherein the nanostructure is coated with a coating agent.
25. The nanostructure of paragraph 24, wherein the coating agent is a naturally occurring or synthetic cationic oligomer or polymer or cooligomer, optionally including or including PEG moieties.
26. The nanostructure of paragraphs 24 or 25 wherein the coating agent includes or consists of (a) 10 lysine units optionally conjugated terminally to a linear PEG moiety, optionally wherein the PEG has a molecular weight of approximately 5000 Da; (b) poly(2-dimethylaminoethyl methacrylate (PDMAEMA) or a PEG copolymer thereof optionally having a molecular weight range between 5000 Da to 20000 Da; (c) linear polyethyleneimine (PEI), optionally, in a molecular weight range between 5000 Da and 10000 Da; (iv) chitosan optionally a molecular weight range between 4000 Da and 6000 Da optionally with deacetylation of more than 90%.
27. The nanostructure of paragraphs 24 or 25, wherein the coating agent includes or consists of a minor groove binder.
28. The nanostructure of paragraph 27, wherein the minor groove binder increases stabilization, optionally wherein the stabilization includes protecting the protection for endonuclease.
29. The nanostructure of paragraphs 27 and 28, wherein the minor groove binder is selected from bisamidines, polyamides, bisbenzimidazoles and combinations thereof.
30. The nanostructure of any one of paragraphs 1-29, wherein the nanostructure is an icosahedron including 5 to 50 copies of the antigen, and wherein the copies of the antigen are spaced 10 nm to 40 nm inclusive apart.
31. The nanostructure of any one of paragraphs 1-29, wherein the nanostructure is an a pentagonal bipryamidal structure including 10 to 40 copies of the antigen, and wherein the copies of the antigen are spaced 10 nm to 40 nm inclusive apart.32. A pharmaceutical composition including the nucleic acid nanostructure of any one of paragraphs 1-31 and a pharmaceutically acceptable carrier, diluent, preservative, excipient, or combination thereof.
33. The pharmaceutical composition of paragraph 32, further including a coating agent.
34. The pharmaceutical composition of paragraph 33, wherein the coating agent is the same or different from the coating agent of any one of paragraphs 26-29.
35. The pharmaceutical composition of any one of paragraphs 32-34, wherein the nucleic acid nanostructure is present in an effective amount to induce an immune response in a subject in need thereof, with or without the aid of an adjuvant.
36. The pharmaceutical composition of paragraph 35, further including an adjuvant in an effective amount to enhance the immune response relative to administration of the nucleic acid nanostructure alone.
37. A method of inducing an immune response in a subject including administering to the subject an effective amount of the nucleic acid nanostructure of any one of paragraphs 1-31 or the pharmaceutical composition of any one of paragraphs 32-36 alone or in combination with an adjuvant.
38. A method of vaccinating or otherwise inducing protective immunity against an infectious agent, disease, or condition including administering to the subject an effective amount of the nucleic acid nanostructure of any one of paragraphs 1-31 or the pharmaceutical composition of any one of paragraphs 32-36 alone or in combination with an adjuvant.
39. A method of treating a subject having or at risk of having a disease or condition including administering to the subject an effective amount of the nucleic acid nanostructure of any one of paragraphs 1-31 or the pharmaceutical composition of any one of paragraphs 32-36 alone or in combination with an adjuvant.
40. A method of inducing the production of neutralizing antibodies or inhibitory antibodies in a subject including administering to the subject an effective amount of the nucleic acid nanostructure of any one of paragraphs 1-31 or the pharmaceutical composition of any one of paragraphs 32-36 alone or in combination with an adjuvant.
41. The method of any one of paragraphs 37-38, wherein the subject is a human.
42. A method of selecting a nucleic acid nanostructure including assaying the ability of two or more structurally different antigen-bound nucleic acid nanostructure to induce an immune response, wherein the two or more structurally different nucleic acid nanostructures differ by
   (i) the structure of the antigen(s);
   (ii) the copy number of the antigen(s);
   (iii) spacing of the antigen(s);
   (iv) location of the antigen(s) on the nanostructure;
   (v) rigidity/flexibility of the antigen(s);
   (vi) dimensionality of the antigen(s);
   (vii) topology of the nanostructure;
   (viii) ultra-structural organization of the nanostructure;
   (ix) geometric shape of the nanostructure; or
   (x) a combination thereof.
43. The method of paragraph 42, wherein the two or more structurally different nucleic acid nanostructures differ by (ix), and wherein the geometric shape of each nanostructure is independently selected from helix bundles, cuboidal structures, icosahedral structures, tetrahedral structures, cuboctahedral structures, octahedral structures, and hexahedral structures.
44. The method of paragraphs 32 or 43, wherein the two or more structurally different nucleic acid nanostructures differ by (ii) and wherein the copy number of antigen on each nanostructure is independently selected from 2 to 60 copies per nanostructure.
45. The method of any one of paragraphs 42-44, wherein the two or more structurally different nucleic acid nanostructures differ by (iii) and wherein the inter-antigen distance between adjacent antigens on each nanostructure is independently selected from 3 nm to 80 nm.
46. A nucleic acid nanostructure including a defined geometric shape and one or more copies of an antigen that can induce an immune response against human immunodeficiency virus (HIV).
47. The nanostructure of paragraph 46, wherein the antigen binds to a broadly neutralizing antibody.
48. The nanostructure of paragraph 47, wherein the antigen is an HIV gp120 epitope.
49. The nanostructure of paragraph 48, wherein the epitope encompasses the CD4 binding site of gp120.
50. The nanostructure of any of paragraphs 46-49, wherein the antigen is selected from eOD-GT6, eOD-GT8, or variants thereof.
51. A nucleic acid nanostructure including a defined geometric shape and one or more copies of an antigen derived from a H-2Kk MHC class I molecule.
52. The nucleic acid nanostructure of paragraph 51, wherein the antigen is a p31 peptide or a p5 peptide.
53. The nanostructure of any one of paragraphs 46-52 including 2 to 50 or 10 to 50, or 10 to 40, or 2 to 20 or 5 to 10 copies of the antigen inclusive.
54. The nanostructure of paragraph 53 including 5 to 10 copies of the antigen inclusive.
55. The nanostructure of paragraphs 53 or 54 wherein adjacent antigens are separated by an inter-antigen distance of 5 nm to 80 nm inclusive.
56. The nanostructure of paragraph 55, wherein adjacent antigens are separated by an inter-antigen distance of at least 28 nm.
57. The nanostructure of any one of paragraphs 46-56, wherein the geometric shape is a helix bundle, cuboidal structure, icosahedral structure, decahedron structure, tetrahedral structure, cuboctahedral structure, octahedral structure, or hexahedral structure.
58. The nanostructure of any one of paragraphs 46-57, wherein the geometric shape is a polyhedron or a 6-helix bundle.
59. The nanostructure of paragraph 58, wherein the geometric shape is a polyhedron, and wherein the polyhedron is an icosahedron.
60. The nanostructure of any one of paragraphs 46-57, wherein the genometric shape is a pentagonal bipyramid.
61. An immunogen for inducing an immune response against HIV including icosahedron-shaped DNA nanostructure including 5-50 copies or 10-50 or 5-10 copies of eOD-GT8 antigen, wherein each copy of the antigen is linked to the nanostructure by a single stranded peptide nucleic acid conjugated directly to the antigen and hybridized to a complementary sequence at the 3′ single stranded overhang of a staple strand of the nanostructure, and wherein each copy of the antigen is spaced from the other copies of antigen by at least 5 nm, 10 nm, 15 nm, 20 nm, 25 nm, 28 nm, or 30 nm and optionally not more than 100 nm.
62. An immunogen for inducing an immune response again HIV including pentagonal bipyramid-shaped DNA nanostructure including 10-50 copies or 10-40 copies inclusive of eOD-GT8 antigen, wherein each copy of the antigen is linked to the nanostructure by a single stranded peptide nucleic acid conjugated directly to the antigen and hybridized to a complementary sequence at the 3′ single stranded overhang of a staple strand of the nanostructure, and wherein each copy of the antigen is spaced from the other copies of antigen by at least 5 nm, 10 nm, 15 nm, 20 nm, 25 nm, 28 nm, or 30 nm and optionally not more than 100 nm.
63. The nanostructure of any one of paragraphs 46-60 or the immunogen of paragraphs 61 or 62 wherein the nanostructure or immunogen is coated with a coating agent.
64. The nanostructure or immunogen of paragraph 63, wherein the coating agent is a naturally occurring or synthetic cationic oligomer or polymer or cooligomer, optionally including or including PEG moieties.
65. The nanostructure or immunogen of paragraphs 63 or 64, wherein the coating agent includes or consists of (a) 10 lysine units optionally conjugated terminally to a linear PEG moiety, optionally wherein the PEG has a molecular weight of approximately 5000 Da; (b) poly(2-dimethylaminoethyl methacrylate (PDMAEMA) or a PEG copolymer thereof optionally having a molecular weight range between 5000 Da to 20000 Da; (c) linear polyethyleneimine (PEI), optionally, in a molecular weight range between 5000 Da and 10000 Da; (iv) chitosan optionally a molecular weight range between 4000 Da and 6000 Da optionally with deacetylation of more than 90%.
66. The nanostructure or immunogen of paragraphs 63 or 64, wherein the coating agent includes or consists of a minor groove binder.
67. The nanostructure or immunogen of paragraph 66, wherein the minor groove binder increases stabilization, optionally wherein the stabilization includes protecting the protection for endonuclease.
68. The nanostructure or immunogen of paragraphs 66 and 67, wherein the minor groove binder is selected from bisamidines, polyamides, bisbenzimidazoles and combinations thereof.
69. A pharmaceutical composition including the nanostructure or immunogen of any one of paragraphs 46-68, a pharmaceutically acceptable carrier, and optionally an adjuvant.
70. The pharmaceutical composition of paragraph 69 further including a coating agent.
71. The pharmaceutical composition of paragraph 70, wherein the coating agent is coating agent of any one of paragraphs 63-68.
72. A method of treating a subject in need thereof including administering to the subject an effective amount of the pharmaceutical composition of any one of paragraphs 69-71 to induce an immune response in the subject.
73. The method of paragraph 72, wherein the subject has HIV, the antigen is eOD-GT8, and the immune response is again HIV.
74. A method of vaccinating a subject against HIV including administering the subject an effective amount of the pharmaceutical composition of any one of paragraphs 69-71 to induce an immune response against eOD-GT8. 101. A nucleic acid nano-structured virus like particle (NANVLP) for stimulation of germinal center B cells (GCB), including:
   (i) a nucleic acid nanostructure (NAN);
   (ii) a plurality of antigen molecules, wherein the plurality of antigen or engineered immunogen molecules are displayed on the NAN surface; and
   (iii) optionally one or more helper T cell epitope designed to elicit GCB; and
   (iv) optionally densely glycosylated antigens or synthetic oligomannose structures conjugated to NANVLP surface for complement system activation, wherein the plurality of antigen molecules and optionally the one or more T cell epitope are configured for stimulation of antigen-specific B cells.
102. The NANVLP of paragraph 101, wherein the plurality of antigen molecules are present on the NAN surface at a density of between about 0.04 and about 0.14 molecules/nm², inclusive.
103. The NANVLP of paragraph 101, wherein the plurality of antigen molecules are evenly distributed on the NAN surface, and
   wherein the distance between two molecules of the plurality of antigen molecules on the NAN surface is between about 10 nm and about 4 nm, inclusive.
104. The NANVLP of paragraph 101, wherein the NAN includes a single stranded nucleic acid scaffold sequence and a plurality of single stranded nucleic acid staple strands that hybridize to the scaffold sequence to form the three-dimensional nanostructure having a defined geometric shape.
105. The NANVLP of paragraph 104, wherein the geometric shape is selected from the group including a helix bundle, cuboidal structure, icosahedral structure, tetrahedral structure, cuboctahedral structure, octahedral structure, and hexahedral structure.
106. The NANVLP of paragraph 101, wherein, wherein the NAN has a diameter of between about 20 nm and about 100 nm, inclusive.
107. The NANVLP of paragraph 106, wherein, wherein the NAN has a diameter of from about 20 nm to about 30 nm, inclusive.
108. The NANVLP of paragraph 106, wherein the plurality of antigen includes from 10 to 200 molecules of antigen, inclusive.
109. The NANVLP of paragraph 106, wherein the plurality of antigen includes 60 molecules of antigen.
110. The nanostructure of paragraph 101, wherein the plurality of antigen molecules and/or the one or more T cell epitope are covalently or non-covalently bound to the NAN.
111. The nanostructure of paragraph 110, wherein the plurality of antigen molecules and/or the one or more T cell epitope are indirectly or directly associated with the NAN via nucleic acid overhangs extending from the 3′ or 5′ ends of one or more selected staple strands of the nanostructure or via covalent conjugation chemistries.
112. The NANVLP of paragraph 101, wherein all the antigen molecules of the plurality of antigen molecules are the same.
113. The NANVLP of paragraph 101, wherein all the antigen molecules of the plurality of antigen molecules are not the same.
114. The NANVLP of paragraph 101, wherein an antigen molecule of the plurality of antigen molecules is derived from the group including a protozoan, a bacterium, a virus, a fungus, and a cancer.
115. The NANVLP of paragraph 101, wherein an antigen molecule of the plurality of antigen molecules is derived from a virus, optionally wherein the virus is selected from the group including influenza, dengue viruses, Hepatitis C virus, picornaviruses, coronaviruses and human immunodeficiency virus (HIV).
116. The NANVLP of paragraph 101, wherein an antigen molecule of the plurality of antigen molecules is HIV immunogens engineered outer domain (eOD), core-g28v2, or SOSIP trimers.
117. The NANVLP of paragraph 101, wherein synthetic high mannose glycans are positioned at density of 0.04 and about 1 molecules/nm² independently of protein antigen.
118. The NANVLP of paragraph 101, including one or more helper T cell epitope.
119. The NANVLP of paragraph 118, wherein the T cell epitope is conjugated to or complexed with the antigen molecule.
120. The NANVLP of paragraph 119, wherein each antigen molecule of the plurality of antigen molecules is conjugated to a T cell epitope designed to elicit GCB.
121. The NANVLP of paragraph 118, wherein the T cell epitope is either synthetic or naturally derived and binds a broad range of HLA-DR alleles.
122. The NANVLP of paragraph 118, wherein the T cell epitope is a pan HLA DR-binding epitope (PADRE) peptide,
   optionally wherein the PADRE peptide includes the amino acid sequence AKFVAAWTLKAAA.
123. A thymus-independent nucleic acid nano-structured virus like particle (NANVLP) for stimulation of germinal center B cells (GCB), including:
   (i) a nucleic acid nanostructure (NAN) including a single stranded nucleic acid scaffold sequence and a plurality of single stranded nucleic acid staple strands that hybridize to the scaffold sequence to form the three-dimensional nanostructure having a defined icosahedral shape of about 23 nm;
   (ii) 60 antigen molecules linked to the NAN,
   wherein the antigen molecules are displayed on the NAN surface at a density of about 0.14 molecules/nm² and with an inter-antigen distance of between about 4 nm and about 6 nm, inclusive, and
   wherein each antigen molecule is conjugated to a PADRE polypeptide or other T cell epitope.
124. The NANVLP of paragraph 123, wherein the antigen includes HIV eOD-GT8,
   optionally wherein the eOD-GT8 molecules are covalently linked to the NAN,
   optionally wherein the covalent linkage is formed by maleimide-thiol coupling, strain-promoted azide-alkyne cycloaddition, or inverse electron-demand diels-alder reactions.
125. A pharmaceutical formulation including the NANVLP of paragraph 101, and a pharmaceutically acceptable excipient for administration in vivo.
126. A vaccine including the pharmaceutical formulation of paragraph 125,
   optionally further including an adjuvant.
127. A method for generating an immune response in vivo against a sub-dominant epitope, including administering to a subject the vaccine of paragraph 126.
128. The method of paragraph 127, wherein the vaccine is administered in an effective amount to increase an antigen-specific antibody response, or increase a response in epitope-specific germinal center B cell frequency, or increase plasmablast frequency, or increase memory B cell frequency, or increase somatic hypermutation rates of these B cells, or increase inflammatory cytokine expression, or a combination thereof in the subject as compared to a control, non NAN-based vaccine against the same antigen.
129. The method of paragraph 128, wherein the antigen-specific antibody response includes increasing or stimulating one or more of antigen specific IgG antibodies selected from group including, IgG1, IgG2, IgG3 and IgG4, or a combination thereof.
130. The method of paragraph 128, wherein the increase of a response in germinal centers includes increasing frequency and counts of epitope-specific germinal center B cells, increasing frequencies and/or activation T follicular helper (Tfh) cells, increasing B cell proliferation or residence in dark zone of germinal centers, increasing somatic hypermutation, or a combination thereof.
131. The method of paragraph 128, wherein the expression of inflammatory cytokines includes an increase or stimulation of expression of one or more cytokine selected from the group including IL-6, IL-21, IFN-γ, IFN-α, IL-1β, TNF-α, and CXCL10 (IP-10).
132. The method of paragraph 127, wherein the immune response further includes reducing undesired competitor B cell responses as compared to the control vaccine.

U.S. Ser. No. 63/333,498, U.S. Ser. No. 17/819,204, published as US-2023-0330219-A1, U.S. Ser. No. 62/796,472, U.S. Ser. No. 16/752,394, published as U.S. Published Application No. 2020/0237903, and PCT/US2020/014957, published as WO 2020/154595 are each specifically incorporated by reference herein in their entireties.

### EXAMPLES

### Example 1: DNA Nanoparticles can Efficiently Organize and Present Antigens

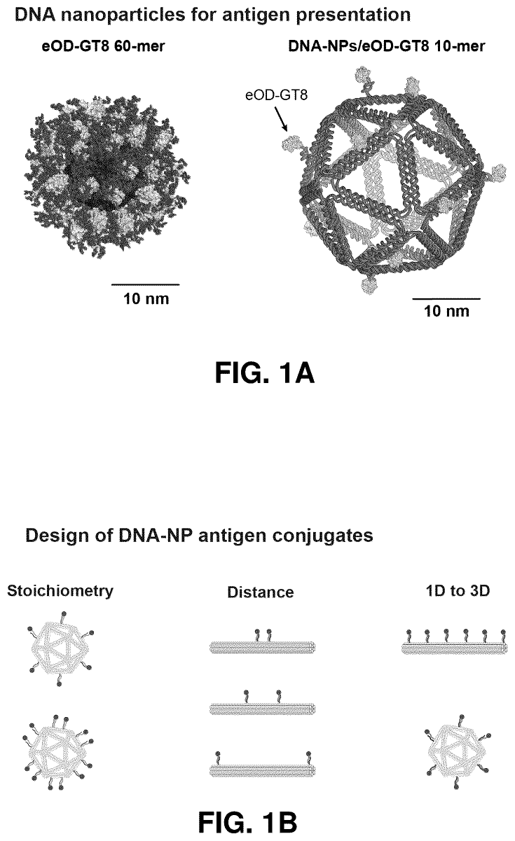

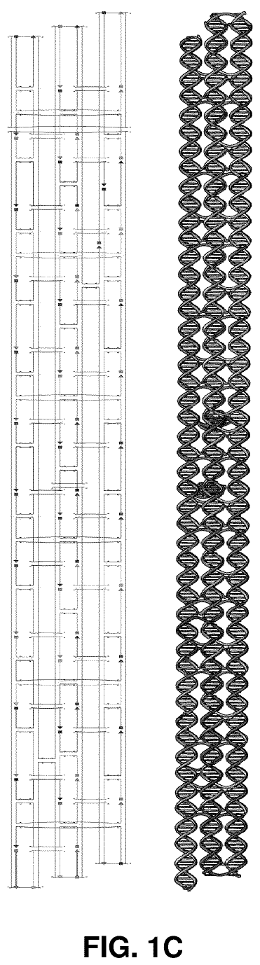

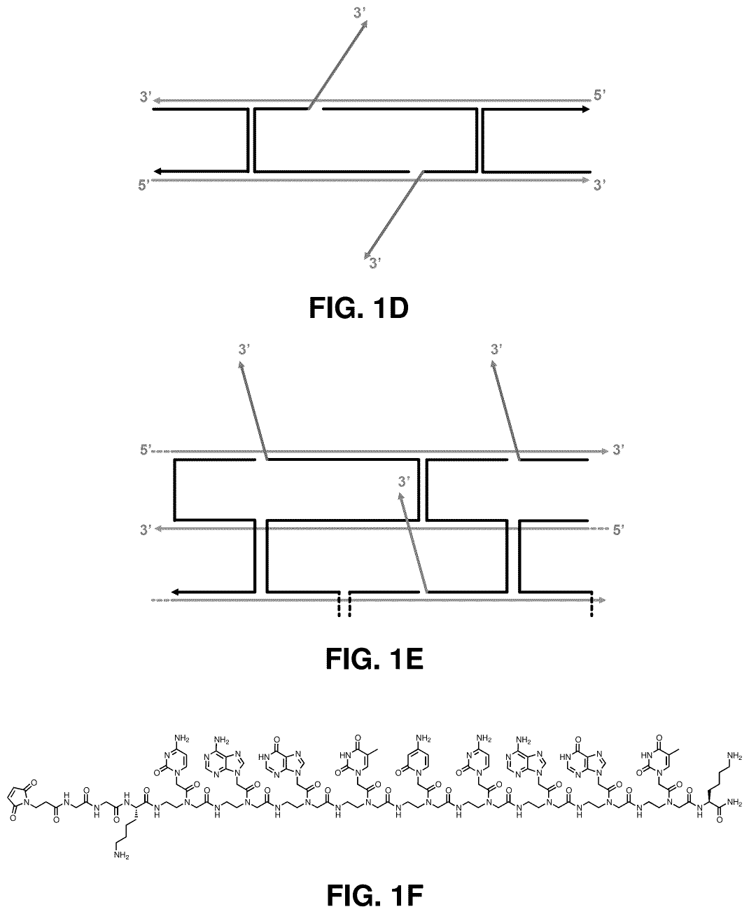

The ultra-structural parameters of displayed antigen that drive efficient B cell responses were evaluated in a DNA nanostructure model. Previously, eOD-GT8 and eOD-GT6 displayed in a self-assembled 60-mer protein nanoparticle elicited robust B cell activation both in vitro against VRCO1+B cells and in engineered mouse models (Jardine, et al., Science, 349(6244):156-161 (2015), Jardine, et al., Science, 340(6133):711-6 (2013), Jardine, et al., Science, 351(6280):1458-63 (2016)). Here, in order to determine which features (namely the stoichiometry, inter-antigen distances, substrate flexibility, and ultra-structural organization) of eOD-GT8 60-mer drive efficient B cell responses, a viral-like icosahedral DNA nanoparticle (DNA-NP; Veneziano, et al., Science, 352(6293):1534 (2016)) of size and shape similar to the eOD-GT8 60-mer was designed and assembled (FIG. 1A). eOD-GT8 was displayed on engineered DNA origami nanoparticles (Venziano, et al., Science, 352(6293):1534 (2016))), which allow for site specific and stoichiometric control over antigen conjugation through base pairing between engineered single stranded DNA (ssDNA) overhangs on the DNA nanoparticle and complementary peptide nucleic acid (PNA) strands site-specifically conjugated to eOD monomers. Using icosahedral DNA-NPs as well as rigid 6-helical DNA rods (6HB) (FIG. 1C), flexible ssDNA, and polyethylene glycol (PEG) polymer templates, the number of eOD-GT8 antigens per particle, the distance between these antigens, the topology or dimensionality of antigen presentation, and the flexibility of the scaffold used for presentation of the antigens were varied (FIG. 1B).

### Methods and Materials

Chemicals and kits

Magnesium chloride, TRIS acetate EDTA (TAE) buffer, TRIS-base, sodium chloride, Phosphate Buffer Saline, and Amicon ultra 0.5 centrifugal filter were provided by Sigma-Aldrich. Nuclease free water was provided by Integrated DNA Technologies (IDT). The DNTPs mix, the DNA ladder (Quick-Load® Purple 2-Log DNA ladder 0.1-10 kb) were provided by New England Biolabs (NEB), The polymerase enzyme (Accustart Taq DNA polymerase HiFi) was provided by Quanta Biosciences. Low melt agarose was purchased from IBI Scientific and agarose by Seakem. G-capsule for electroelution was provided by G-Biosciences and Freeze 'N Squeeze DNA gel extraction columns by Bio-rad. The Zymoclean Gel DNA recovery kit was purchased from Zymo Research. The SybrSafe DNA staining reagent was provided by ThermoFisher.

### Oligonucleotides and DNA Templates

All oligonucleotides used for asymmetric PCR (aPCR) amplification of template and for folding of the various DNA nanostructures were purchased from IDT. The circular plasmid DNA scaffold M13mp18 used for amplification of the short scaffold with aPCR was provided by NEB (#N4040S).

### Antigens and Cell Lines

The eOD antigen with an N-terminal cysteine was produced in HEK cells, and purified by affinity chromatography using a Nickel affinity column. The protein was then further purified by size exclusion chromatography using a Superdex 75 10/300 colum (GE Healthcare). Ramos B cells expressing VRC01 germline B cell receptor were provided by Daniel Lingwood (Ragon Institute).

ssDNA Scaffold Synthesis

The ssDNA scaffolds used to fold the DNA 6 helix bundle (6-HB) and the DNA icosahedron were produced using a previously described method of asymmetric PCR (Venziano, et al., Science, 352(6293):1534 (2016), Veneziano, et al., Sci. Rep., 8(1):6548 (2018)). Briefly, two specific primers sets were used to amplify the ssDNA fragments (Table 1) with Quanta Accustart HiFi DNA polymerase. The aPCR mix was prepared in a final volume of 50 μL with the specific polymerase buffer complemented with 2 mM of Magnesium chloride, 200 μM of dNTPs, 1 μM of forward primer, 20 nM of reverse primer, 25 ng of M13mp18 template and 1 unit of Quanta Accustart HiFi polymerase. The amplification program used is the following: 94° C., 1 min for the initial denaturation; followed by 35 cycles of 94° C., 20 sec; 56° C., 30 sec; 68° C., 1 min per kb to amplify. Following amplification the aPCR mix were run on a 1% low melt agarose gel prestained with SybrSafe and the ssDNA product was extracted using the Zymoclean gel DNA recovery kit. Purified ssDNA concentration was measured using NanoDrop 2000.

TABLE 1
List of primers used for amplification
of the DNA nanostructures

| | 5′-primer | 3′-primer |
|---|---|---|
| Structure | (forward) | (reverse) |
| 6-HB | CCCTTTAGGGTTC | GCTGAAAAGG |
| | CGATTTA | TGGCATCAAT |
| | (SEQ ID NO: 1) | (SEQ ID NO: 3) |
| Icosahedron | TCTTTGCCTTGCC | GCTAACGAGC |
| | TGTATGA | GTCTTTCCA |
| | (SEQ ID NO: 2) | (SEQ ID NO: 4) |

### DNA Origami Nanostructure Folding

The DNA nanoparticles (Icosahedron and 6-HB) with or without overhangs were assembled in a one-pot reaction annealing as described previously (Venziano, et al., Science, 352(6293):1534 (2016). Briefly, 20-40 nM of scaffold was mixed with an excess of the correct staple strand mix (molar ratio of 10×) in buffer TAE-MgCl2 (40 mM Tris, 20 mM acetic acid, 2 mM EDTA, 16 mM MgCl2, pH 8.0) in a final reaction volume of 50 μL and annealed with the following program: 95° C. for 5 min, 80-75° C. at 1° C. per 5 min, 75-30° C. at 1° C. per 15 min, and 30-25° C. at 1° C. per 10 min.

### DNA DX-Tile Nanostructure Folding

Each strand of the DX-tile was mixed at an equimolar concentration (2μM) and the same protocol of annealing was used as for origami folding. No further step of purification was needed for the DX-tile as the folding yield was close to 100%.

### Purification of DNA Origami Nanostructures

The DNA origami folded with an excess of staples strands were purified using Amicon ultra 0.5 centrifugal filter with three washes of folding buffer or PBS buffer if needed for further applications. Centrifugation steps were performed at 1000g for 30-40 minutes and the final concentration of nanostructures was determined using NanoDrop 2000.

PNA Strands Synthesis PNA strands were synthesized manually by solid phase peptide synthesis. Lysine residues were attached at either end of the PNA sequence to improve solubility. Fmoc-PNA monomers (PNA-Bio) were coupled to a low loading Tentagel-S-RAM resin using 4 eq. PNA, 3.95 eq. PyBOP, and 6 eq. diisopropylethylamine (DIEA). Lysine and glycine residues were reacted in the same way. Following each coupling, the peptide was deprotected in 20% piperidine in DMF. N-maleoyl-β-alanine (Sigma) was coupled to the N-terminus under the same coupling conditions. The peptide was then cleaved from the resin in 95% trifluoroacetic acid (TFA), 2.5% H₂O, and 2.5% triisopropylsilane. The peptide was dissolved in an aqueous solution with 0.1% TFA, filtered, and purified by HPLC using a C-18 Gemini column (Phenomenex) with a mobile phase of acetonitrile containing 0.1% TFA. Purity of the PNA products was analyzed with MALDI-TOF mass spectrometry on a Bruker Daltonics microflex.

Antigen Modification with PNA

PNA strands were attached to eOD by reacting the maleimide onto an N-terminal cysteine of eOD. Prior to the reaction, eOD was incubated with a 10-fold molar excess of tris(2-carboxyethyl)phosphine (TCEP) for 15 minutes, and TCEP was removed using centrifugal filter. Immediately after removal of TCEP, a 2-fold molar excess of maleimide-PNA was reacted with cysteine-eOD overnight at 4C in PBS. Unreacted PNA was then removed using an Amicon centrifugal filter (10 kDa MWCO).

### Antigen Attachment to DNA Nanostructures

Purified DNA nanostructures were mixed with the PNA-antigen conjugates at a molar ratio of 5× of antigen to overhangs on DNA nanostructures. An annealing ramp is realized starting from 37C to 4C at 1C for 20 min.

### Structure Characterization by TEM

DNA origami nanoparticles were visualized by transmission electron microscopy (TEM), with grids prepared as described previously with minor modifications. Briefly, carbon supported grids with copper mesh (CF200H-CU; Electron Microscopy Sciences) were glow discharge and soaked in 100 μM MgCl2 and blotted prior to applying DNA origami nanoparticles. 20 ul of 10 nM nanoparticle solution was applied to a clean parafilm surface and the grid was floated for 2 minutes. While soaking, 2% uranyl formate (UF; Electron Microscopy Sciences) was neutralized with 25 mM NaOH final concentration, vortexed for 1 minute, and filtered via syringe through a 0.1 μm filter (EMD Millipore) dropwise onto the clean parafilm surface. The grid was then removed and quickly dried by edge blotting with Whatman 44 ashless paper. The grid was then immediately transferred to the 2% UF solution and incubated for 30 seconds. Again, the grid was dried by blotting along the edge with Whatman paper, and left to dry in air for an additional 30 minutes prior to imaging. Imaging was done on a FEI Tecnai set to 120 kV with and Gatan camera. Images were taken at 6,500× for wide-field views and 52,000× for near-field views. Images were collected from 3-second exposures. All images were cropped in Adobe Photoshop with subsequent autocontrast applied.

### Agarose Gel Electrophoresis

DNA-nanoparticles folded and conjugated with eOD-GT8-PNA were analyzed by agarose gel electrophoresis with 2% agarose gel pre-stained with SybrSafe. Samples were loaded at a concentration of 20 to 50 nM DNA-NPs, ran for 2-3 hours at 90 V at 4C and visualized with a blue light transilluminator. For fluorescence gel analysis with the AF647 modified eOD-GT8, the image was take in a Typhoon FLA 7000 at the SybrSafe excitation wavelength (473 nm), and at the AF647 excitation wavelength (635 nm). Images were merged with the ImageJ software (3)

### Fluorescent Quantification

Quantification of the eOD-GT8 conjugation to DNA-NPs was performed with a fluoromax-4 (Horiba Inc.) fluorimeter. eOD-GT8-PNA monomer was modified with AF647 dye using NHS-NH₂ chemistry and used for conjugation to DNA-nanoparticles. Spectra were acquired with an excitation wavelength of 630 nm (emission measured at 670 nm). A fluorescence calibration curve was realized with free eOD-GT8-PNA, conjugated with AF647 dye, at different concentrations and used to determine yield of coverage of DNA-NPs.

### B Cell Calcium Flux Assay

Ramos B Cells at a concentration of 10 million cells/mL were incubated with 10 M Fluo-4 AM (Thermo Fisher) for 30 minutes at 37C. After washing once, flux assays were performed on a Teican plate reader at 37C on a 96 well microplate. 160 μL of Fluo-4 labeled Ramos cells at 2 million cells/mL was added to each well. A baseline fluorescence was then recorded for 1 minute, and 40 μL of nanoparticles were added to the cells for a final concentration of 5 nM of antigen, unless otherwise stated.

### Results

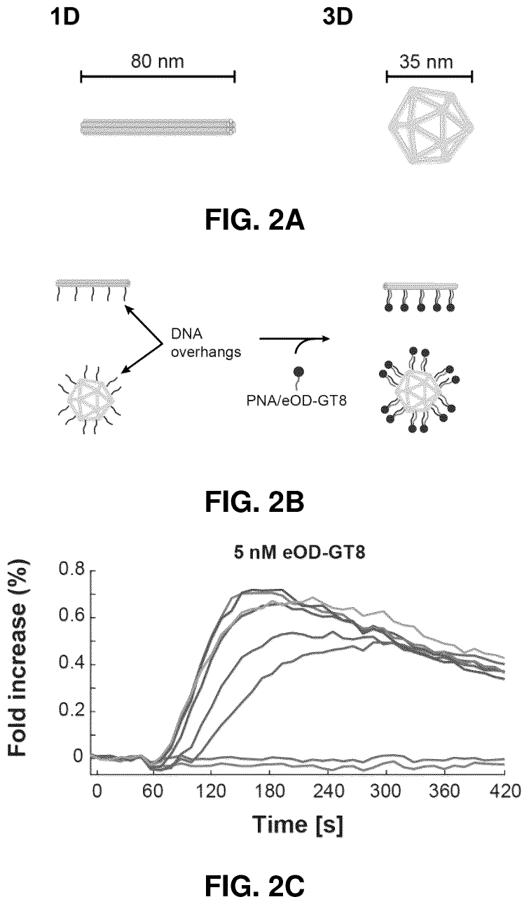

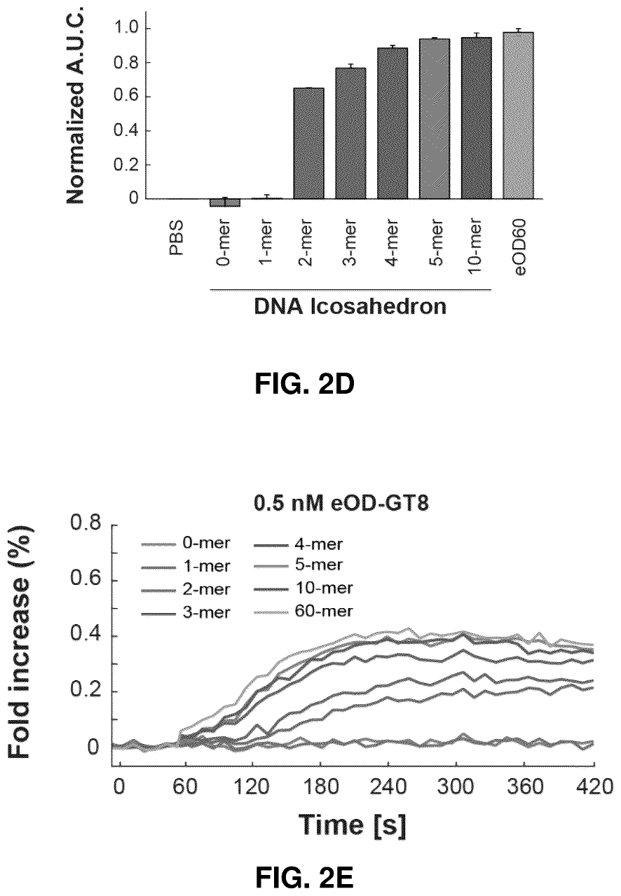

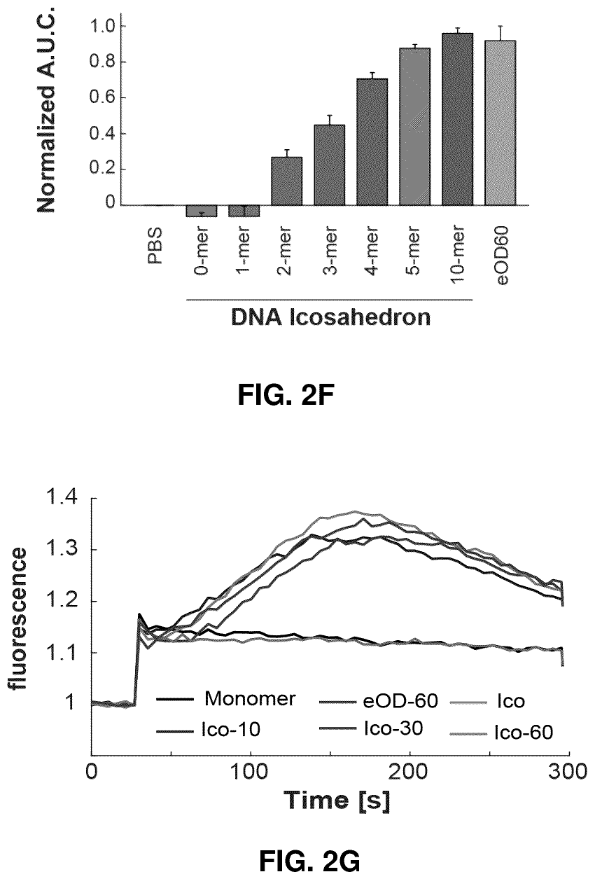

The shape, monodispersity, and antigen modification of DNA-NPs were first confirmed. Negative stain transmission electron microscopy images and agarose gel electrophoresis of both 6-helix bundle structures as well as icosahedra structures (FIG. 2A) demonstrated the monodispersity of nanoparticles, their accurate folding, and their structural rigidity, consistent with previous scaffolded DNA nanostructures designed and prepared with these methods (Venziano, et al., Science, 352(6293):1534 (2016)). TEM images showed high folding yield and monodisperse nanoparticles.

Outwardly-facing DNA overhangs were engineered at the 3′ end of selected DNA nanostructure staple strands, allowing for addressable modification of DNA nanostructures through base-pairing interactions (FIGS. 1D, 1E, and 2B). A monomeric version of eOD-GT8 expressed with an N-terminal cysteine was modified with a complementary PNA strand that was functionalized with a thiol-reactive maleimide group. The purified PNA/eOD-GT8 conjugate was then added to folded and purified nanoparticles and allowed to hybridize fully before final purification and use. Gel electrophoresis assay, tryptophan fluorescence assay, and fluorimetry demonstrated the efficient complexation between the different DNA nanoparticles and PNA/eOD-GT8 (Table 2).

TABLE 2
Percentage of antigen modification

| | | Concen- | Number | Concentration | |
|---|---|---|---|---|---|
| | Fluorescence | tration | of | antigen | Coverage |
| DNA-NPs | intensity | DNA | antigens | (nM) | (%) |
| 30 mer | 2131936.4 | 54.0 | 30 | 1455.3 | 89.8 |
| 60 mer | 3161241.1 | 46.5 | 60 | 2158.0 | 77.3 |
| 5 mer | 1883754.7 | 250.1 | 5 | 1285.9 | 102.8 |
| 1 mer | 67475.9 | 43.0 | 1 | 46.1 | 107.1 |
| 6HB 5 mer | 1045677.6 | 136.0 | 5 | 713.8 | 105.0 |
| 6HB 2 mer | 297036.7 | 107.0 | 2 | 202.8 | 94.8 |
| 6HB 1 mer | 359982.8 | 238.0 | 1 | 245.7 | 103.3 |

The effect of antigen valency was evaluated by comparing icosahedral DNA NP bearing 0, 1, 2, 3, 4, 5, or 10 eOD-GT8 ([0-10]-mer) with the 60-mer protein nanoparticle (eOD60) while keeping the total amount of eOD-GT8 constant, and observing calcium release in a human Ramos cell line stably expressing the germline VRCO1 IgM. While DNA NP modified with a single eOD-GT8, and unmodified DNA NP did not stimulate cells, increasing antigen valency yielded robust cellular responses from 2-mer DNA NP to 10-mer DNA NP (FIGS. 2C-2F). This dependence on valency was even more apparent at a lower concentration of eOD-GT8 (0.5 nM). It was observed that 5-mer and 10-mer eOD-DNA NP resulted in calcium release nearly identical to eOD60 protein nanoparticle, which has a much higher antigen valency. Moreover, using a higher valency on the DNA icosahedron (30- and 60-mer) did not lead to an increase of the cellular response (FIG. 2G). These results indicate that increasing the number of antibody binding sites per nanoparticle above 5 does not lead to stronger cellular responses, and that many antigen sites on eOD60 are dispensable for B cell activation by high affinity antigen.

### Example 2: Increasing Inter-Antigen Distance on a Rigid Scaffold Initially Increases B Cell Receptor Response

### Methods and Materials

Preparation of DNA nanostructures including folding and purification, and attachment of the DNA nanostructures to PNA-antigen conjugates was performed as described in Example 1.

### Antigens and Cell Lines

Ramos B cells expressing VRC01 germline B cell receptor were provided by Daniel Lingwood (Ragon Institute).

### B Cell Calcium Flux Assay

Ramos B Cells at a concentration of 10 million cells/mL were incubated with 10 M Fluo-4 AM (Thermo Fisher) for 30 minutes at 37C. After washing once, flux assays were performed on a Teican plate reader at 37C on a 96 well microplate. 160 μL of Fluo-4 labeled Ramos cells at 2 million cells/mL was added to each well. A baseline fluorescence was then recorded for 1 minute, and 40 μL of nanoparticles were added to the cells for a final concentration of 5 nM of antigen, unless otherwise stated.

### Results

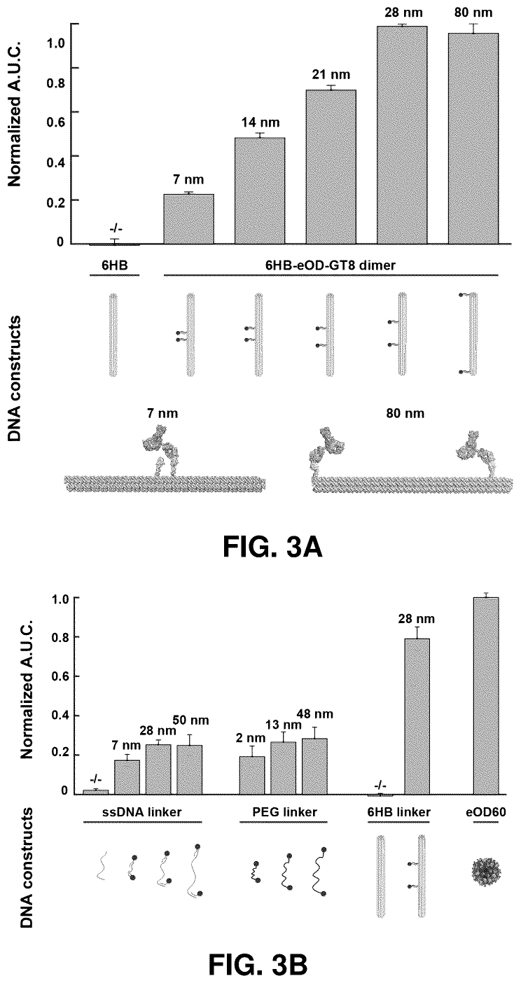

Previous results have indicated that inter-antigen distances impact receptor signaling for both the IgE Fc receptor (Sil, et al., ACS Chem. Biol., 2(10):674-84 (2007)) and the T cell antigen receptor (Cochran, et al., J. Biol. Chem., 276(30):28068-28074 (2001)), and that the B cell receptor oligomeric organization impacts receptor activation (Yang, et al., Nature, 467(7314):465-469 (2010)). The impact of spacing between epitopes on B cell receptor engagement was explored by systematically varying inter-antigen distance on a rigid and linear 6 helical DNA NP rods templating two eOD-GT8 per nanoparticle (FIG. 3A). For distances less than 28 nm, calcium responses were systematically lowered as inter-antigen distance decreased. A decrease in cellular response with longer inter-antigen distances up to 80 nm, the full length of the nanoparticle rod, was not observed even though this distance is well beyond the size where two separate immunoglobulin Iga/0 pairs could be interacting (FIG. 3A; bottom inset).

To explore whether the observed B cell response was reliant on DNA NP rigidity, eOD-GT8 dimers were created on flexible polymeric ssDNA linkers and PEG linkers having comparable extended length as the DNA NP 6-helix bundle structure (FIG. 3B, Table 3).

TABLE 3
Linker characteristics

| | | | Number | | |
|---|---|---|---|---|---|
| | | | of units | Linker | Flory |
| Linker | Linker | MW | (Bases or | length | radius |
| material | name | (Da) | PEG units) | (nm) | (nm) |
| ssDNA | ssDNA5 | 8930.9 | 5 | 3.2 | N/A |
| ssDNA | ssDNA12 | 11123.3 | 12 | 7.6 | N/A |
| ssDNA | ssDNA24 | 14881.8 | 24 | 15 | N/A |
| ssDNA | ssDNA35 | 18210.9 | 35 | 22 | N/A |
| ssDNA | ssDNA47 | 21916.2 | 47 | 30 | N/A |
| ssDNA | ssDNA83 | 30767.9 | 83 | 52 | N/A |
| PEG | Bis-Mal-PEG-2 | 308.3 | 2 | 0.6 | 0.5 |
| PEG | Bis-Mal-PEG-3 | 352.3 | 3 | 0.8 | 0.5 |
| PEG | Bis-Mal-PEG-4 | 2000 | 45 | 12.6 | 2.8 |
| PEG | Bis-Mal-PEG-5 | 3500 | 80 | 22.4 | 3.9 |
| PEG | Bis-Mal-PEG-6 | 5000 | 114 | 31.9 | 4.8 |
| PEG | Bis-Mal-PEG-7 | 7500 | 170 | 47.7 | 6.1 |

Flexible polymer dimers gave a drastically reduced cellular response compared to the rigid DNA NP dimers, indicating the importance of structural form and rigidity during B cell receptor responses to antigen.

### Example 3: Clustering of Antigens on One Face of an Icosahedron does not Improve B Cell Receptor Responses Compared to Positioning on a Linear Rod

### Methods and Materials

Preparation of DNA nanostructures including folding and purification, and attachment of the DNA nanostructures to PNA-antigen conjugates was performed as described in Example 1.

### Antigens and Cell Lines

Ramos B cells expressing VRCO1 germline B cell receptor were provided by Daniel Lingwood (Ragon Institute).

### B Cell Calcium Flux Assay

Ramos B Cells at a concentration of 10 million cells/mL were incubated with 10 M Fluo-4 AM (Thermo Fisher) for 30 minutes at 37C. After washing once, flux assays were performed on a Teican plate reader at 37C on a 96 well microplate. 160 μL of Fluo-4 labeled Ramos cells at 2 million cells/mL was added to each well. A baseline fluorescence was then recorded for 1 minute, and 40 μL of nanoparticles were added to the cells for a final concentration of 5 nM of antigen, unless otherwise stated.

### Results

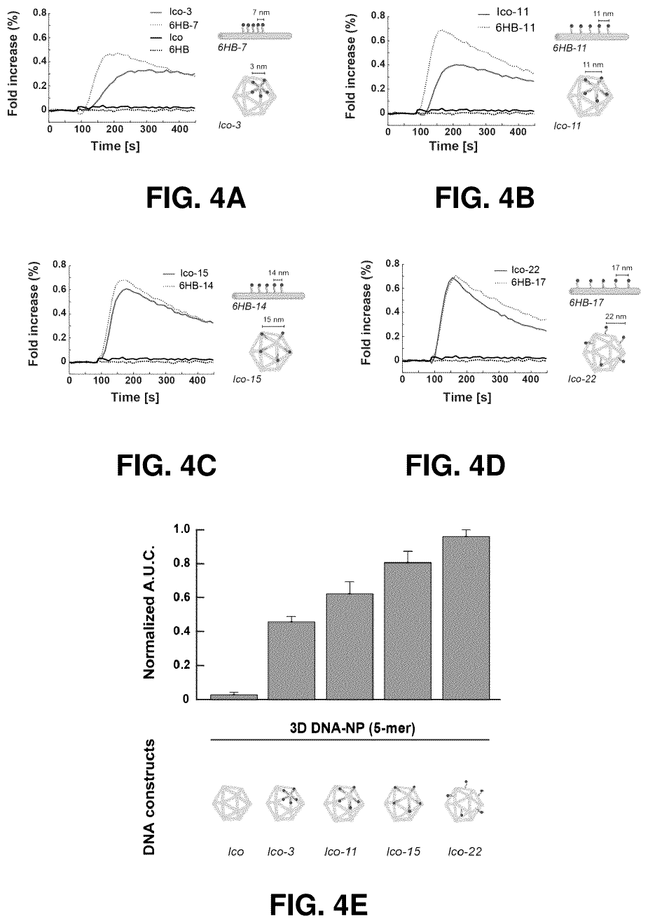

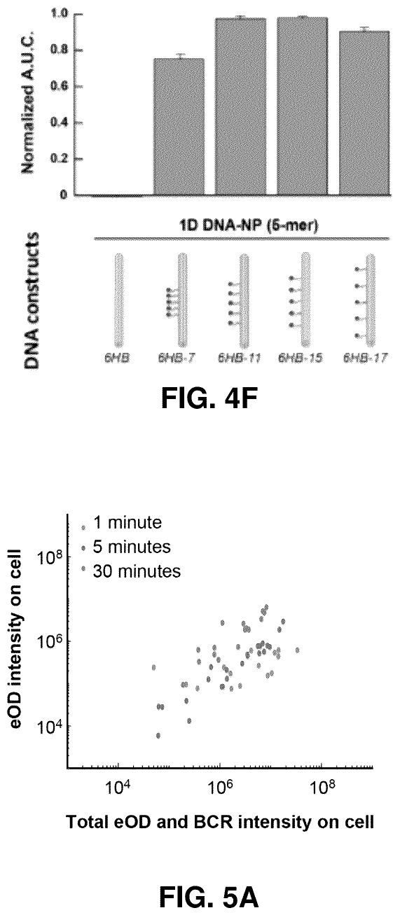

The results described in Example 2 indicate that tight clustering of antigen may limit B cell receptor responses due to close inter-antigen distances, in contrast to a model where tight clustering of antigen leads to maximal B cell receptor responses by facilitating inter-BCR cooperativity. To further test this, a linear placement of five eOD-GT8 antigens on a 6HB DNA NP was compared to a clustered placement of five eOD-GT8 antigens around one face of the icosahedron DNA NP for different inter-antigen distances between −5 nm and 22 nm (FIGS. 4A and B). Notably, the average distance between all antigen sites on the icosahedron 5-mer is -2/3 that of the 6HB linear rod 5-mer. It was observed that for the large inter-antigen distances, 22 nm and 15 nm, clustering on both linear and planar structures led to an equivalent cellular response (FIGS. 4C-4D). However, when inter-antigen distance is small, such as 11 nm and 3 nm, linear placement of eOD-GT8 antigens yielded a greater response than a clustered planar placement of antigens (FIGS. 4A-4B). Further, decreasing the distance between antigens in both the planar and the linear presentation yielded a decreased cellular response, consistent with previous results from dimer DNA NP structures (FIGS. 4E-4F).

### Example 4: Immunogen Presenting Nanoparticles Facilitate Specific Binding and Activation of the B Cell Receptor

### Methods and Materials

Preparation of DNA nanostructures including folding and purification, and attachment of the DNA nanostructures to PNA-antigen conjugates was performed as described in Example 1.

### Antigens and Cell Lines

Ramos B cells expressing VRC01 germline B cell receptor were provided by Daniel Lingwood (Ragon Institute).

Antigen Conjugation with AF647 Dye

The eOD-PNA conjugate was modified with the fluorescent label AlexaFluor 647-NHS (AF647). The conjugate was incubated with 5 molar equivalents of AF647-NHS in 10 mM sodium bicarbonate buffer for 2 hours at room temperature. Unreacted dye was removed using centrifugal filtration (10 kDa MWCO).

### B Cell Imaging: Sample Preparation for Confocal Microscopy

Ramos cells were labeled on ice at a concentration of 5 million/mL and protected from light for 30 minutes in Hank's Buffered Sterile Saline (HBSS) with 20 μg/mL human anti IgM f(Ab)₁ fragment (Jackson 109-007-043) conjugated to Janelia Fluor 549. Cells were spun down and resuspended in warm HBSS at a concentration of 2 million/mL. Antigens were added to a final concentration of 5 nM by adding 50 μL antigen solution to a volume of cells between 175 μL and 400 μL, and cells were kept at 37C by incubation in a thermal bead bath. At timepoints following the addition of antigen, 100 μL of cells were removed and placed into 200 μL of 6% warm PFA solution and allowed to fix for 10 minutes at 37C. Following fixation, fixed cells were diluted in 4.5 mL HBSS and centrifuged at 600g for 5 minutes. Cells were then labeled for 5 hours at 4C in 50 μL HBSS with 5 mg/mL bovine serum albumin (BSA, Sigma) with 10 μg/mL wheat-germ agglutinin (WGA) conjugated to Alexa 488 (ThermoFisher W11261), and a 1:50 dilution of Phalloidin conjugated to Alexa 405 (material from Sudha). Cells were diluted into 4.5 mL HBSS and centrifuged at 600g for 5 minutes, and resuspended in 4.5 mL HBSS and centrifuged again to wash before being resuspended in 100 μL HBSS. Cells were then plated onto LabTech II 8-well glass bottom chambers modified with 0.1% Poly-L-Lysine (PLL, Sigma P8920) and allowed to adhere for at least 4 hours at 4C before performing confocal microscopy.

### Confocal Microscopy Imaging

Confocal microscopy was performed on a Zeiss AxioVert 200 μM inverted microscope stand with Yokogawa CSU-22 spinning disk confocal scan head with Andor Borealis multi point confocal system. Probes were excited by 4 laser lines in the Andor/Spectral applied Research Integrated Laser Engine: 405 nm 100 mW OPSL, 488 nm 150 mW OPSL, 561 nm 100 mW OPSL, and 642 nm 110 mW OPSL. Multipass dichroic mirror 405/488/568/647 and emission filters 450/50 nm, 525/50 nm, 605/70 nm, and 700/75 nm were used for each emission channel, respectively. Sample was imaged through a 63× oil Plan Apochromat objective with an effective pixel size of 0.092 m/pixel. Images were captured through a Hamamatsu Orca-ER cooled CCD, and instrumentation was controlled through MetaMorph software. For each image, 9 z-planes having separation of 1.5 m were acquired between the top and bottom of the cell, and approximately 10 fields of view were acquired for each sample.

### Image Analysis

16-bit images were read into MATLAB and converted to double precision. For each field of view, a maximum intensity projection (MIP) was calculated for the phalloidin channel. This was then binarized using adaptive thresholding, cleaned of stray pixels, and then morphological opening and closing was performed. Holes within this binarization were then filled, and discreet objects within this binarization were labeled as individual cells. For each cell in a field of view, z-planes were binarized as above using the phalloidin channel, and these z-plane binarizations were restricted to the limit of the MIP binarization for each cell. The convex hull of this z-plane binarization was used to estimate the extent of the cell, and the cell surface was estimated by selecting the perimeter of the z-plane binarization and dilating this 25 times in a 4-connected neighborhood and subsequent restriction by the undilated cell extent binarization. Total probes intensity and surface probes intensity of cells was calculated through summation through all z-stacks after logical indexing of the background-subtracted raw z-plane images, where background was estimated to be a constant through all z-planes and channels. Pixel-based correlation was performed through pairwise linear correlation of pixel values between channels following logical indexing. Average intensity values shown are an average over cells, and errorbars shown are the standard error of the mean, given by the standard deviation divided by sqrt(N_{cells}). Internalized fraction of probe intensity for a single cell is given by (total cell intensity -surface cell intensity)/total cell intensity.

### Results

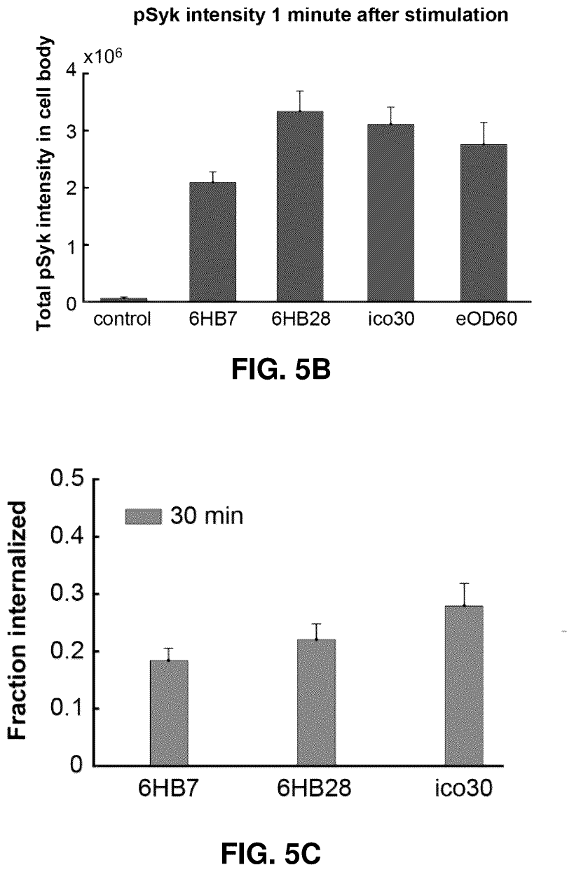

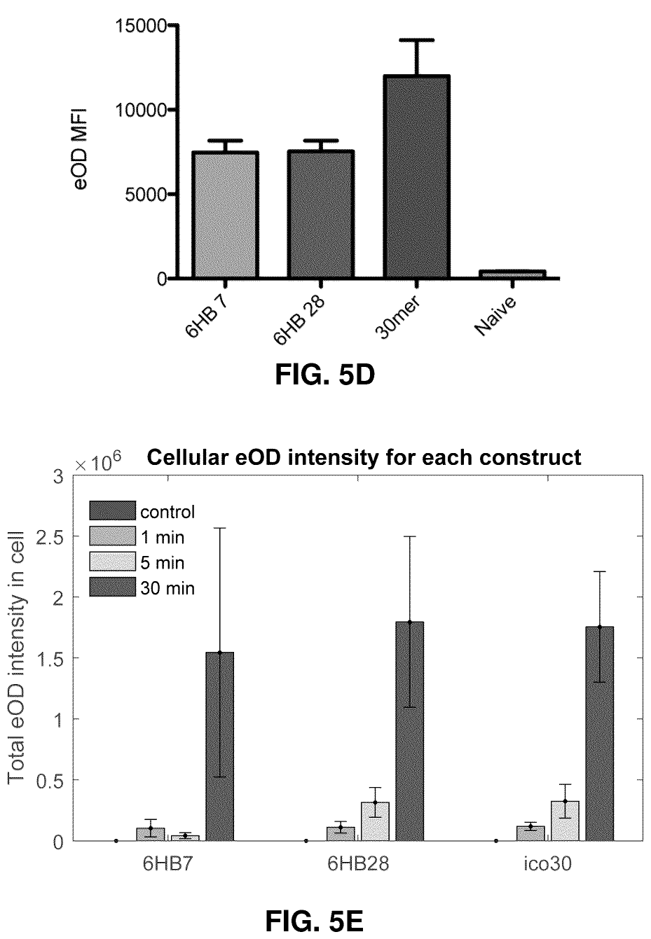

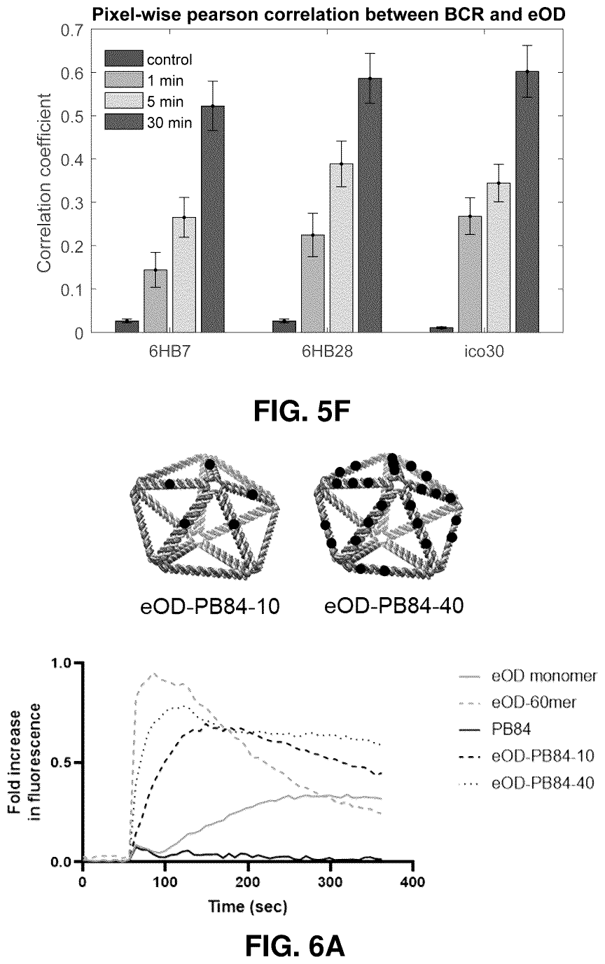

Three eOD-GT8 DNA NP constructs (Icosahedron-30 mer eOD-GT8, six-helix bundle dimer with 28 nm spacing, and six-helix bundle dimer with 7 nm spacing) were chosen to examine in more detail through confocal microscopy using a fluorescent eOD that was labeled with Alexa Fluor 647. DNA NP eOD constructs bound to Ramos cells in an approximately equal fashion and were correlated in space with the B cell receptor on cell surfaces and were co-internalized at long times (FIGS. 5D-5F). eOD binding was highly correlated with VRCO1 IgM expression, and cells lacking IgM expression failed to bind eOD (FIG. 5A). Antibody staining of phosphorylated Syk kinase (pSyk) revealed a sharp increase in pSyk phosphorylation after 1 minute of DNA NP or eOD60 addition for all DNA NP constructs (FIG. 5B). Dimer eOD separated by short distances (6HB7, 7 nm dimer) resulted in significantly less pSyk binding as compared to the dimer eOD separated by long distance (6HB28, 28 nm dimer) and the icosahedron bearing 30 eOD (ico30) (FIG. 5B). The internalization of eOD was also examined by use of the phalloidin stain to estimate cellular boundary. Compared to the 6HB7, it was observed that eOD internalization improved for longer inter-antigen distances and the icosahedron 30-mer (FIG. 5C).

The disclosed compositions include materials, compounds, and components that can be used for the disclosed methods. Various exemplary combinations, subsets, interactions, groups, etc. of these materials are described in more above. However, it will be appreciated that each of the other various individual and collective combinations and permutations of these compounds that are not described in detail are nonetheless specifically contemplated and disclosed herein. For example, if one or more nucleic acid nanostructures are described and discussed and a number of substitutions of one or more of the structural or sequence parameters are discussed, each and every combination and permutation of the structural or sequence parameters possible are specifically contemplated unless specifically indicated to the contrary.

These concepts apply to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### Example 5: Immunogen Presented by Pentagonal Bipyramidal Architectures Efficiently Activated B Cell In Vitro

### Methods and Materials

Experimental procedures are identical to those described under Example 1 with the following additions and modifications.

### Chemicals and Kits

Pluronic F-127 (10% (w/v) solution) was obtained from Sigma Aldrich. Corning Spin-X UF-0.5 ml centrifugal filters (MWCO 100 kDa) were obtained from Sigma Aldrich.

ssDNA Scaffold Synthesis

Custom circular ssDNA scaffolds of lengths matching the corresponding nucleic acid nanostructure architectures were produced in a phage-based approach as previously described (see Shepherd, Sci Rep, 9(1):61212 (2019)). Briefly, SS320 (Lucigen) E. coli were fist transformed with the M13cp helper plasmid and subsequently with the ssDNA encoding phagemids phPB84 and phI52. For milligram-scale production of synthetic miniphage, a Stedium Sartorius fermenter was used for growing 5 μL of culture. For 1 μL cultures production was conducted in regular culture flasks and the following steps were carried out in analogy to the 5 μL culture. An overnight culture of phage-producing colonies, as determined by PCR, gel visualization, and sequencing results, was grown in 2 x YT supplemented with 100 μg/mL Ampicillin and 15 μg/mL of chloramphenicol and 5 μg/mL of tetracycline and diluted to O.D. 600 of 0.05 for inoculating 5 μL of media. The growth media for the batch fermentation was also 2 x YT supplemented with 100 μg/mL Ampicillin and g/mL of chloramphenicol and 5 μg/mL of tetracycline. Oxygen and pH were monitored throughout the growth, and the pH was maintained at 7.0 with phosphoric acid and ammonium hydroxide, with a constant agitation of 400 RPM. At 8 h, the phage-containing bacteria culture was harvested and processed. For milligram-scale purification of ssDNA, 900 mL batches of liquid culture bacteria were processed as follows. Bacteria were pelleted by centrifuging twice at 4,000×g for 20 min, followed by 0.45 m cellulose acetate filtration. Phage from clarified media were precipitated by adding 6% w/v of polyethylene glycol-8000 (PEG-8000) and 3% w/v of NaCl and stirring continuously at 4° C. for 1 h. Precipitated phage were collected by centrifuging at 12,000×g for 1 h, and the PEG-8000 supernatant was removed completely, and pellet was resuspended in 30 mL of 10 mM Tris-HCl pH 8.0, 1 mM Ethylenediaminetetraacetic acid (EDTA) buffer (TE buffer). The phage was then processed using an EndoFree Maxiprep (Qiagen, Germany) column-based purification, following the manufacturer's protocol with two adjustments. First, proteinase K (20 μg/mL final) was added to EndoFree Buffer P1 and incubated at 37° C. for 1 h before addition of EndoFree Buffer P2 and incubation at 70° C. for 10 min. The lysed phage was returned to room temperature before proceeding. Second, after removal of endotoxins, 0.2 v/v of 100% ethanol was added to the clarified sample, before applying to the EndoFree Maxiprep column to increase ssDNA binding. All other steps remained the same, and the circular ssDNA was eluted in 1 mL of endotoxin-free TE buffer. The amount of collected DNA was judged by absorbance at A280, and the purity was judged by running on a 1% agarose gel in lx TAE stained with ethidium bromide.

### DNA Origami Nanostructure Folding

In addition to the protocol described under Example 1, guard staples complementary to the overhangs sequence were added prior to thermal annealing at 5-fold excess to ensure monodispersed assembly of the DNA origami nanostructures.

### Purification of Functionalized DNA Origami Nanostructures

Antigen-functionalized DNA origami nanostructures were purified using Spin-X UF-0.5 ml centrifugal filters (MWCO 100 kDa). Prior to use for purification, the filter membrane was passivated with 5% (w/v) Pluronic F-127 for 30 min at room temperature and subsequently washed three times with PBS at pH 7.4.

### Characterization of Functionalized DNA Origami Nanostructures

In addition to the methods described under Example 1 to validate purity, structural integrity and immunogen copy number, functionalized DNA origami nanostructures were additionally analyzed by dynamic light scattering and atomic force microscopy.

### Results

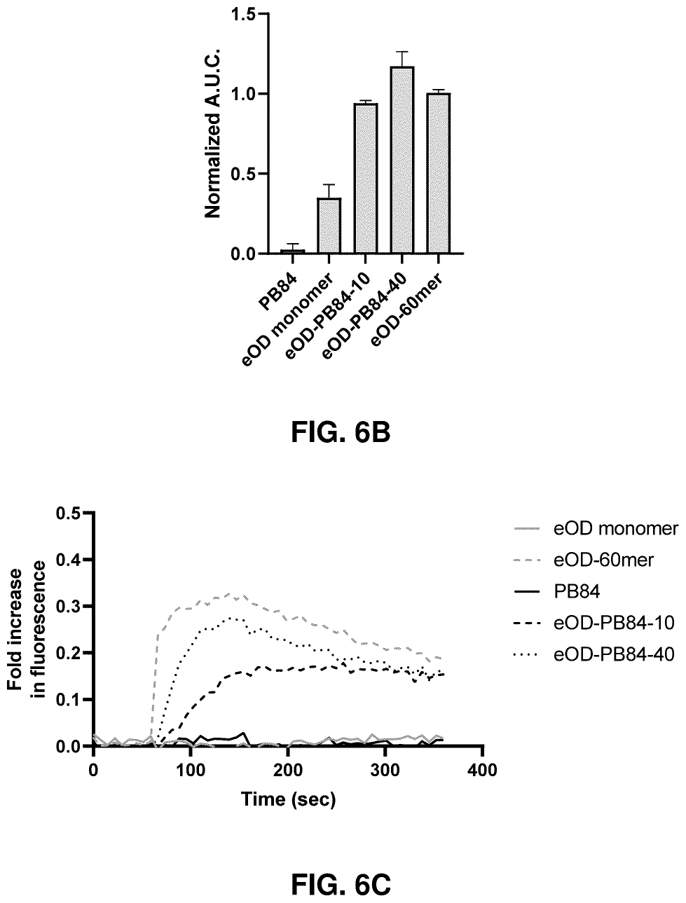

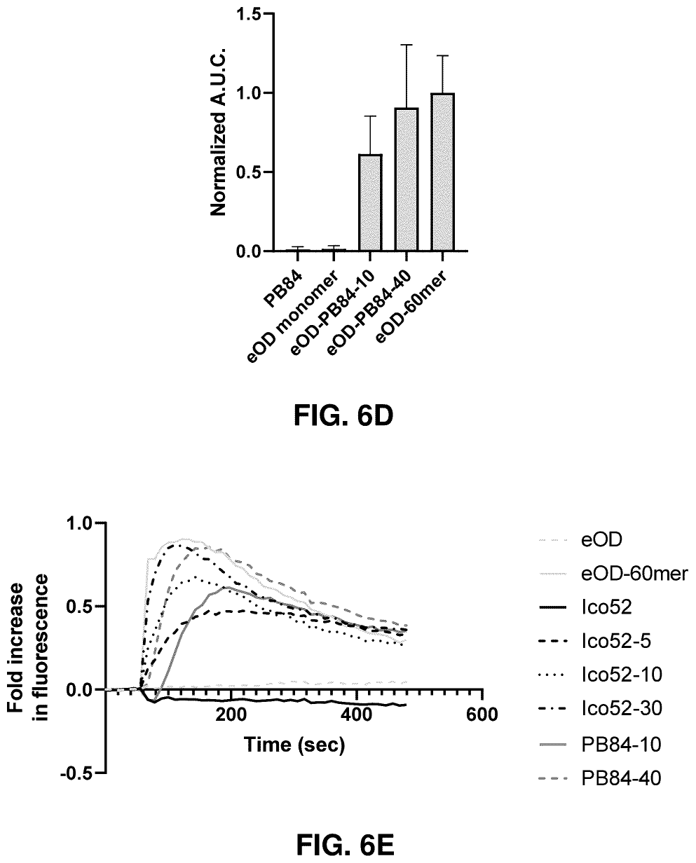

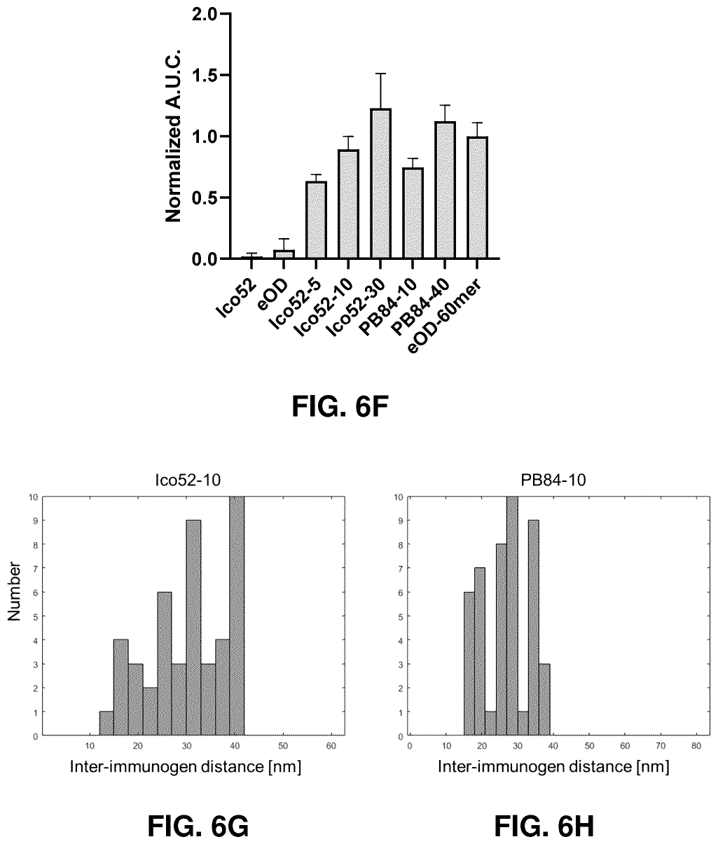

Purity, structural integrity, immunogen copy number and monodispersity were characterized for both the icosahedron and the pentagonal bipyramid as described under Example 1. Initially, the activation of human Ramos B cells by a pentagonal bipyramid with 84 bp edge length carrying 10 or 40 copies of eOD-GT8 was assayed in a calcium flux assay. Activation levels were compared to the eOD-GT8-60mer protein nanoparticle at total immunogen concentrations of 5 nM (FIGS. 6A and B) and 1 nM (FIGS. 6C and D). As observed for the icosahedral architecture, the pentagonal bipyramid efficiently activated B cell in vitro. At 5 nM immunogen concentration, activation levels saturate for both 10 and 40 copies of immunogen and are comparable to or higher than the eOD-GT8-60mer protein nanoparticle reference. At lower immunogen concentrations, the activation level for 10 copies of immunogen is decreased, while the 40 copy DNA nanostructure activates B cells comparably to the eOD-GT8-60mer protein nanoparticle.

The direct comparison between the pentagonal bipyramidal architecture with the icosahedron at total immunogen concentrations of 2 nM revealed that the latter activates B cells more efficiently in vitro (FIGS. 6E and F). Activation at both low (10 copies) and high (30 and 40 copies) valency are increased for the icosahedral architecture. While results for nanostructures with different copy numbers are difficult to compare, in part due to different nanostructure concentrations in the experiment, an assessment of inter-immunogen distance histograms for 10 copy nanostructures for the icosahedron and pentagonal bipyramid is helpful to interpret these observations. The distance histograms reveal that the distribution is skewed towards larger values for both minimal and maximal distances in case of the icosahedron (FIGS. 6G and 6H). This correlates with stronger activation and is in accordance with the observations made in Examples 1 to 3.

The results indicate that icosahedral architectures are superior to pentagonal bipyramidal architectures for immunogen presentation.

### Example 6: Peptide Immunogen Presentation on Nucleic Acid Nanostructures is Comparable to Liposomal Formulations

### Methods and Materials

Experimental procedures are identical to those described under Examples 1 and 6 with the following additions and modifications.

### Chemicals and Kits

Peptide-PNA-Alexa Fluor 647 conjugates were obtained from PNA Bio. Peptides for conjugation to liposomes were obtained from GenScript.

### Liposome Synthesis

Lipids 1,2-dioleoyl-sn-glycero-3-phosphocholine (DOPC), 1,2-dimyristoyl-sn-glycero-3-phosphocholine (DMPC), 1,2-dioleoyl-sn-glycero-3-phospho-(1′-rac-glycerol) (DOPG), 1,2-distearoyl-sn-glycero-3-phosphoethanolamine-N-[maleimide(polyethylene glycol)-2000](PEG-DSPE-mal), and monophosphoryl lipid A (MPLA) were obtained from Avanti Polar Lipids. Peptide-conjugated liposomes were prepared as previously described with some modifications (see Tokatlian et al., Enhancing Humoral Responses Against HIV Envelope Trimers via Nanoparticle Delivery with Stabilized Synthetic Liposomes. Scientific Reports 8, (2018)). Unilamellar liposomes formed of DOPC:DMPC:DOPG:PEG-DPSE-mal:MPLA lipids in a 37.8:37.8:18.9:5:0.5 molar ratio were synthesized by lipid film rehydration and membrane extrusion using a 100 nm membrane, followed by post-synthesis binding of cysteine-terminated peptides for 2 hours at room temperature in PBS. Unconjugated peptide was removed via dialysis using 10 kDa cutoff membranes in 1 μL PBS baths with three bath exchanges over 36 hours. Liposome size and uniformity was assessed via dynamic light scattering, and peptide conjugation was quantified via tryptophan fluorescence.

### Antigens and Cell Lines

Peptide sequences were chosen from a previously reported set of H-2K^{k} MHC class I-derived molecules which can induce HIV antigen-specific B cell responses (see Kouskoff, et al, Journal of Experimental Medicine, 188(8):1453-64 (1998)). In particular, one high-affinity peptide, p31, and one intermediate affinity peptide, p5, were chosen. Peptides were C-terminally conjugated to the PNA sequence using an amide-DEG linker. The PNA sequence itself bears a C-terminal AF647 label that enables the validation of copy number on the DNA nanostructures. Peptide-PNA-Alexa Fluor 647 conjugates were dissolved in DMF:H₂O such that the final DMF concentration during hybridization of the PNA to the overhang staple on the nucleic acid nanostructure was less than 5%.

Primary 3-83 splenocytes were obtained from NOD.D2(B10)-Tg(Igh2^{k}3-83)1Nemz/Dvs mice (The Jackson Laboratory). This mouse strain produces only a single B cell receptor with known specificity for a series of previously defined peptide antigens (see Kouskoff, et al, Journal of Experimental Medicine, 188(8):1453-64 (1998)).

### B Cell Calcium Flux Assay

Primary 3-83 splenocytes at a concentration of 10 million cells/mL were incubated with 10 μM Fluo-4 AM (Thermo Fisher) for 30 minutes at 37C. After washing once, flux assays were performed on a Teican plate reader at 37C on a 96 well microplate. 160 μL of Fluo-4 labeled 3-83 cells at 2 million cells/mL was added to each well. A baseline fluorescence was then recorded for 1 minute, and 40 μL of nanoparticles were added to the cells for a final concentration of 1 nM of antigen, unless otherwise stated.

### Results

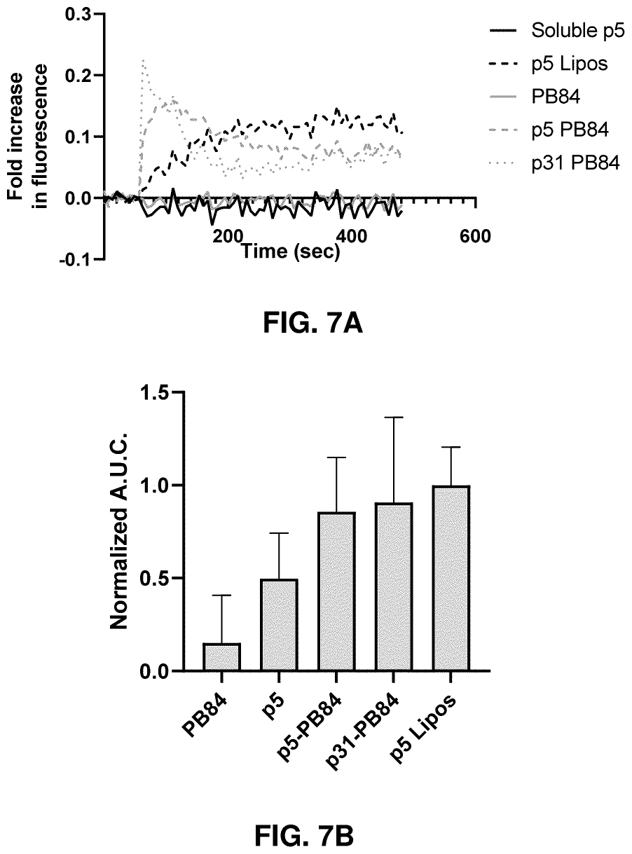

Purity, structural integrity, immunogen copy number and monodispersity were characterized for both the icosahedron and the pentagonal bipyramid as described under Examples 1 and 5. Both the pentagonal bipyramid functionalized with 45 copies of the high-affinity peptide p31 and the intermediate-affinity peptide p5 efficiently activated 3-83 cells in vitro compared to the liposome reference. Notably, the activation kinetics are faster for the nucleic acid nanostructures and resemble those observed for the presentation of eOD-GT8 antigens on nucleic acid and protein nanostructures, corroborating the proposed favorable effect of rigid presentation. Furthermore, the high-affinity peptide p31 displayed stronger activation compared to p5. Overall, these results demonstrate the suitability of the DNA origami platform for presentation of different classes of antigens including both proteins and peptides. See, e.g., FIGS. 7A and 7B.

### Example 7: MBL and C1q Opsonization Play a Role in VLP Immunogenicity In Vivo

To explore the roles of MBL and C1q in DNA-VLP trafficking and immunogenicity, wild-type C57BL/6 mice or MBL- and C1q-deficient mice were immunized with either I42-60x-eOD-PADRE or eOD-60mer (FIG. 14A). Serum collected prior to immunization was used to confirm by ELISA the absence of MBL or C1q binding in the respective knockout 5 groups (FIGS. 14B-D). Two weeks post-immunization, anti-eOD serum titers were measured, and germinal center (GC) B cell responses in the inguinal lymph nodes were quantified by flow cytometry. The MBL KO group showed significantly reduced serum antibody titers for both eOD-60mer and DNA-VLPs, accompanied by a modest decrease in GC B cell counts. In contrast, C1q deficiency did not markedly affect serum antibody responses, although GC B cell numbers expanded by DNA-VLPs were roughly two-fold higher than in wild-type mice.

These observations demonstrate that MBL positively contributes to DNA-VLP delivery into follicles, whereas the impact of C1q appears more nuanced, potentially involving pathway redundancy or dual roles in complement activation versus clearance.

### Example 8: DNA Origami Vaccines Program Antigen-Focused Germinal Centers

As an alternative to protein-based scaffolds, virus-like particles (VLPs) formed by the programmed assembly of thymus-independent (TI) DNA origami (DNAVLPs) enable precise antigen display and enhance antigen-specific antibody titers while avoiding anti-scaffold B cell responses. DNA origami has been shown to be minimally immune-stimulatory, unless specific CpG motifs are included to activate innate Toll-like receptor pathways. Thus, DNA origami may serve as an attractive vaccine scaffold due to its highly programmable geometry and size, quantitative spatial precision for display of biomolecular cargoes, and immunologically inert TI scaffold. Accordingly, it was hypothesized that DNA origami scaffolds could enable a rigorous test of the effects of scaffold-specific competitor B cells on the recruitment and affinity maturation of rare “on-target” precursors in primary GCs. To test the hypothesis, icosahedral DNA-VLPs conjugated with eOD-GT8 were rationally designed as a clinically relevant test immunogen.

### Results

Scaffolding eOD-GT8 on DNA origami virus-like particles SARS-CoV-2 receptor-binding domain multimerized on icosahedral DNA-VLPs (30 antigens/nanoparticle) elicited high serum IgG titers in mice after a prime-boost vaccination that potently neutralized the virus.

To begin exploring how DNA-VLP design impacts earlier stages of the immune response, and particularly the formation of primary germinal center responses that are crucial to effective HIV vaccines, the same DNA-VLP and surface-conjugation was employed with 30 equally spaced copies of the eOD-GT8 antigen to the particles. This wireframe DNA origami nanoparticle was computationally designed using DAEDALUS software and synthesized following established protocols, utilizing a custom ssDNA scaffold folded with 30 DBCO-modified staple oligonucleotides and reacted with eOD-GT8 containing an N-terminal azido linker. Successful DNA-VLP self-assembly was confirmed through agarose gel electrophoresis (AGE) and dynamic light scattering. Post-antigen coupling, the hydrodynamic radius of the nanoparticles was −40 nm, which are annotated as d40_30mer.

The percentage of alkyne groups on the DNA-VLP successfully conjugated with antigen was quantified using a bicinchoninic acid protein assay; functionalization efficiency across independent sample preparations was consistently in the range of 90-100%.

The measured antigen concentrations were used to standardize eOD-GT8 doses across experimental groups. Cryo-electron microscopy and negative stain transmission electron microscopy validated the preservation of the icosahedral geometry of the origami structure post-antigen conjugation. The bioavailability and proper orientation of the antigen were further confirmed through binding assays with murine VRCO1, a broadly neutralizing antibody targeting the CD4 binding site of gpl20 that binds with high affinity to eOD-GT8. VRCO1 antibody successfully bound eOD-GT8 displayed on DNA-VLPs, as evidenced by decreased mobility in gel electrophoresis. As a functional quality control metric for antigen bioactivity, antigen-loaded particles were incubated with Ramos B cells expressing the germline VRCO1 B cell receptor, and measured calcium flux using a calcium-dependent fluorescent dye.

Culture of VRCO1-expressing Ramos B cells with 5 nM eOD-GT8-functionalized DNA-VLPs led to rapid B cell activation as assessed by calcium signaling, while the same dose of soluble eOD-GT8 monomer failed to trigger calcium release.
d40_30mer DNA-VLPs enhance serum antibody titers in prime-boost immunization but are weak priming immunogens

Based on the results from in vitro B cell activation and previous in vivo studies of eOD-GT8 protein nanoparticles, it was hypothesized that d40_30mer would induce higher antibody responses in mice compared to eOD-GT8 monomer due to its multivalency. For immunization studies, we employed a saponin-MPLA nanoparticle adjuvant (SMNP), a potent ISCOMs-like adjuvant. Mixing DNA-VLPs with SMNP did not affect the stability of the DNA-VLPs. C57BL/6 mice were primed and boosted with equimolar doses of eOD-GT8 monomer or d40_30mer co-administered with SMNP, and antigen-specific serum IgG titers were monitored over time. Both eOD-GT8 monomer control and DNA-VLPs resulted in weak responses post-prime, but following boosting the DNA-VLP elicited endpoint antibody titers -1 log higher than the eOD-GT8 monomer. Antibody responses were additionally assessed against the bare DNA-VLP, and consistent with prior studies, no elevation in anti-origami IgG or anti-dsDNA IgG was observed after prime or boost. IgM responses to dsDNA were also measured and no response above the nonspecific background triggered by protein eOD-GT8/SMNP immunization was detected in the complete absence of DNA.

To gain insight into the early stages of the response to monomer vs. DNA-VLP immunization, germinal center responses in draining lymph nodes were evaluated at two weeks after a single immunization by flow cytometry. Unexpectedly, immunization with d40_30mer did not increase the number of GC B cells (B220+/CD381o GL7hi) or follicular helper T cells (CD4+/CXCR5hi PDlhi) relative to eOD-GT8 monomer vaccination. The frequency and counts of antigen-specific GC B cells, which were identified by fluorescent eOD-tetramer staining, were also not enriched and remained low. i.e., at below 1%. Alternative adjuvants including alum, AddaVax, ASOlb, and CpG ODN 1826 also did not enhance GC B cell responses elicited by the DNA-VLPs.

A primary concern for DNA origami-based nanoparticles in vivo is whether they remain stable over a sufficient time period in the presence of DNases present in tissues. To evaluate whether the priming efficiency of DNA-VLPs was limited by endonuclease-mediated degradation, vaccine responses in DNase I-deficient mice were assessed. Serum stability assays verified that d40_30mer remained intact in DNase I KO serum for longer than seven days. Further, it was validated that mutant DNase I KO mice established robust GCs in response to protein vaccination. To determine if DNAse-mediated particle degradation was a limiting factor in vivo, WT or DNase I KO mice were primed with d40_30mer and analyzed germinal centers after two weeks. Surprisingly, total GC size, Tfh responses, and antigen-specific GC B cells formed by d40_30mer in DNase I KO animals were low and identical to responses in WT littermates.

Together, the data suggested that the d40_30mer nanoparticle design may provide sufficient avidity to expand antibody-producing cells upon boosting, but the nanoparticle design, irrespective of enzymatic stability, is not optimal for supporting primary GC responses.

d40_30Mer DNA-VLPs have Restricted Access to Follicular Dendritic Cells

To understand why the d40_30mer DNA-VLP poorly primed GC responses, the physiological barriers limiting antigen delivery to B cells in vivo were considered. Antigen arriving at draining lymph nodes during primary immunization is often quickly cleared by lymph flow, protease activity in the tissue, or by endocytosis and degradation by lymphocytes. Alternatively, antibody- or complement-opsonized antigen can be captured by follicular dendritic cells (FDCs) and presented to B cells in germinal centers for prolonged periods to support the GC response. Antigen biodistribution in draining lymph nodes (dLNs) was analyzed following immunization, comparing eOD-GT8 monomer and d40_30mer DNA-VLPs with the protein nanoparticle eOD-GT8 60mer (hereafter, p60mer), which was previously shown to become decorated with complement in vivo via the lectin pathway, leading to rapid robust accumulation on FDCs (which express high levels of complement receptor). To better understand the in vivo fate of DNA-VLPs versus protein immunogens, mice were immunized with fluorescently tagged antigens and collected inguinal dLNs two hours later for cryo-sectioning and immunofluorescence staining.

Early after immunization, eOD-GT8 monomer was primarily detected in central non-follicular areas of dLNs, largely colocalizing with F4/80+medullary sinus macrophages (MSMs). By contrast, eOD-GT8 delivered as d40_30mer was present at substantially higher levels in the draining lymph nodes, and colocalized with CD169+subcapsular sinus macrophages (SSMs) lining the edges of the lymph node and MSMs. The p60mer particle also colocalized with SSMs but was already at this early timepoint accumulating on the dendrites of CD35+FDCs. Quantitative pixel analysis revealed that d40_30mer and p60mer had similar levels of antigen colocalization with SSMs, but only p60mer showed enrichment on FDCs. At later time points such as day 7, p60mer had become highly concentrated on FDCs, whereas follicles in lymph nodes immunized with d40_30mer had no retention of fluorescent antigen. Thus, antigen trafficking and retention of DNA-VLPs was distinct from similarly-sized protein nanoparticles.

It was hypothesized that trapping of d40_30mer in the subcapsular sinus could be due to high expression of DNA-binding scavenger receptors on macrophages and/or lymphatic endothelial cells or inadequate triggering of complement pathways to mediate the capture of the particles by FDCs. To test the first hypothesis, d40_30mer was passivated by coating it with polylysine-PEG polymer, which has been used in vivo with other DNA origami formulations for stabilization, charge neutralization, and avoidance of macrophage uptake.

PEGylated d40_30mer exhibited enhanced stability in mouse serum and reduced association with murine macrophage cells in vitro. However, d40_30mer nanoparticles with or without PEGylation showed similar patterns of early accumulation in medullary and subcapsular sinus regions, and PEG-coated DNA-VLPs elicited weaker germinal centers compared to the uncoated d40_30mer, likely due to interference of the polylysine-PEG coating with antigen recognition on BCRs.

Heavily glycosylated nanoparticle-antigens become decorated with complement in vivo via the lectin pathway, when mannose binding lectin (MBL, an innate immune protein present in blood and lymph) binds to the particles and triggers complement deposition. To test the hypothesis of insufficient complement activation by the DNA-VLPs, binding of MBL and C3 from naive mouse serum to eOD-GT8 monomer, d40_30mer, or p60mer particles immobilized on ELISA plates was measured. MBL and C3 both showed significantly lower binding to eOD-GT8 monomer and d40_30mer compared to p60mer (. Similarly, very little binding of recombinant MBL to eOD-GT8 monomer or d40_30mer was detected by ELISA compared to p60mer. Thus, DNA-VLPs exhibited considerably less effective engagement of the lectin pathway for complement activation than the p60mer nanoparticles.

Engineering DNA-VLPs with High Antigen Density Induces Follicle Targeting and Augments Antigen-Specific GC B Cell Responses

It was hypothesized that poor complement activation explained the lack of FDC accumulation of the DNA-VLPs, and that this was a result of insufficient antigen density on the DNA origami scaffold.

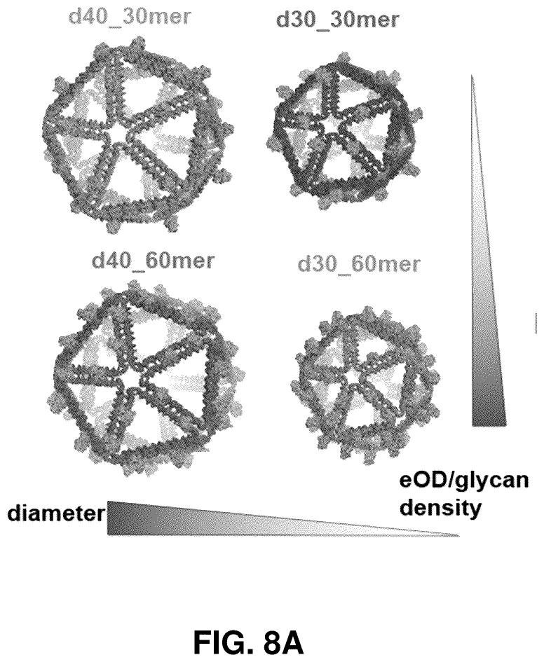

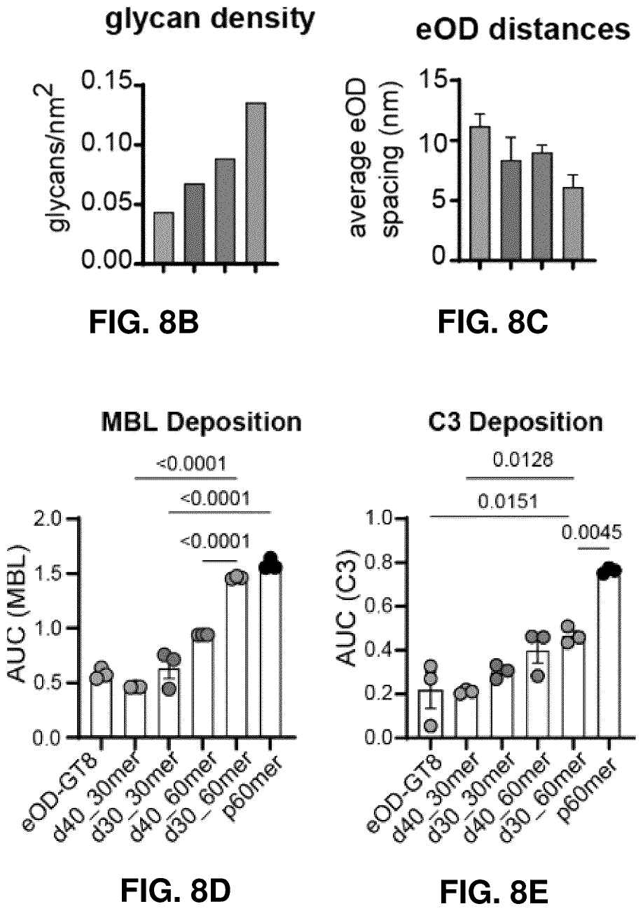

MBL is a large protein complex consisting of trimeric stalks containing a collagen domain and carbohydrate-binding domain (CBD), which oligomerize forming hexamers. Each CBD in the stalk trimer is estimated to be −5 nm apart from its neighbor with similar or larger distances to CBDs in neighboring stalks of the oligomer. Each CBD has weak, millimolar affinity for mannose, but strong binding can be achieved through the avidity of multiple CBD stalks binding to numerous glycans; thus, MBL binding is highly sensitive to the density of glycans on a viral or bacterial surface. To assess the role of antigen density on MBL binding and complement activation by DNA-VLPs, a set of 4 different particles was synthesized, with diameters of approximately -23 or −34 nm, functionalized with either 30 or 60 copies of eOD-GT8 (FIG. 8A), providing a range of predicted glycan densities (0.04 to 0.14 glycans/nm²) and a range of inter-antigen distances from 11 nm (the original d40_30mer) down to 4.5-6 nm between neighboring antigens (d30_60mer, FIGS. 8B, 8C).

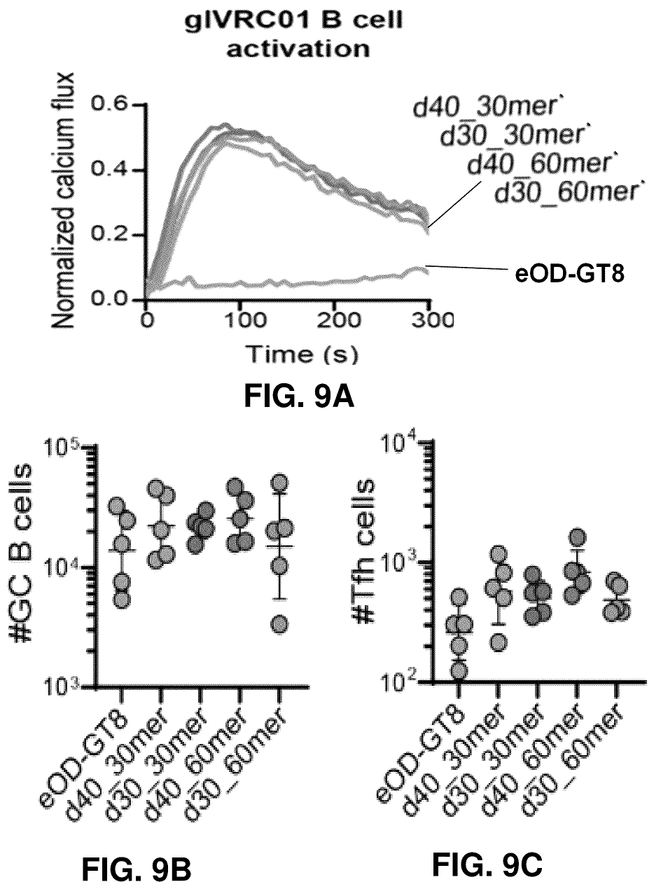

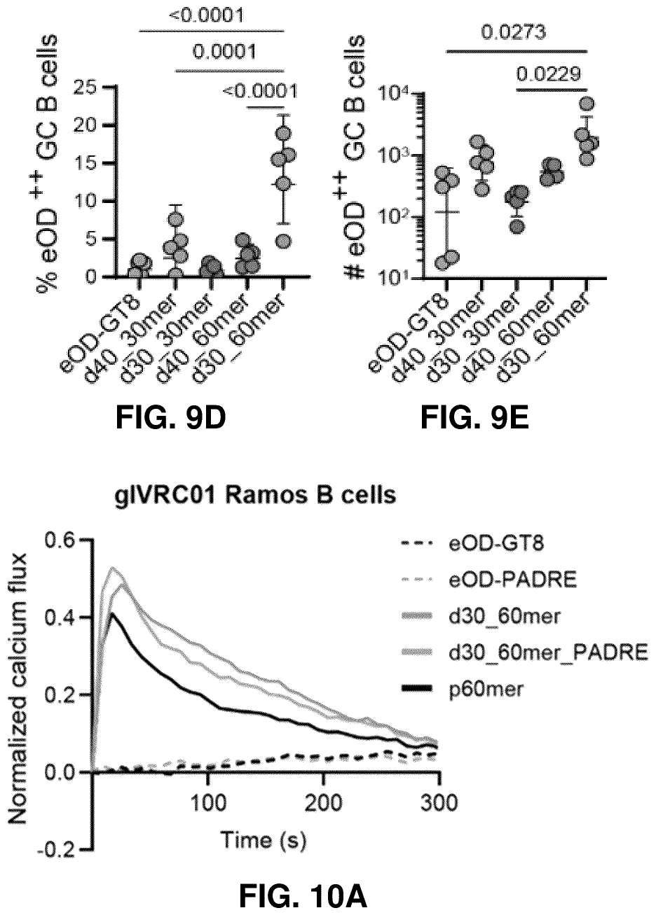

Upon antigen conjugation, which was quantified by BCA, the hydrodynamic diameter of these particles was measured by DLS to be approximately −30 nm and −40 nm, respectively, denoted as d30 and d40. Each of the 4 DNA-VLP designs had sufficient avidity to robustly activate cognate B cells and elicited indistinguishable calcium flux in germline VRCO1-Ramos B cells in vitro (FIG. 9A). Amongst these designs, antigen attachment sites in d30_60mer are spaced similarly to the distances between CBD domains on MBL, and this design also closely resembles the geometric configuration of eOD-GT8 antigen on lumazine synthase p60mer.

Complement deposition assays were conducted with fresh mouse serum to evaluate how antigen spacing affected MBL and C3 deposition on the DNA-VLP surfaces, using the p60mer nanoparticle as a positive control for the assay. A monotonic increase in both MBL and C3 deposition on DNA-VLPs was observed as antigen density increased, with the highest amount of complement depositing on d30_60mer (FIGS. 8D, 8E).

MBL binding with d30_60mer was nearly identical to p60mer; however, the amount of C3 opsonized on DNA-VLPs was significantly lower. Next, we immunized mice with the panel of DNA-VLPs and harvested dLNs for cleared lymph node imaging on day 4 after injection. Strikingly, fluorescent eOD-GT8 signal was detected in lymph nodes from all groups, but colocalization with FDCs was only observed for d30_60mer particles. Thus, as previously observed with other synthetically mannosylated protein nanoparticles, stronger deposition of MBL on DNA-VLPs, which can be increased by increasing antigen density, correlates with increased antigen capture on FDCs in vivo.

Next, how antigen density impacts germinal center formation was investigated. Mice immunized with equimolar antigen doses of eOD-GT8 monomer or DNA-VLPs showed similar total counts of GC B cells and Tfh cells two weeks post immunization (FIGS. 9B and 9C). However, a -15-fold increase in the frequency and 10-fold increase in total counts of 20 eOD-specific GC B cells elicited by the d30_60mer was observed compared to the eOD-GT8 monomer control (FIGS. 9D-9E). Thus, a DNA-VLP design that promoted particle capture by FDCs correlated with substantially stronger expansion of antigen-specific B cells in active GCs. Furthermore, this effect was driven by antigen density, rather than valency, since the d40_60mer failed to elicit antigen capture on FDCs or expansion of antigen-specific cells.

### Synthetic T Cell Epitopes Provide Focused T Cell Help to Antigen-Specific GC B Cells

In addition to retention of antigen on FDCs, stimulation of GC B cells by helper CD4 T cells is a critical cue for driving strong germinal center reactions. DNA-VLPs are T-independent scaffolds; therefore, all T cell epitopes must be derived from the conjugated protein antigen. In contrast, protein scaffolds such as lumazine synthase that forms the basis of the p60mer provide abundant scaffold-derived T cell help. Consequently, following p60mer immunization, antigen-specific B cells compete with scaffold-specific B cells for co-stimulation from the same pool of CD4 T cells recognizing scaffold-derived peptides.

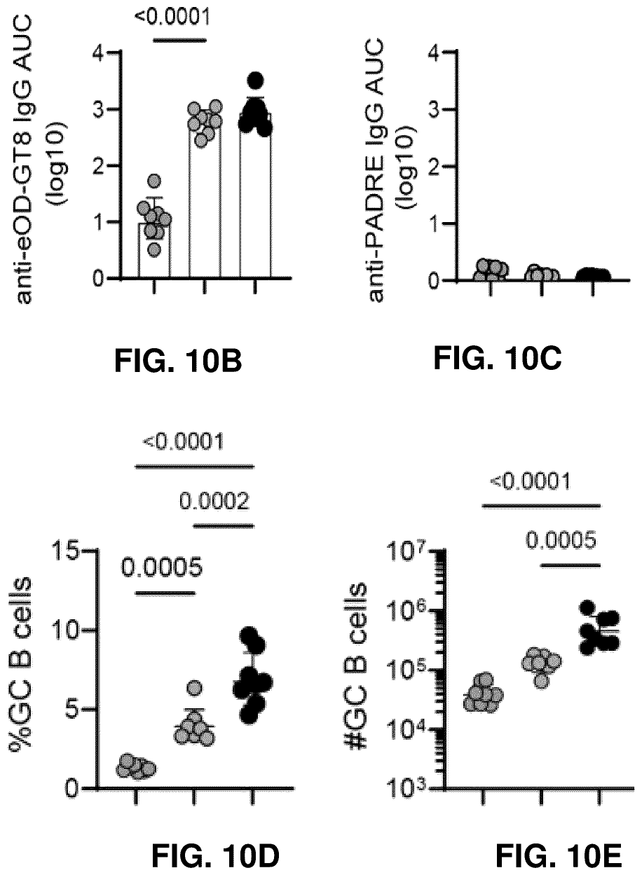

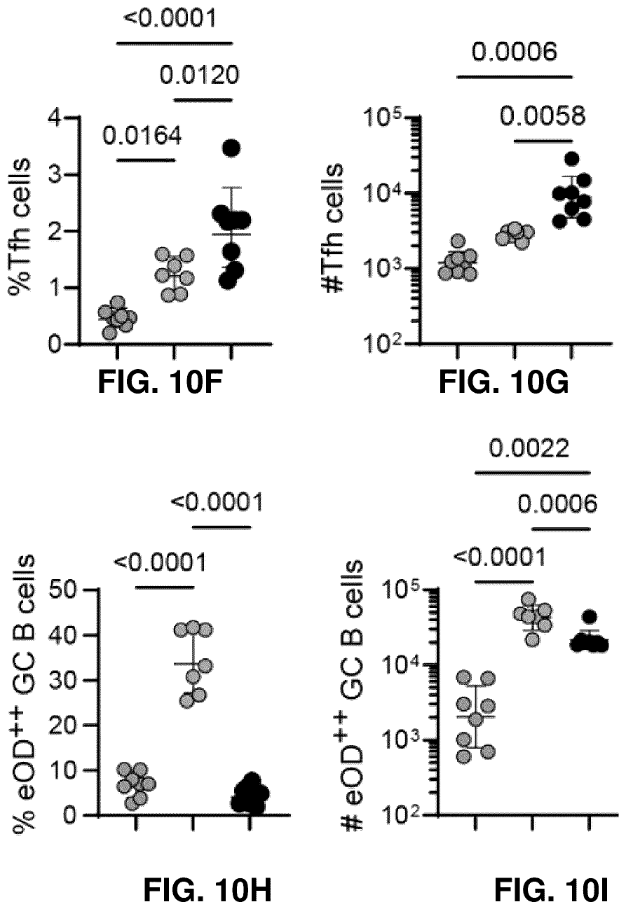

Given the small size of the eOD-GT8 antigen, which may provide insufficient T cell help on its own, whether the incorporation of the universal helper peptide pan-HLA DR-binding epitope (PADRE) on the C-terminus of the antigen could further amplify GC responses elicited by DNA-VLPs without introducing off-target B cell epitopes was next tested. PADRE binds with high affinity to a broad spectrum of human and mouse MHC haplotypes and has been shown to be safe in human clinical trials. This hypothesis was tested by comparing GC responses induced by three different 60mer formulations: d30_60mer, d30_60mer carrying eOD-PADRE (d30_60mer-PADRE), and p60mer. Fusion of the T cell epitope to eOD-GT8 had no effect on formation of the DNA-VLPs (polydispersity, diameter, or antigen functionalization efficiency,). Furthermore, all three 60mers strongly activated germline VRC01 Ramos B cells in vitro (FIG. 10A) and bound MBL and C3 in mouse serum.

Mice were primed with the three 60mer formulations and serum IgG titers assessed by ELISA and GC responses by flow cytometry. The addition of PADRE significantly enhanced early anti-eOD-GT8 IgG responses compared to d30_60mer (FIG. 10B), while minimal IgG responses were detected against the PADRE epitope itself (FIG. 10C). Mice primed with d30_60mer-PADRE also had significantly increased frequencies and total counts of GC B cells and Tfh cells compared to d30_60mer, though the total GC B cell count remained 4-fold lower than that elicited by LumSyn-scaffolded p60mer (FIGS. 10D-10F). However, GCs initiated by d30_60mer-PADRE expanded a much higher frequency of eOD-specific GC B cells (30-40%) compared to p60mer (˜5%, FIG. 10H-10I). This was also reflected in the total counts of eOD-specific GC B cells, which were highest in d30_60mer-PADRE immunizations (2.1-fold greater than p60mer), despite the induction of an overall smaller GC response than that produced by p60mer. Thus, engineering of T cell help in DNA-VLPs enabled priming of a robust antigen-focused GC response.

### DNA-VLPs Prime Epitope-Focused Germinal Centers in Humanized Mice

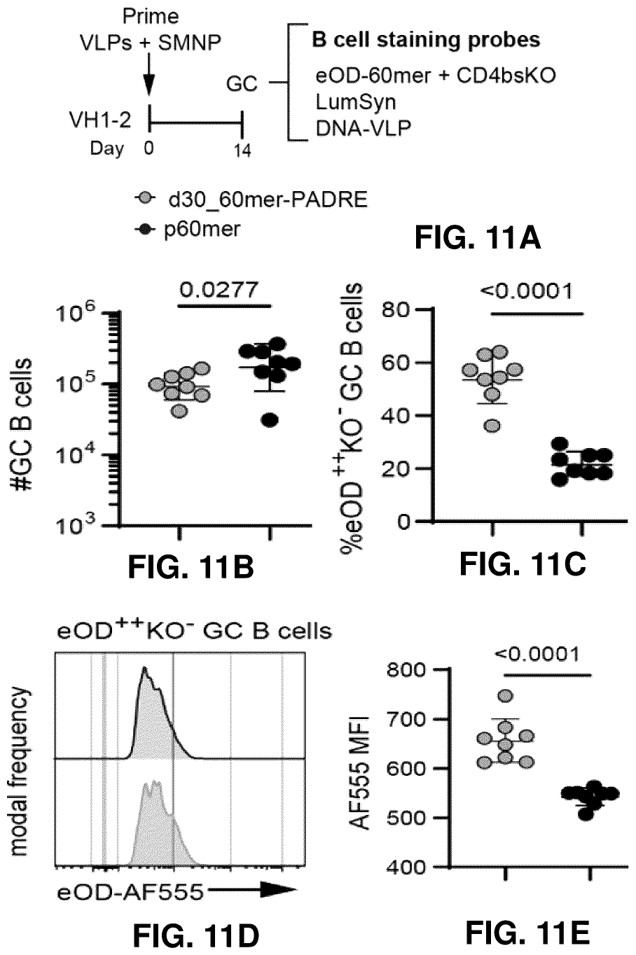

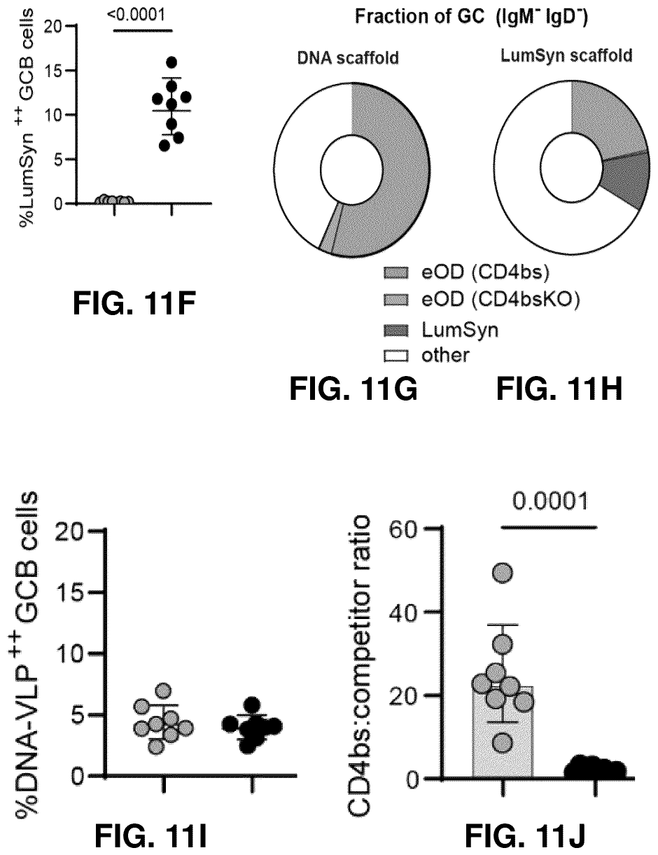

It was hypothesized that the increased frequency of antigen-specific B cells in germinal centers activated by DNA-scaffolded compared to protein-scaffolded antigen may reflect an “immune focusing” effect of DNA-VLP immunization due to the inert nature of the DNA origami scaffold. To directly assess competitor versus “on-target” epitope-specific B cell responses directed to the CD4 binding site (CD4bs), germinal center responses to protein- or DNA-scaffolded immunogens were analyzed in transgenic mice expressing the germline human IGHV1-2*02 gene segment knocked into the mouse Ig locus (VH1-2 mice), which is paired with endogenous mouse light chains. This mouse model is designed to assess the engagement of engineered germline targeting immunogens with a diverse repertoire of VRCO1-class B cell precursors expressing human heavy chains, modeling the diversity of potential bnAb precursor B cells present in the human B cell repertoire, with a low frequency of bona fide bnAb precursors. VH1-2 mice were immunized with d30_60mer-PADRE or p60mer and analyzed their GC responses in draining lymph nodes, using flow probes to characterize antigen-specific vs. competitor B cells in GCs (FIG. 11A).

To assess on-target responses, we analyzed the staining of IgM- IgD- GC B cells by fluorescently labeled eOD-60mer nanoparticle probes and an eOD-60mer-CD4bsKO nanoparticle probe, the latter of which contains mutations in the CD4bs that ablate binding by true VRCO1-class precursor B cells; binding to this KO probe identifies B cells specific to eOD epitopes other than the CD4bs. Importantly, it was previously observed that antibodies isolated by intact eOD-60mer nanoparticle probes were not reactive to bare lumazine synthase. This suggests that scaffold-specific responses primed by p60mer represent B cells which recognize breakdown products of the protein nanoparticle that are rapidly generated by extrafollicular proteases in the lymph node. For quantifying scaffold-specific competitor B cells, fluorescently labeled bare lumazine synthase or bare DNA-VLPs we reused to stain GC B cells. Comparing GC B cell binding to these different sets of nanoparticle probes quantifies relative frequencies of “on-target” CD4bs-specific (eOD++KO-) and “off-target” (CD4bs-KO+ or scaffold-specific) clones in GCs.

As observed in WT mice, the p60mer primed overall slightly larger total GC responses (2-fold greater than the DNA-VLP, FIG. 11B). However, we observed that the composition of GCs elicited by protein- and DNA-scaffolded antigen was very distinct: GCs formed in mice immunized with d30_60mer-PADRE were composed of nearly -60% on-target CD4bs-specific GC B cells (identified by eOD++KO- gate), compared to -20% expanded by p60mer (FIG. 11C). Furthermore, the on-target B cells expanded by the DNA-VLP formulation had stronger staining by the antigen probes represented by higher mean fluorescence intensity, suggesting that these cells might have undergone higher extents of affinity maturation (FIG. 11D). Following p60mer immunization, LumSyn-specific B cells contributed to -13% of the GC, comparable in magnitude to the on-target CD4bs-directed response (FIG. 11E). When staining with bare DNA-VLP probes, some background staining was detected in GC B cells, but this staining was the same in mice primed with either DNA-VLPs and p60mer, and similarly low levels of background binding were detected on B cells from naive mice (FIGS. 11G, 11H), which may reflect low-level scavenger receptor expression in B cells. Compared to the p60mer, DNA-VLP immunization promoted germinal centers that contained 25-fold higher ratios of on-target CD4bs-specific B cells to off-target competitors (FIGS. 11I-11J), whereas in p60mer the frequency of epitope-specific B cells was enriched 2-fold.

### CONCLUSION

A set of DNA-VLP design rules were identified, principally optimizing antigen density, particle size, and incorporating synthetic T cell helper epitopes to trigger follicular targeting of the VLPs, which led to robust, 15-fold greater expansion of antigen-specific GC B cells compared to eOD-GT8 monomer. In a humanized mouse model of VRCOlclass B cell priming, GCs formed by scaffold-silent DNA-VLPs had over 12-fold higher ratios of epitope-specific to competitor B cells compared to the protein-scaffolded eODGT8 60mer. DNA-VLPs effectively expanded VRCO1-class precursors within 14 days following low-dose immunization, conditions where the protein nanoparticle completely failed to expand bnAb-lineage B cells. Thus, eliminating competitive scaffold-specific humoral responses can have a substantial impact on the priming of rare B cell precursors, with DNA-VLPs showing promise as a platform technology to prime antigen-focused germinal center responses.

Anti-scaffold B cell responses have been previously reported for almost all protein-based nanoparticle scaffolds, and their effects on limiting on-target immune responses and, consequently, the development of a successful vaccine against pathogens such as HIV, are still incompletely understood. Thus far, anti-scaffold antibodies have been thought to be minimally concerning for immunodominant antigens, or potentially even beneficial by improving antigen capture in lymph nodes or masking scaffold epitopes in future boosts. However, for subdominant antigens like HIV Env, anti-scaffold responses have been shown to dominate the serum antibody response, and it has not yet been investigated how these competitor B cells influence primary GC dynamics. While strategies such as glycosylation, PEGylation, and PASylation have been introduced to reduce scaffold-specific responses, such approaches are known to be imperfect. Further, even with perfect masking of exposed scaffold epitopes on assembled nanoparticles, degradation of these nanoparticles by tissue proteases that are abundant in lymph node sinuses may expose new epitopes that contribute to competitor scaffold-specific responses. This phenomenon has been observed with p60mer, which elicits robust anti-LumSyn B cells responses, even though intact particles mostly bind B cells that are not LumSyn-specific. Thus, our understanding of the role of protein scaffold competition on humoral immune responses remains incomplete due to limited tools and materials to test these effects of altering scaffolds without changing properties like geometry or antigen display.

Here, we instead used inert DNA origami vaccine scaffolds to assess the impact of distracting epitopes on the potency of germinal center responses towards a displayed antigen, eOD-GT8. Notably, we found that high valency of 60 antigens and high eOD-GT8 density on DNA-VLPs was critical for engaging complement pathways, promoting follicle retention, and expanding antigen-specific B cells in GCs, whereas larger particles or particles with lower antigen copy numbers failed. Intriguingly, the optimal d30_60mer nanoparticle design closely mimicked the geometry of the clinical p60mer nanoparticle, and both particles were effectively recognized by MBL from mouse serum. However, the amount of C3 on DNA-VLPs was significantly lower compared to the protein nanoparticle, which may arise due to the porosity, surface charge, and interaction of DNA-VLPs with complement binding proteins, which may potentially limit downstream complement activation. This remains a design element that can be further improved for the DNA origami platform. Second, it was found that augmenting the small eOD-GT8 immunogen with synthetic T cell epitopes substantially improved the immunogenicity of DNA-VLPs. Lumazine synthase and other protein scaffolds have been shown to have intrinsic T cell epitopes; it was found that adding a synthetic T cell epitope (PADRE) to the DNA-VLP, could recapitulate scaffold-derived T cell help without introducing off-target epitopes for B cells.

Since the optimized DNA-VLP 60mer matched the clinical p60mer nanoparticles in regard to antigen valency, spacing, nanoparticle geometry, and presence of T cell help, it was also possible to assess the effect of the scaffold material on the subsequent clonal competition in GC responses. GCs expanded by DNA-VLPs were significantly more epitope-specific than those induced by the LumSyn-based protein nanoparticle. T focusing effect was attribute due to the undiluted T cell help provided to the eOD-specific B cells, whereas in p60mer vaccination, antigen- and scaffold-specific B cells compete for LumSyn-derived Tfh help. The ratio of on-target CD4bs-specific B cells to off-target competitors (scaffold-specific B cells or non-CD4bs specific B cells) was characterized in the VH1-2 rearranging mouse model by staining lymphocytes with antigen and scaffold-only flow cytometry probes. GCs primed by DNA-VLPs were significantly more focused on the displayed antigen compared to GCs generated by p60mer, where lumazine-specific B cells competed in the GC at high frequency. The BCR sequencing analysis confirms that epitope-focusing of the GCs enhances the priming of bnAb precursors, as evidenced by the increased number of VRCO1-class precursors expanded by immune focused GCs primed with DNA-VLPs compared to the more competitive GCs primed by p60mer. Furthermore, DNA-VLPs reproducibly primed these VRCO1 precursors across multiple animals.

Collectively, the results emphasize the importance of nanoscale antigen organization on lymph node delivery and illustrate that scaffold-specific competitor B cells may significantly influence clonal competition dynamics in GCs and the ability to prime desirable subdominant B cell clones, with currently unknown yet important potential consequences on limiting successful vaccine design for challenging pathogens such as HIV.

### Example 9: Multimerization of Core-g28v2 on DNA-VLPs

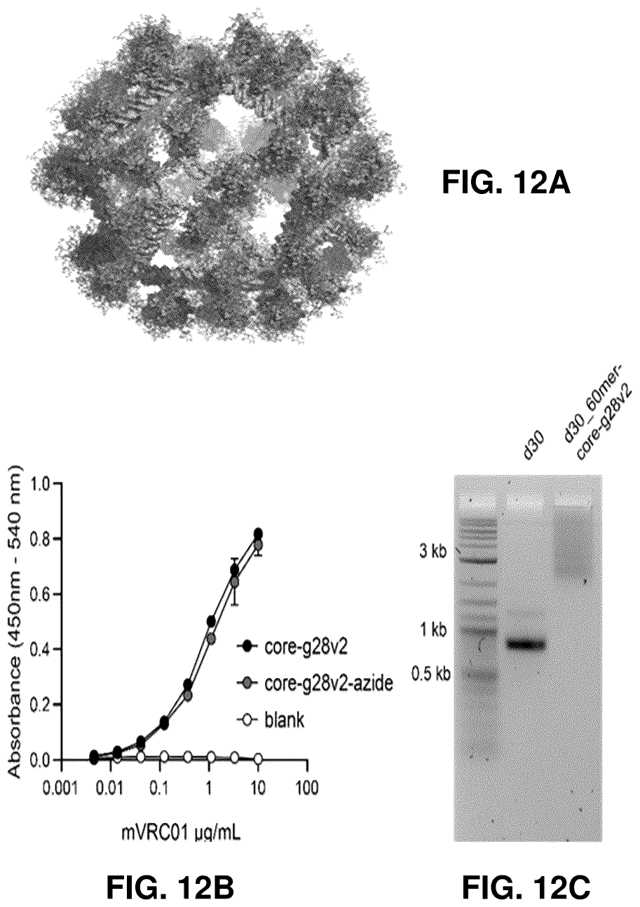

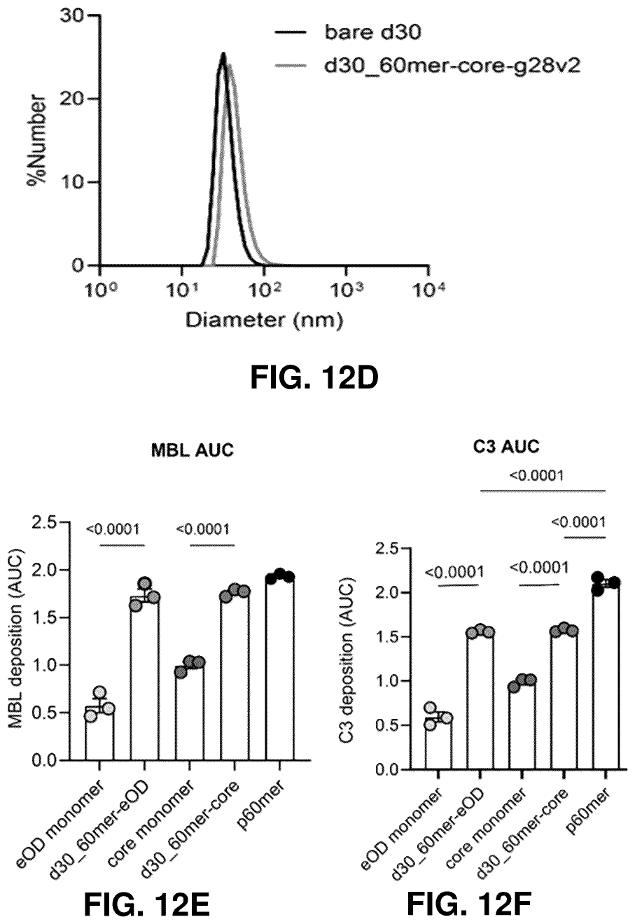

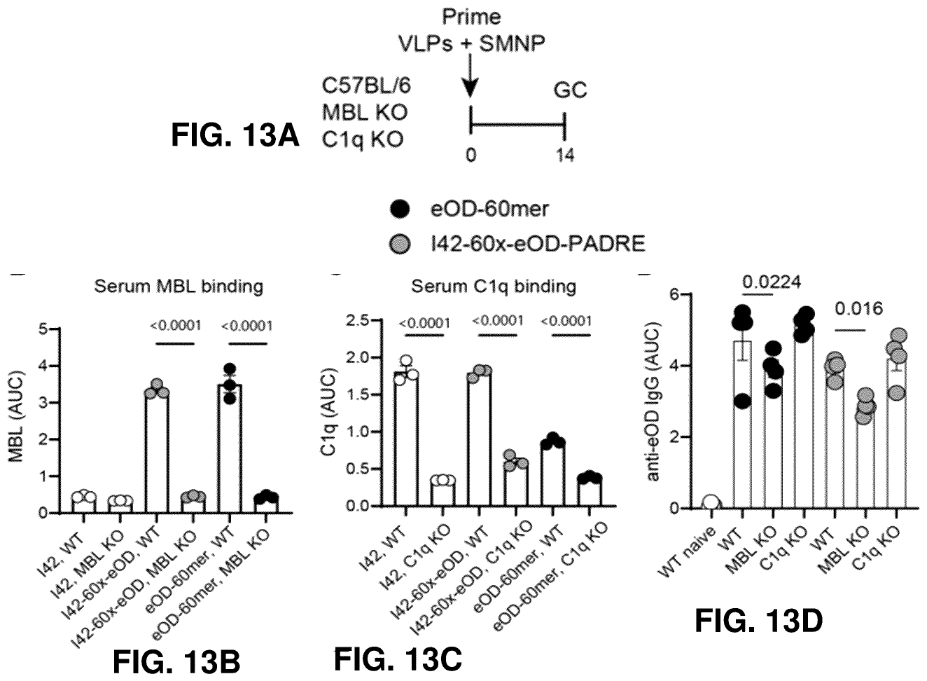

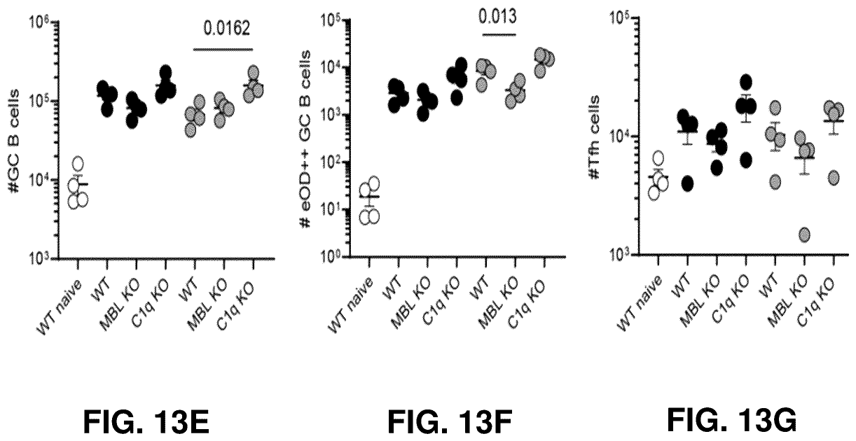

To further demonstrate the versatility and translational potential of DNA-VLPs as modular vaccine scaffolds, the engineered HIV immunogen core-g28v2 was multimerized onto icosahedral d30 nanoparticles using established SPAAC chemistry. This generated 5 d30_60mer-core-g28v2, comprising 60 copies of core-g28v2 displayed on each VLP (FIG. 12A). Core-g28v2 is a rationally designed, Env-derived gpl20 core immunogen developed to serve as a boosting antigen for VRCO1-class B cells initially primed with eOD-based immunogens. It has been evaluated in both rigorous preclinical mouse models and in the G002 human clinical trial.

It was first confirmed that installation of an C-terminal azide via reduction and azido-PEG-maleimide linker modification did not disrupt recognition of the CD4 binding site by murine VRCO1 antibodies, indicating preserved immunogenicity (FIG. 12B). The azide-modified core-g28v2 was then conjugated to DBCO-functionalized d30 scaffolds, and excess protein was removed by drop dialysis, following protocols established for eOD-GT8.

Successful multimerization was validated by agarose gel electrophoresis (FIG. 12C), dynamic light scattering (FIG. 12D), and BCA protein quantification. The resulting protein-decorated DNA-VLPs were visualized by negative stain transmission electron microscopy. Whether the antigen density on d30_60mer-core-g28v2 was sufficient to activate the lectin complement pathway that promotes follicle targeting was also assessed, as previously demonstrated with eOD-GT8. Using a complement deposition ELISA using freshly isolated mouse serum, a marked increase in both mannose-binding lectin (MBL) (FIG. 12E) and complement component C3 deposition (FIG. 12F) was observed on d30_60mer-core-g28v2, relative to its monomeric counterpart. These results suggest that the high-density glycan presentation on DNA-VLPs enables lectin pathway activation across diverse HIV immunogens, not limited to the specific glycosylation features of eOD-GT8.

### REFERENCES

1 Burton, Nat. Rev. Immunol. 19, 77-78, doi:10.1038/s41577-018-0103-6 (2019).
2 McGuire, et al., J Virol. 89 (2015).
3 Nguni, et al., Major Scientific Hurdles in HIV Vaccine Development: Historical Perspective and Future Directions. Front. Immunol. 11, 590780, doi:10.3389/fimmu.2020.590780 (2020).
4 Simek, et al., J Virol. 83, 7337-7348 (2009).
5 Haynes, et al. Nature Reviews Immunology 23, 142-158, doi:10.1038/s41577-022-00753-w (2023).
6 Nieuwenhuis & Opstelten, Am. J. Anat. 170, 421-435, doi:10.1002/aja.1001700315 (1984).
7 Victora & Nussenzweig, Annu. Rev. Immunol. 40, 413-442, doi:10.1146/annurev-immunol-120419-022408 (2022).
8 Jardine, et al. Science 340, 711-716, doi:10.1126/science.1234150 (2013).
9 Jardine, et al. HIV-1 Science 351, 1458-1463, doi:10.1126/science.aad9195 (2016).
10 Duan, et al. Immunity 49, 301-311. (2018).
11 Irvine & Read, Current Opinion in Immunology 65, I-6, doi:https://doi.org/10.1016/j.coi.2020.01.007 (2020).
12 Kato et al. Immunity 53, 548-563.e548, doi:10.1016/j.immuni.2020.08.001 (2020).
13 Bennett, et al., ACS Chem. Biol. 10, 1817-1824, doi:10.1021/acschembio.5b00239 (2015).
14 Bachmann & Jennings, Nat. Rev. Immunol. 10, 787-796, doi:10.1038/nri2868 (2010).
15 Brooks et al. Nature Immunology 24, 1762-1777, doi:10.1038/s41590-023-01597-9 (2023).
16 Ellis, et al. Cell Reports 42, 113552, doi:https://doi.org/10.1016/j.celrep.2023.113552 (2023).
17 Veneziano, et al. Nat. Nanotechnol. 15, 716-723, doi:10.1038/s41565-020-0719-0 (2020).
18 Gonzalez, et al. Annu. Rev. Immunol. 29, 215-233, doi:10.1146/annurev-immunol-031210-101255 (2011).
19 Abbott & Crotty, Immunological Reviews 296, 120-131, doi:https://doi.org/10.1111/imr.12861 (2020).
20 Du et al. Innate Immune Stimulation Using 3D Wireframe DNA Origami. ACS Nano (2022).
21 Wamhoff et al. Nature Communications 15, 795, doi:10.1038/s41467-024-44869-0 (2024).
22 Knappe et al., ACS Nano 15, 14316-14322, doi:10.1021/acsnano.lc03158 (2021).
23 Read et al., Cell Reports 38, 110217, doi:https://doi.org/10.1016/j.celrep.2021.110217 (2022).
24 Tokatlian, et al. Science 363, 649-654, doi:10.1126/science.aat9120 (2019).
25 Alexander, et al. The Journal of Immunology 164, 1625-1633, doi:10.4049/jimmunol.164.3.1625 (2000).
26 Qingqing, et al. bioRxiv, 2023.2008.2024.554561, doi:10.1101/2023.08.24.554561 (2023).
27 Oktay, et al. Communications Biology 6, 308, doi:10.1038/s42003-023-04689-2 (2023).
28 Tam, et al. Proc. Natl. Acad. Sci. U.S.A 113, E6639-E6648, doi:10.1073/pnas.1606050113 (2016).
29 Kranich & Krautler, N. J. How Follicular Dendritic Cells Shape the B-Cell Antigenome. Front. Immunol. Front Immunol (2016).
30 Leggat, al. Science 378, eadd6502, doi:10.1126/science.add6502 (2022).
31 Schiffner, et al. bioRxiv, 2024.2001.2022.575410, doi:10.1101/2024.01.22.575410 (2024).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A nucleic acid nano-structured virus like particle (NANVLP) for stimulation of germinal center B cells (GCB), comprising:
(i) a nucleic acid nanostructure (NAN);
(ii) a plurality of antigen molecules,
wherein the plurality of antigen or engineered immunogen molecules are displayed on the NAN surface; and
(iii) optionally one or more helper T cell epitope designed to elicit GCB; and
(iv) optionally densely glycosylated antigens or synthetic oligomannose structures conjugated to NANVLP surface for complement system activation,
wherein the plurality of antigen molecules and optionally the one or more T cell epitope are configured for stimulation of antigen-specific B cells.

2. The NANVLP of claim 1, wherein the plurality of antigen molecules are present on the NAN surface at a density of between about 0.04 and about 0.14 molecules/nm², inclusive.

3. The NANVLP of claim 1, wherein the plurality of antigen molecules are evenly distributed on the NAN surface, and
wherein the distance between two molecules of the plurality of antigen molecules on the NAN surface is between about 10 nm and about 4 nm, inclusive.

4. The NANVLP of claim 1, wherein the NAN comprises a single stranded nucleic acid scaffold sequence and a plurality of single stranded nucleic acid staple strands that hybridize to the scaffold sequence to form the three-dimensional nanostructure having a defined geometric shape.

5. The NANVLP of claim 4, wherein the geometric shape is selected from the group consisting of a helix bundle, cuboidal structure, icosahedral structure, tetrahedral structure, cuboctahedral structure, octahedral structure, and hexahedral structure.

6. The NANVLP of claim 1, wherein, wherein the NAN has a diameter of between about 20 nm and about 100 nm, inclusive; or from about 20 nm to about 30 nm, inclusive.

7. The NANVLP of claim 6, wherein the plurality of antigen comprises from 10 to 200 molecules of antigen, inclusive, or 60 molecules of antigen.

8. The nanostructure of claim 1, wherein the plurality of antigen molecules and/or the one or more T cell epitope are covalently or non-covalently bound to the NAN.

9. The nanostructure of claim 4, wherein the plurality of antigen molecules and/or the one or more T cell epitope are indirectly or directly associated with the NAN via nucleic acid overhangs extending from the 3′ or 5′ ends of one or more selected staple strands of the nanostructure or via covalent conjugation chemistries.

10. The NANVLP of claim 1, wherein an antigen molecule of the plurality of antigen molecules is derived from the group consisting of a small molecule, a polypeptide, a protein, a nucleic acid, a lipid, a carbohydrate and a synthetic polymer,
optionally wherein the small molecule is a hapten.

11. The NANVLP of claim 1, wherein an antigen molecule of the plurality of antigen molecules is derived from the group consisting of a virus, a protozoan, a bacterium, a fungus, and a cancer,
optionally wherein the virus is selected from the group consisting of influenza, dengue viruses, Hepatitis C virus, picornaviruses, coronaviruses and human immunodeficiency virus (HIV).

12. The NANVLP of claim 1, wherein an antigen molecule of the plurality of antigen molecules is HIV immunogens engineered outer domains (eODs), core-g28v2, or SOSIP trimers.

13. The NANVLP of claim 1, wherein synthetic high mannose glycans are positioned at a density of from about 0.04 to about 1 molecules/nm², independently of protein antigen.

14. The NANVLP of claim 1, comprising one or more helper T cell epitope(s), optionally wherein the T cell epitope(s) is conjugated to or complexed with an antigen molecule of the plurality of antigen molecules.

15. The NANVLP of claim 14, wherein each antigen molecule of the plurality of antigen molecules is conjugated to a T cell epitope designed to elicit GCB.

16. The NANVLP of claim 15, wherein the T cell epitope is a pan HLA DR-binding epitope (PADRE) peptide,
optionally wherein the PADRE peptide comprises the amino acid sequence AKFVAAWTLKAAA.

17. A thymus-independent nucleic acid nano-structured virus like particle (NANVLP) for stimulation of germinal center B cells (GCB), comprising:
(i) a nucleic acid nanostructure (NAN) comprising a single stranded nucleic acid scaffold sequence and a plurality of single stranded nucleic acid staple strands that hybridize to the scaffold sequence to form the three-dimensional nanostructure having a defined icosahedral shape of about 23 nm;
(ii) 60 antigen molecules linked to the NAN,
wherein the antigen molecules are displayed on the NAN surface at a density of about 0.14 molecules/nm² and with an inter-antigen distance of between about 4 nm and about 6 nm, inclusive, and
wherein each antigen molecule is conjugated to a PADRE polypeptide or other T cell epitope.

18. The NANVLP of claim 17, wherein the antigen comprises HIV eOD-GT8,
optionally wherein the eOD-GT8 molecules are covalently linked to the NAN,
optionally wherein the covalent linkage is formed by maleimide-thiol coupling, strain-promoted azide-alkyne cycloaddition, or inverse electron-demand diels-alder reactions.

19. A pharmaceutical formulation comprising the NANVLP of claim 1, and a pharmaceutically acceptable excipient for administration in vivo.

20. A vaccine comprising the pharmaceutical formulation of claim 19,
optionally further comprising an adjuvant.

21. A method for generating an immune response in vivo against a sub-dominant epitope comprising administering to a subject the vaccine of claim 17,
wherein the vaccine is administered in an effective amount to increase in a subject one or more of
(i) an antigen-specific antibody response, or
(ii) a response in epitope-specific germinal center B cell frequency, or
(iii) plasmablast frequency, or increase memory B cell frequency, or (iv) somatic hypermutation rates of these B cells, or
(v) inflammatory cytokine expression, as compared to a control, non-NAN-based vaccine against the same antigen.

22. The method of claim 21, wherein the antigen-specific antibody response comprises increasing or stimulating one or more of antigen specific IgG antibodies selected from group consisting of, IgG1, IgG2, IgG3 and IgG4, or a combination thereof.

23. The method of claim 21, wherein the increase of a response in germinal centers comprises increasing frequency and counts of epitope-specific germinal center B cells, increasing frequencies and/or activation T follicular helper (Tfh) cells, increasing B cell proliferation or residence in dark zone of germinal centers, increasing somatic hypermutation, or a combination thereof.

24. The method of claim 21, wherein the expression of inflammatory cytokines comprises an increase or stimulation of expression of one or more cytokine selected from the group consisting of IL-6, IL-21, IFN-γ, IFN-α, IL-1β, TNF-α, and CXCL10 (IP-10).

25. The method of claim 21, wherein the immune response further comprises reducing undesired competitor B cell responses as compared to the control vaccine.


SEQUENCE LISTING (21 sequences)

SEQ ID NO: 1 - DNA, 20 bp, synthetic construct
  source 1..20: mol_type = other DNA
ccctttagggttccgattta

SEQ ID NO: 2 - DNA, 20 bp, synthetic construct
  source 1..20: mol_type = other DNA
tctttgccttgcctgtatga

SEQ ID NO: 3 - DNA, 20 bp, synthetic construct
  source 1..20: mol_type = other DNA
gctgaaaaggtggcatcaat

SEQ ID NO: 4 - DNA, 19 bp, synthetic construct
  source 1..19: mol_type = other DNA
gctaacgagcgtctttcca

SEQ ID NO: 5 - protein, 15 aa, synthetic construct
LRFGIVASRANHALV

SEQ ID NO: 6 - protein, 32 aa, synthetic construct
ILMQYIKANSKFIGIPMGLPQSIALSSLMVAQ

SEQ ID NO: 7 - protein, 15 aa, synthetic construct
RIQRGPGRAFVTIGK

SEQ ID NO: 8 - protein, 13 aa, synthetic construct
PKYVKQNTLKLAT

SEQ ID NO: 9 - protein, 21 aa, synthetic construct
FNNFTVSFWLRVPKVSASHLE

SEQ ID NO: 10 - protein, 13 aa, synthetic construct
TPPAYRPPNAPIL

SEQ ID NO: 11 - protein, 15 aa, synthetic construct
EKKRKRSQRYDPGRV

SEQ ID NO: 12 - protein, 16 aa, synthetic construct
PIFFLHHSNTDRLWAI

SEQ ID NO: 13 - protein, 10 aa, synthetic construct
LYNLRRGTAL

SEQ ID NO: 14 - protein, 13 aa, synthetic construct
SAGVYQILAIYST

SEQ ID NO: 15 - protein, 10 aa, synthetic construct
IVAQSIALSS

SEQ ID NO: 16 - protein, 17 aa, synthetic construct
KKQYIKANSKFIGITEL

SEQ ID NO: 17 - protein, 23 aa, synthetic construct
DSETADNLEKTVAALSILPGHGC

SEQ ID NO: 18 - protein, 25 aa, synthetic construct
ALNIWDRFDVFCTLGATTGYLKGNS

SEQ ID NO: 19 - protein, 13 aa, synthetic construct
AKFVAAWTLKAAA

SEQ ID NO: 20 - protein, 13 aa, synthetic construct
AAATLWKAAKFVA

SEQ ID NO: 21 - protein, 15 aa, synthetic construct
  SITE 1: note = D-alanine
  SITE 2: note = D-lysine
  SITE 3: note = beta-cyclohexyl-L-alanine
  SITE 13: note = D-alanine
  SITE 14: note = Acp
AKXVAAWTLKAAAXC